SEQUENCE LISTING (34 sequences)

SEQ ID NO: 1 - protein, 87 aa, Mus musculus
SAQLLKSVFVKNVGWATQLTSGAVWVQFNDGSQLVVQAGVSSISYTSPDGQTTRYGENEKLPEYIKQKLQCLSSILLMFSNPTPNFQ

SEQ ID NO: 2 - protein, 89 aa, Homo sapiens
SAQLLKSVFVKNVGWATQLTSGAVWVQFNDGSQLVVQASMQVSSISYTSPNGQTTRYGENEKLPDYIKQKLQCLSSILLMFSNPTFNFH

SEQ ID NO: 3 - protein, 87 aa, Drosophila melanogaster
QNIPIKRINVPEIGIATELSHGVVQVQFYDGSVVSVIPGGGGITYTQPNGTSTHFGKGDDLPFPVRDRVGQIPNIQLKLKTAPLLGS

SEQ ID NO: 4 - protein, 86 aa, Danio rerio
SGKVLKSIFVPNIGWASQLTTGEVWVQFNDGSQLMVQVGVSCIIFTSPEGHITRYKENEKLPELVKEKLHCLSSILGLLANPAARC

SEQ ID NO: 5 - protein, 87 aa, Rattus norvegicus
SAQLLKSVFVKNVGWATQLTSGAVWVQFNDGSQLVVQAGVSSISYTSPDGQTTRYGENEKLPEYIKQKLQCLSSILLMFSNPTPSFQ

SEQ ID NO: 6 - protein, 86 aa, Drosophila melanogaster
FWISKWVDYSDKYGFGYQLCDEGIGVMFNDTTKLILLPNQINVHFIDKDGKETYMTTTDYCKSLDKKMKLLSYFKRYMIEHLVKAG

SEQ ID NO: 7 - protein, 86 aa, Mus musculus
FWVSKWVDYSDKYGLGYQLCDNSVGVLFNDSTRLILYNDGDSLQYIERDGTESYLTVSSHPNSLMKKITLLNYFRNYMSEHLLKAG

SEQ ID NO: 8 - protein, 86 aa, Homo sapiens; identical to SEQ ID NO: 7

SEQ ID NO: 9 - protein, 86 aa, Rattus norvegicus
FWVSKWVDYSDKYGLGYQLCDNSVGVLFNDSTRLILYNDGDSLQYIERDGTESYLTVSSHPNSLMKKITLLKYFRNYMSEHLLKAG

SEQ ID NO: 10 - protein, 86 aa, Xenopus
FWISKWVDYSDKYGLGYQLCDNSVGVLFNDSTRLIMYNDGDSLQYIERNNTESYLNVRSYPTTLTKKITLLKYFRNYMSEHLLKAG

SEQ ID NO: 11 - protein, 87 aa, Caenorhabditis elegans
FWISKWVDYSDKYGIGYQLCDNSVGVLFNDNSRIMLDQAGNELTYIEKSNKEHYFSMHSEMPGLLNKKVTLLKYFRSYMNDHLVKAG

SEQ ID NO: 12 - protein, 86 aa, Caenorhabditis elegans
FWVSQWVHFPNHGIGYRLCENSSGMLFNDNTQMKVNSAGNQLTFVDENNTEQFYTMNDQVPMNLQTKINIFSNVQSYMNTHLEADT

SEQ ID NO: 13 - protein, 86 aa, Hemicentrotus pulcherrimus
LWVSKWVDYSDKYGLGYQLCDGSVGVLFNDSTRLLLHANADTLEYIERDGNEKYCRLGSYDSTLHKKVTLLKYFRNYMSEHLLKAG

SEQ ID NO: 14 - protein, 86 aa, Mus musculus
VWVSKWVDYSNKFGFGYQLSSRRVAVLFNDGTHMALSANRKTVHYNPTSTKHFSFSMGSVPRALQPQLGILRYFASYMEQHLMKGG

SEQ ID NO: 15 - protein, 86 aa, Homo sapiens
VWVSKWVDYSNKFGFGYQLSSRRVAVLFNDGTHMALSANRKTVHYNPTSTKHFSFSVGAVPRALQPQLGILRYFASYMEQHLMKGG

SEQ ID NO: 16 - protein, 86 aa, Mus musculus
QWVTKWVDYSNKYGFGYQLSDHTVGVLFNNGAHMSLLPDKKTVHYYAELGQCSVFPATDAPEQFISQVTVLKYFSHYMEENLMDGG

SEQ ID NO: 17 - protein, 86 aa, Homo sapiens
QWVKTWVDYSNKYGFGYQLSDHTVGVLFNNGAHMSLLPDKKTVHYYAELGQCSVFPATDAPEQFISQVTVLKYFSHYMEENLMDGG

SEQ ID NO: 18 - protein, 87 aa, Trypanosoma brucei
VWVTSFADFSEKYGLCYRLSTGHTGVHFNDSTKMVWEPITNRVEYYMRVKEVVARGNANMFPESLTKKVTLIKYFKSYLSRTRNSHT

SEQ ID NO: 19 - protein, 87 aa, Schizosaccharomyces pombe
LFITKWVDYSNKYGLGYQLSDESVGVHFNDDTSLLFSADEEVVEYALHPKDTEIKPYIYKVPESIRSKLQLLKHFKSYMGQNLSKAV

SEQ ID NO: 20 - protein, 87 aa, Saccharomyces cerevisiae
MIVTKWVDYSNKHGFSYQLSTEDIGVLFNNGTTVLRLADAEEFWYISYDDREGWVASHYEKPRELSRHLEVVDFFAKYMKANLSRVS

SEQ ID NO: 21 - protein, 85 aa, Drosophila melanogaster
RMPHLHSWFRTTCAVVMHLTNGSVQLNFSDHMKLILCPRMSAITYMDQEKNFRTYRFSVGVSKDLYQKIRYAQEKLRKMLEKMFT

SEQ ID NO: 22 - protein, 88 aa, Mus musculus
RLPYLRTWFRTRSAIILHLSNGTVQINFFQDHTKLILCPLMAAVTYINEKRDFQTYRLSLGCCKELASRLRYARTMVDKLLSSRSASN

SEQ ID NO: 23 - protein, 88 aa, Homo sapiens
RLPYLRTWFRTRSAIILHLSNGSVQINFFQDHTKLILCPLMAAVTYIDEKRDFRTYRLSLGCCKELASRLRYARTMVDKLLSSRSASN

SEQ ID NO: 24 - protein, 88 aa, Xenopus
RLPFLRTWFRTRSAIILHLSNGTVQINFFQDHTKIILCPLMAAVSYIDEKREFRTYKLSLGCCKELASRLRYARTMVEKLQSSKSAVA

SEQ ID NO: 25 - protein, 88 aa, Caenorhabditis elegans
RLPTLRVWFRTKSAIVLHLSNGTVQINFFNDHVKMMMCPLMQAVTFIDQNKRMLTYKLNNGCPEKFLHRLKYAKTMIERLMSDANVVS

SEQ ID NO: 26 - protein, 88 aa, Caenorhabditis elegans
RLPTIQNWFETKSAVIFHLSNGTVQINFLDHVKMVLCPLLKSVTRIEENKRVSTFTFANSCPIKKYLSRIEYAQAKIKLLRPTNNQEH

SEQ ID NO: 27 - protein, 89 aa, Hemicentrotus pulcherrimus
RLPFLQSWFRTKSAIVLHLSNGTVQINFFFEDHTKLIVCPMMGAATYIDAKRNFRTFRLNLGCTPDLYDRIKYANNMVKNMLDKKTTTA

SEQ ID NO: 28 - protein, 84 aa, Mus musculus
APPLLLQWVKTDQALLMLFSDGTVQVNFYGDHTKLILSGWEPLLVTFVARNRSACTYLASHGCSPDLRQRLRYALRLLRDQSPA

SEQ ID NO: 29 - protein, 84 aa, Homo sapiens; identical to SEQ ID NO: 28

SEQ ID NO: 30 - protein, 84 aa, Mus musculus
PRLYLLQWLKSDKALMMLFNDGTFQVNFYHDHTKIIICNQSEEYLLTYINEDRISTTFRLTTGCSSELKNRMEYALNMLLQRCN

SEQ ID NO: 31 - protein, 84 aa, Homo sapiens
PRLYLLQWLKSDKALMMLFNDGTFQVNFYHDHTKIIICSQNEEYLLTYINEDRISTTFRLTTGCSSELKNRMEYALNMLLQRCN

SEQ ID NO: 32 - protein, 85 aa, Trypanosoma brucei
DIVYVKRWLITPQAIIFRLSNKTIQVCFHDKAEVILSSESRVVTYTEPTGNRVTMSLSSTRSREIAARLRYTKDILSELIQNRDI

SEQ ID NO: 33 - protein, 86 aa, Schizosaccharomyces pombe
TMLFMQHYLRTRQAIMFRLSNGIFQFNFLDHRKVVISSTARKIIVLDKERERVELPLQEAFSEDLRSRLKYIRETLESWASKMEVS

SEQ ID NO: 34 - protein, 86 aa, Saccharomyces cerevisiae
DDVFLRRYTRYKPFVMFELSDGTFQFNFKDHHKMAISDGGKLVTYISPSHESTTYPLVEYPESNFREKLTLIKEGLKQKSTIVTVD

(19) United States
(12) Patent Application Publication
(10) Pub. No.: US 2005/0085626 A1
(43) Pub. Date: Apr. 21, 2005

(54) Polo domain structure

(75) Inventors: Genie Chung Chi Leung, Newmarket (CA); John William Hudson, Newmarket (CA); Anna Kozarova, Toronto (CA); Frank Sicheri, Toronto (CA); Jim Dennis, Etobicoke (CA)

Correspondence Address: MERCHANT & GOULD PC, P.O. BOX 2903, MINNEAPOLIS, MN, 55402-0903, US

(73) Assignee: Mount Sinai Hospital, Toronto (CA)

(21) Appl. No.: 10/368,133

(22) Filed: Feb. 14, 2003

Related U.S. Application Data
(60) Provisional application No. 60/357,475, filed on Feb. 15, 2002.
(60) Provisional application No. 60/360,704, filed on Feb. 28, 2002.

(51) Int. Cl. G06F 19/00; G01N 33/48; G01N 33/50; C07K 14/705

(52) U.S. Cl. 530/350; 702/19

(57) ABSTRACT

The present invention relates to binding pockets of a polo domain. In particular, the invention relates to a crystal comprising a binding pocket of a polo domain. The crystal may be useful for modeling and/or synthesizing mimetics of a binding pocket or ligands that associate with the binding pocket. Such mimetics or ligands may be capable of acting as modulators of polo family kinases, and they may be useful for treating, inhibiting, or preventing diseases modulated by such kinases.

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to two-, three- or four-dimensional structures of a polo domain. In particular, the invention relates to a crystal comprising a polo domain. The crystal may be useful for modeling and/or synthesizing mimetics of a polo domain or ligands that associate with the polo domain. Such mimetics or ligands may be capable of acting as modulators of activity of polo family kinases, and they may be useful for treating, inhibiting, or preventing diseases modulated by such kinases.

### BACKGROUND

The Polo-like kinases (Plks) include S. cerevisiae Cdc5, S. pombe Plol, Drosophila Polo, and the four mammalian genes Plk1, Prk/Fnk, Snk and Sak. The Plks play multiple and overlapping roles in cell cycle progression [reviewed in refs. 1-3]. Mutation of polo in Drosophila, plol in S. pombe, and cdc5 in S. cerevisiae, cause mitotic defects including monopolar spindles, aberrant chromosome segregation, and failure of cytokinesis [4-8]. The targeted disruption of Sak in mouse is embryonic lethal at gastrulation with cells arresting in late stage mitosis and displaying failure of cytokinesis [9]. In S. cerevisiae, mitotic defects arising from the loss of cdc5 function can be rescued by the heterologous expression of mammalian Plk [10] or Prk/Fnk [11].

The Plks localize to characteristic mitotic structures during cell cycle progression, presumably to promote the interaction of the enzymes with specific substrates and effectors. Plk, Prk/Fnk, Cdc5, Plo1, Polo and Sak localize to centrosomes in early M phase and/or to the cleavage furrow or mother bud neck during cytokinesis [9, 12-17]. Mutational analyses of Cdc5 and Plk1 have demonstrated a requirement and sufficiency of the polo box motifs for sub-cellular localization [13-15]. In addition, these studies have demonstrated a requirement of proper sub-cellular localization for Plk family function. Interestingly, while most Plks possess two polo box motifs, the Sak orthologues possess only one. Since the sub-cellular localization of Sak conforms to that of the other Plks, the functional relevance of this difference remains to be determined.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

### SUMMARY OF THE INVENTION

Applicants have solved the x-ray crystal structure of a polo domain. Solving the crystal structure has enabled the determination of structural features of the polo domain that permit the design of modulators of proteins comprising a polo domain. The crystal structure also enables the determination of structural features in molecules or ligands that interact or associate with the polo domain.

Knowledge of the conformation of the polo domain and binding pockets thereof is of significant utility in drug discovery. The association of natural substrates and effectors with a polo domain and binding pockets thereof may be the basis of many biological mechanisms. The associations may occur with all or any parts of a polo domain. An understanding of the association of a drug with the polo domain or part thereof will lead to the design and optimization of drugs having favorable associations with target polo family kinases and thus provide improved biological effects. Therefore, information about the shape and structure of the polo domain is valuable in designing potential modulators of proteins comprising a polo domain for use in treating diseases and conditions associated with or modulated by the proteins.

The present invention relates to a two-, three- or four dimensional structure of a polo domain, or a binding pocket thereof.

The invention also relates to a crystal comprising a polo domain or binding pocket thereof.

The present invention also contemplates molecules or molecular complexes that comprise all or parts of either one or more a polo domain, or homologs thereof, that have similar structure and shape.

The present invention also provides a crystal comprising a polo domain or binding pocket thereof and at least one ligand. A ligand may be complexed or associated with a polo domain or binding pocket thereof. Ligands include a substrate or analogue thereof or effector. A ligand may be a modulator of the activity of a polo family kinase

In an aspect the invention contemplates a crystal comprising a polo domain or binding pocket thereof complexed with a ligand (e.g. substrate or analogue thereof) from which it is possible to derive structural data for the ligand (e.g. substrate or analogue thereof).

The shape and structure of a polo domain or binding pocket thereof may be defined by selected atomic contacts in the domain or pocket. In an embodiment, the polo domain binding pocket is defined by one or more atomic interactions or enzyme atomic contacts.

An isolated polypeptide comprising a polo domain or binding pocket thereof with the shape and structure of a polo domain or binding pocket thereof described herein is also within the scope of the invention.

The invention also provides a method for preparing a crystal of the invention, preferably a crystal of a polo domain or binding pocket thereof, or a complex of such a domain or binding pocket thereof, and a ligand.

Crystal structures of the invention enable a model to be produced for a polo domain or binding pocket thereof, or complexes or parts thereof. The models will provide structural information about a polo domain, or a ligand and its interactions with a polo domain or binding pocket thereof. Models may also be produced for ligands. A model and/or the crystal structure of the present invention may be stored on a computer-readable medium.

A crystal and/or model of the invention may be used in a method of determining the secondary and/or tertiary structures of a polypeptide or binding pocket thereof with incompletely characterised structure. Thus, a method is provided for determining at least a portion of the secondary and/or tertiary structure of molecules or molecular complexes that contain at least some structurally similar features to a polo domain or binding pocket thereof of the invention. This is achieved by using at least some of the structural coordinates set out in Table 2.

A crystal of the invention may be useful for designing, modeling, identifying, evaluating, and/or synthesizing mimetics of a polo domain or binding pocket thereof, or ligands that associate with a binding pocket. Such mimetics or ligands may be capable of acting as modulators of polo kinase activity, and they may be useful for treating, inhibiting, or preventing conditions or diseases modulated by such kinases.

Thus the present invention contemplates a method of identifying a potential modulator of a polo family kinase comprising the step of applying the structural coordinates of a polo domain or binding pocket thereof, or atomic interactions, or atomic contacts thereof, to computationally evaluate a test compound for its ability to associate with the polo domain or binding pocket thereof, wherein a test compound that is found to associate with the polo domain or binding pocket thereof is a potential modulator. Use of the structural coordinates of a polo domain or binding pocket thereof, or atomic interactions, or atomic contacts thereof to design or identify a modulator is also provided.

The invention further contemplates classes of modulators of polo family kinases based on the shape and structure of a ligand defined in relation to the molecule's spatial association with a polo domain or binding pocket thereof. Generally, a method is provided for designing potential inhibitors of polo family kinases comprising the step of applying the structural coordinates of a ligand defined in relation to its spatial association with a polo domain or binding pocket thereof, to generate a compound that is capable of associating with the polo domain or binding pocket thereof.

It will be appreciated that a modulator of a polo family kinase may be identified by generating an actual secondary or three-dimensional model of a polo domain or binding pocket thereof, synthesizing a compound, and examining the components to find whether the required interaction occurs.

Therefore, the methods of the invention for identifying modulators may comprise one or more of the following additional steps:
(a) testing whether the modulator is a modulator of the activity of polo family kinases, preferably testing the activity of the modulator in cellular assays and animal model assays;
(b) modifying the modulator;
(c) optionally rerunning steps (a) or (b); and
(d) preparing a pharmaceutical composition comprising the modulator.

Steps (a), (b) (c) and (d) may be carried out in any order, at different points in time, and they need not be sequential.

A potential modulator of a polo family kinase identified by a method of the present invention may be confirmed as a modulator by synthesizing the compound, and testing its effect on the polo family kinase in an assay for enzymatic activity. Such assays are known in the art (e.g phosphorylation assays).

A modulator of the invention may be converted using customary methods into pharmaceutical compositions. A modulator may be formulated into a pharmaceutical composition containing a modulator either alone or together with other active substances.

The invention also contemplates a method of treating or preventing a disease or condition associated with polo family kinases in a cellular organism, comprising:
(a) administering a modulator of the invention in an acceptable pharmaceutical preparation; and
(b) activating or inhibiting the polo family kinases to treat or prevent the disease or condition.

The invention provides for the use of a modulator identified by the methods of the invention in the preparation of a medicament to treat or prevent a disease in a cellular organism. Use of modulators of the invention to manufacture a medicament is also provided.

Still another aspect of the present invention provides a method of conducting a drug discovery business comprising:
(a) providing one or more systems for identifying modulators based on the structure of a polo domain or binding pocket thereof;
(b) conducting therapeutic profiling of modulators identified in step (a), or further analogs thereof, for efficacy and toxicity in animals; and
(c) formulating a pharmaceutical preparation including one or more modulators identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:
(a) providing one or more systems for identifying modulators based on the structure of a polo domain or binding pocket thereof;
(b) (optionally) conducting therapeutic profiling of modulators identified in step (a) for efficacy and toxicity in animals; and
(c) licensing, to a third party, the rights for further drug development and/or sales for agents identified in step (a), or analogs thereof.

These and other aspects of the present invention will become evident upon reference to the following detailed description and Tables, and attached drawings.

### DESCRIPTION OF THE DRAWINGS AND TABLES

The present invention will now be described only by way of example, in which reference will be made to the following Figures:

FIG. 1. Structure-based sequence alignment of the Plk family polo domains. The polo domains from Sak orthologs are shown on top, and polo domains one and two from all other Plks are shown in the middle and bottom respectively. The secondary structure of the polo domain of Sak is indicated above the alignment. Residue numbers for the start of each amino acid sequence are shown on the left. Conserved hydrophobic core residues are green or yellow (green denotes hydrophobic residues conserved in all polo domains and yellow denotes hydrophobic residues conserved within the first or second polo domain), Asp residues red, Asn residues orange, Lys residues blue, and Arg residues turquoise. There is significant sequence similarity across all polo domains; there are 19 hydrophobic positions conserved across all polo domains (coloured green), 13 of which participate in dimerization and 9 of which are pocket and interfacial cleft residues. There are an additional 17 hydrophobic positions conserved within the first or second polo domain (coloured yellow). Positions are identified as conserved if >85% of residues are identical or are hydrophobic in nature. Conserved dimer interface (red arrow z,900 ), pocket (filled circle ●), and cleft (open triangle Δ) positions are indicated. The linker regions between polo domains 1 and 2 are outlined in purple. Species notation is as follows: m=M. musculus, h:=H. sapiens, Dm=D. melanogaster, Dr=Danio rerio, r=Rattus norvegicus, Ce=Caenorhabditis elegans, u=Hemicentrotus pulcherrimus, Tb=Trypansoma brucei, and *=partial EST sequences available only.

FIG. 2. Structure of the Sak polo domain dimer. FIG. 2A, FIG. 2B Ribbons (left) and molecular surface representations (right) of the polo domain homodimer viewed perpendicular (FIG. 2A) and parallel (FIG. 2B) to the two-fold symmetry axis. Secondary structure elements of one or both of the polypeptide chains are labeled. The molecular surface corresponding to hydrophobic side chains (Met, Val, Leu, Ile, Phe,) is coloured green and the amino and carboxy termini are labeled N and C, respectively. The asterisk (*) indicates the position of the Trp 853 side chains. Shown in ball and stick model are the side chains of Lys 906 and Asp 868, which form a tight intermolecular salt interaction on each side of the dimer interface (labeled only on the left side of the dimer). The K906R substitution in polo domain 2 is predicted to form a bidentate salt interaction with Asp 868 and an Asp residue substituted for Val 846 in polo domain 1 (modeled in (a), inset). All ribbon diagrams were generated using RIBBONS [41]. Cross section of the polo domain surface shown in a, reveals a large semi enclosed pocket and interfacial cleft. All molecular surfaces were generated using GRASP [42]. FIG. 2C, Stereo view of the Sak polo domain highlighting representative electron density of the experimental MAD map contoured at 2.0σ. Final model is shown in stick representation. FIG. 2C was generated using O [39].

FIG. 3. The polo domain of Sak can self-associate in vivo but Sak may use several mechanisms for self-association. FIG. 3A, The polo domain of Sak can sell-associate in vivo. NIH 3T3 cells were transfected with Flag₃-tagged polo domain (Flag-Sak_{pb}), Myc-tagged polo domain (Myc-Sak_{pb}), or both, as indicated. Immunoprecipitations were performed using an antibody to FLAG and probed with anti-Myc antibody. Myc-Sak_{pb} coimmunoprecipitated with Flag-Sak_{pb} from cells that were transfected with both constructs, but not those that were singly transfected. Reciprocal immunoprecipitations revealed identical results (data not shown). FIG. 3B, Sak constructs generated for coimmunoprecipitation assays. Numbers indicate the first and last amino acid residues for each construct. The kinase domain and polo domain are illustrated by the hatched and black regions respectively. N-terminal tagged Myc and N-terminal tagged FLAG₃ constructs were generated for each construct. (+) or (−) indicate association or lack of association as observed by coimmunoprecipitations shown in FIG. 3C. FIG. 3C, Full length Sak can dimerize in a polo domain independent manner. NIH 3T3 cells were transfected with the constructs illustrated in FIG. 3B, as indicated. Untransfected and single transfected Myc-tagged controls are shown in lanes 1-5, and double transfected coimmunoprecipitation experiments are shown in lanes 6-11. Immunoblots of the lysates demonstrate that all constructs are expressed. Immunoprecipitations were performed using an anti-FLAG antibody and probed with anti-myc antibody. As shown in lane 6, Myc-tagged Sak coimmunoprecipitated with FLAG₃-tagged Sak, showing that full-length Sak can self associate. Deletion of the polo domain (Sak_{Δpd}) did not abolish this association (lane 7), showing that self-association of full-length Sak does not require the polo domain. A larger C-terminal deletion of an additional 241 residues, Sak_{Δ(pd+}241), did not self associate by coimmunoprecipitation (lane 8). The signal in lane 8, which is larger than the predicted 72 kDa mass for Myc-Sak_{Δpd+}241), is a result of overflow from lane 7. Lanes 9 and 10 illustrate coimmunoprecipitation of the 241 amino acid region, Sak₂₄₁, with Sak_{Δpd+}241) (lane 9) and with itself (lane 10). Myc-tagged Sak₂₄₁ did not coimmunoprecipitate with the polo domain, Sak_{pd} (lane 11). Immunoprecipitation of the single-transfected Myc-tagged constructs with anti-FLAG antibody confirmed that the observed interactions were not due to nonspecific binding of the Myc-tagged constructs (lanes 2-5). The asterisk (*) indicates the positions of α-Myc cross-reactive bands at 21 kDa and 50 kDa.

FIG. 4. Subcellular localization of EGFP-fusion proteins demonstrate that the polo domain of Sak is sufficient for localization. FIG. 4A, FIG. 4C, Localization of EGFP-Sak, EGFP-Sak_{Δpd}, and EGFP-Sak_{pd}. Cells were stained with anti-γ-tubulin or TRITC-phalloidin to indicate the positions of the centrosomes and actin cleavage furrow respectively. EGFP-Sak localizes to centrosomes (FIG. 4A, panel i) and the cleavage furrow (FIG. 4C, panel i). Deletion of the polo domain (Sak_{Δpd}) does not abolish subcellular localization (FIG. 4A, panel ii) and the polo domain itself localizes to centrosomes (FIG. 4A, panel iii) and the cleavage furrow (FIG. 4C, panel ii). Localization of Sak_{Δ(pd+241)}, Sak₂₄₁, and EGFP control are not shown but quantified results are shown in FIG. 4B. FIG. 4B, Bar graph representing the percentage of cells showing centrosomal localization with a sample population of n=100, scored in triplicate.

The present invention will now be described only by way of example, in which reference will be made to the following Tables:

Table 1 shows the data collection, structure determination and refinement statistics for the polo domain of Sak. The following is the legend for Table 1:

¹Numbers in parentheses refer to data for the highest resolution shell (2.00-2.07Å)

²R_{sym}=100×Σ|I−<I>|/Σ<I>, where I is the observed intensity and <I> is the average intensity from multiple observations of symmetry-related reflections.

³Phasing power for isomorphous and anomalous acentric reflections, where phasing power=<[|F_{h,c}|/phase-integrated lack of closure]>.

⁴R_{free} was calculated with 10% of the data.

Table 2 shows the structural coordinates of a polo domain.

In Table 2, from the left, the second column identifies the atom number; the third identifies the atom type; the fourth identifies the amino acid type; the fifth identifies the chain name; the sixth identifies the residue number; the seventh identifies the x coordinates; the eighth identifies the y coordinates; the ninth identifies the z coordinates; the tenth identifies the occupancy; and the eleventh identifies the temperature factor.

### DETAILED DESCRIPTION OF THE INVENTION

Glossary

Unless otherwise indicated, all terms used herein have the same meaning as they would to one skilled in the art of the present invention. Practitioners are particularly directed to Current Protocols in Molecular Biology (Ansubel) for definitions and terms of the art.

“Polo Family Kinase” refers to a member of a family of cell cycle regulators that have been shown to be important for progression through the cell cycle (Lane, H. A., Trends in Cell Biol. 1997, 7:63-68). The family contains the following related but distinct members:
(1) Plk1 (human polo-like kinase) and its homologs Polo (Drosophila), cdc5 (S. cerevisiae), Plx1 (Xenopus), and Plo1 (S. pombe), (see GenBank sequences in Accession No. P53350 (human Plk) [Hamanaka, R., et al, Cell Growth Differ. 5 (3), 249-257 (1994)], No. P52304 (Drosophila Polo) [Llamazares, S et al, Genes Dev. 5 (12A), 2153-2165 (199 )1, No. P32562 (S. cerevisiae cdc5) [Kitada, K., et al, Mol. Cell. Biol. 13 (7), 4445-4457 (1993)], No. AAC60017 (Plx1 Xenopus) [Kumagai, A. and Dunphy, W. G., Science 273 (5280), 1377-1380 (1996)], No. P50528 (S. pombe Plo1) [Ohkura, H., et al, Genes Dev. 9 (9), 1059-1073 (1995)];
(2) Prk (polo-related kinase; human) and its murine homolog Fnk (see GenBank sequences in Accession No. AAC50637 [Li B et al, J. Biol. Chem. 271 (32), 19402-19408 (1996)] and Accession No. AAC52191 [Donohue, P. J., et al, J. Biol. Chem. 270 (17), 10351-10357 (1995));
(3) Snk (serum-inducible kinase; murine) (see GenBank sequence in Accession No. P53351 [Simmons, D. L., Mol. Cell. Biol. 12 (9), 4164-4169 (1992)); and,
(4) Sak (serine threonine kinase) (see GenBank sequences in Accession Nos. CAA73575 (human)[Karn, T., et al, Oncol. Rep. 4, 505-510 (1997)], AAC37648 (murine) [Fode, C., et al, Proc. Natl. Acad. Sci. U.S.A. 91 (14), 6388-6392 (1994)], and AAD19607 (Drosophila).

The polo family kinases are characterized by a kinase domain and one or two conserved sequences in the noncatalytic C-terminal domain i.e. the polo domain.

A polo family kinase may be derivable from a variety of sources, including viruses, bacteria, fungi, plants and animals. In a preferred embodiment a polo family kinase is derivable from a mammal. For example, a polo family kinase may be a human Sak polo family kinase

A polo family kinase in the present invention may be a wild type enzyme, or part thereof, or a mutant, variant or homolog, or part of such an enzyme.

The term “wild type” refers to a polypeptide having a primary amino acid sequence that is identical with the native enzyme (for example, the human enzyme).

The term “mutant” refers to a polypeptide having a primary amino acid sequence which differs from the wild type sequence by one or more amino acid additions, substitutions or deletions. Preferably, the mutant has at least 90% sequence identity with the wild type sequence. Preferably, the mutant has 20 mutations or less over the whole wild-type sequence. More preferably the mutant has 10 mutations or less, most preferably 5 mutations or less over the whole wild-type sequence.

The term “variant” refers to a naturally occurring polypeptide that differs from a wild-type sequence. A variant may be found within the same species (i.e. if there is more than one isoform of the enzyme) or may be found within a different species. Preferably the variant has at least 90% sequence identity with the wild type sequence. Preferably, the variant has 20 mutations or less over the whole wild-type sequence. More preferably, the variant has 10 mutations or less, most preferably 5 mutations or less over the whole wild-type sequence.

The term “part” indicates that the polypeptide comprises a fraction of the wild-type amino acid sequence. It may comprise one or more large contiguous sections of sequence or a plurality of small sections. The “part” may comprise a binding pocket as described herein. The polypeptide may also comprise other elements of sequence, for example, it may be a fusion protein with another protein (such as one which aids isolation or crystallisation of the polypeptide). Preferably the polypeptide comprises at least 50%, more preferably at least 65%, most preferably at least 80% of the wild-type sequence.

The term “homolog” means a polypeptide having a degree of homology with the wild-type amino acid sequence. The term “homology” refers to a degree of complementarity. There may be partial homology or complete homology. A sequence that is “substantially homologous” refers to a partially complementary sequence that at least partially inhibits an identical sequence from hybridizing to a target nucleic acid. Inhibition of hybridization of a completely complementary sequence to the target sequence may be examined using a hybridization assay (e.g. Southern or northern blot, solution hybridization, etc.) under conditions of reduced stringency. A sequence that is substantially homologous or a hybridization probe will compete for and inhibit the binding of a completely homologous sequence to the target sequence under conditions of reduced stringency. However, conditions of reduced stringency can be such that non-specific binding is permitted, as reduced stringency conditions require that the binding of two sequences to one another be a specific (i.e., a selective) interaction. The absence of non-specific binding may be tested using a second target sequence which lacks even a partial degree of complementarity (e.g., less than about 30% homology or identity). The substantially homologous sequence or probe will not hybridize to the second non-complementary target sequence in the absence of non-specific binding.

The phrase “percent identity” or “% identity” refers to the percentage of sequence similarity found in a comparison of two or more amino acid sequences. Percent identity can be determined electronically using conventional programs, e.g., by using the MEGALIGN program (LASERGENE software package, DNASTAR). The MEGALIGN program can create alignments between two or more amino acid sequences according to different methods, e.g., the Clustal Method. (Higgins, D. G. and P. M. Sharp (1988) Gene 73:237-244.) Gaps of low or of no homology between the two amino acid sequences are not included in determining percentage similarity.

In the present context, a homologous sequence is taken to include an amino acid sequence which may have at least 75, 85 or 90% identity, preferably at least 95 or 98% identity to the wild-type sequence. The homologs will comprise the same sites (for example, binding pockets) as the subject amino acid sequence.

A sequence for a polo family kinase or a polo domain or binding pocket thereof may have deletions, insertions or substitutions of amino acid residues which produce a silent change and result in a functionally equivalent enzyme. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The polypeptides may also have a homologous substitution (substitution and replacement are both used herein to mean the interchange of an existing amino acid residue, with an alternative residue) i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

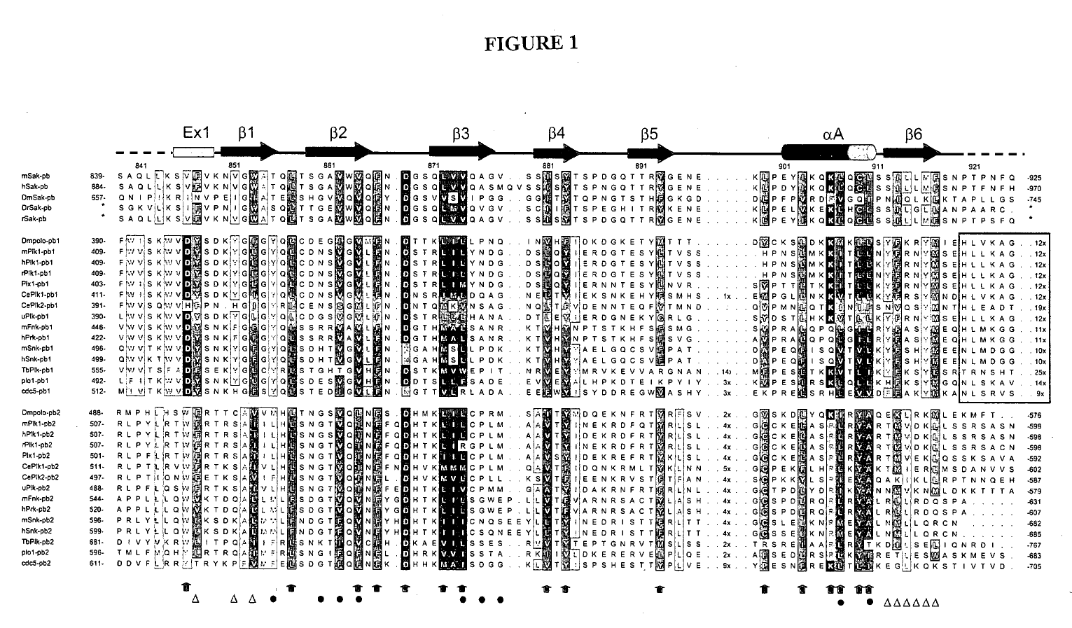

A “polo domain” refers to a domain comprising a polo motif that is a highly conserved sequence in the non-catalytic domain of polo family kinases. FIG. 1 shows the sequences of polo domains from various polo family kinases.

In the present invention the polo domain may be a polo domain of Plk1, Polo, cdc5, Plx, Plo, Prk, Fnk, Snk, or Sak., preferably Sak.

“Binding pocket” refers to a region or site of a polo domain or molecular complex thereof that as a result of its shape, favorably associates with another region of the polo domain or polo family kinase, or with a ligand or a part thereof. For example, it may comprise a region responsible for binding a ligand. In an aspect, a binding pocket comprises a dimeric structure.

A “ligand” refers to a compound or entity that associates with a polo domain or binding pocket thereof including substrates or analogues or parts thereof, effectors, or modulators of polo family kinases, including inhibitors. A ligand may be designed rationally by using a model according to the present invention. For example, a ligand for Plk may be Golgi Reassembly Stacking Protein of 65 kDa (GRASP65) (Lin Cy et al, Proc. Natl. Acad, Sci USA 2000, 7; 97(23): 12589-94), an α, β, or γ-tubulin (Feng, Y et al, Biochem J 1999 15;339 (Pt2): 435-42); human cytomegalovirus (HCMV) pp65 lower matrix protein (Gallina, A. et al J. Virol. 1999 73(2): 1468-78); associated with peptidyl-prolyl isomerase (Pin1), septins [8], Spc72, SMc1, Smc3, IrrI [23], Bfa1 [25], Mid1p [26], cyclin B1, Scc1, Cdc16, Cdc27, MKLP-1, and Hsp90 [reviewed in ref. 1]. A ligand for Prk/Fnk and Snk may be Cib, a Ca²⁺ and integrin-binding protein.

The term “binding pocket” (BP) also includes a homolog of the binding pocket or a portion thereof. As used herein, the term “homolog” in reference to a binding pocket refers to a binding pocket or a portion thereof which may have deletions, insertions or substitutions of amino acid residues as long as the binding specificity is retained. In this regard, deliberate amino acid substitutions may be made on the basis of similarity in polarity, charge, solubility, hydrophobicity, hydrophilicity, and/or the amphipathic nature of the residues as long as the binding specificity of the binding pocket is retained.

As used herein, the term “portion thereof” means the structural coordinates corresponding to a sufficient number of amino acid residues of a binding pocket (or homologs thereof) that are capable of associating with a ligand. For example, the structural coordinates provided in a crystal structure may contain a subset of the amino acid residues in a binding pocket which may be useful in the modelling and design of compounds that bind to the binding pocket.

Crystal

The invention provides crystal structures. As used herein, the term “crystal” or “crystalline” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer. In one aspect, the crystal is usable in X-ray crystallography techniques. Here, the crystals used can withstand exposure to X-ray beams used to produce a diffraction pattern data necessary to solve the X-ray crystallographic structure. A crystal of a polo domain or binding pocket may be characterized as being capable of diffracting x-rays in a pattern defined by one of the crystal forms depicted in Blundel et al 1976, Protein Crystallography, Academic Press.

The invention contemplates a crystal comprising a polo domain or binding pocket thereof of the invention.

In an embodiment, the invention relates to a crystal that is characterized as follows:
(a) dimeric in nature;
(b) comprising a two-sheet, strand-exchange β-fold.

The crystal comprising two monomers (i.e.. a dimer), preferably a crystal of the polo domain of Sak that is dimeric, may be further characterized by one or more of the following properties:
(a) a monomer comprising at its amino terminus five β-strands (β₁-β₅, one α-helix (αA)₁, and a C-terminal β-strand (β₆);
(b) β-strands 6, 1, 2, and 3 from one monomer form a contiguous anti parallel sheet with β-strands 4 and 5 from a second monomer;
(c) two β-sheets pack with a crossing angle of 110°, orienting hydrophobic surfaces inwards and hydrophilic surfaces outwards;
(d) helix αA, which is colinear with β⁻-strand 6 of the same monomer, burying a large portion of the non-overlapping hydrophobic β-sheet surfaces;
(e) interactions involving helices αA comprise a majority of the hydrophobic core structure and also the dimer interface;
(f) a total surface area buried by dimer formation is 2447-2448 Å², preferably 2448 Å²;
(g) the dimeric structure is clam like (60 Å×44 Å×20 Å), hinged at one end through the seamless association of β-strands 3 from each monomer;
(h) a deep cavity of approximate dimensions 17 Å×8-8.5 Å×11.3-12 Å, in particular 17 Å×8 Å×12 Å extending inwards from the mouth of the structure;
(i) an intermolecular salt interaction between Asp 868 and Lys 906; and
(j) a dimer comprising an entranceway to a cavity of (h) above that is relatively small (about 17 Å×7.5 Å) and partitioned in two by the contact of the Trp 853 side chains from each polypeptide of the dimer.

A crystal of the invention may comprise amino acids residues Asp 868 and Lys 906.

Preferably the atoms of the Asp 868 and Lys 906 amino acid residues have the structural coordinates as set out in Table 2.

In an embodiment, a crystal of a polo domain of the invention belongs to space group P3₂12. The term “space group” refers to the lattice and symmetry of the crystal. In a space group designation the capital letter indicates the lattice type and the other symbols represent symmetry operations that can be carried out on the contents of the asymmetric unit without changing its appearance

A crystal of the invention may comprise a unit cell having the following unit dimensions: a=b=51.78 (±0.05) Å, c=146.94 (±0.05) Å. The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

In a preferred embodiment, a crystal of the invention has the structural coordinates as shown in Table 2. As used herein, the term “structural coordinates” refers to a set of values that define the position of one or more amino acid residues with reference to a system of axes. The term refers to a data set that defines the three dimensional structure of a molecule or molecules (e.g. Cartesian coordinates, temperature factors, and occupancies). Structural coordinates can be slightly modified and still render nearly identical three dimensional structures. A measure of a unique set of structural coordinates is the root-mean-square deviation of the resulting structure. Structural coordinates that render three dimensional structures (in particular a three dimensional structure of a ligand binding pocket) that deviate from one another by a root-mean-square deviation of less than 5 Å, 4 Å, 3 Å, 2 Å, or 1.5 Å may be viewed by a person of ordinary skill in the art as very similar.

Variations in structural coordinates may be generated because of mathematical manipulations of the structural coordinates of a polo domain described herein. For example, the structural coordinates of Table 2 may be manipulated by crystallographic permutations of the structural coordinates, fractionalization of the structural coordinates, integer additions or substractions to sets of the structural coordinates, inversion of the structural coordinates or any combination of the above.

Variations in the crystal structure due to mutations, additions, substitutions, and/or deletions of the amino acids, or other changes in any of the components that make up the crystal may also account for modifications in structural coordinates. If such modifications are within an acceptable standard error as compared to the original structural coordinates, the resulting structure may be the same. Therefore, a ligand that bound to a polo domain or binding pocket thereof, would also be expected to bind to another polo domain or binding pocket whose structural coordinates defined a shape that fell within the acceptable error. Such modified structures of a polo domain or binding pocket thereof are also within the scope of the invention.

Various computational analyses may be used to determine whether a molecule or the binding pocket thereof is sufficiently similar to all or parts of a polo domain or binding pocket thereof. Such analyses may be carried out using conventional software applications and methods as described herein.

A crystal of the invention may also be specifically characterised by the parameters, diffraction statistics and/or refinement statistics set out in Table 1.

With reference to a crystal of the present invention, residues in a binding pocket may be defined by their spatial proximity to a ligand in the crystal structure. For example, a binding pocket may be defined by their proximity to a modulator.

A crystal or secondary or three-dimensional structure of a polo domain or binding pocket thereof may be more specifically defined by one or more of the atomic contacts of atomic interactions in the crystal (e.g. between Asp 868 and Lys 906). An atomic interaction can be defined by an atomic contact (more preferably, a specific atom of an amino acid residue where indicated) on the polo domain, and an atomic contact (more preferably, a specific atom of an amino acid residue where indicated) on the polo domain or ligand.

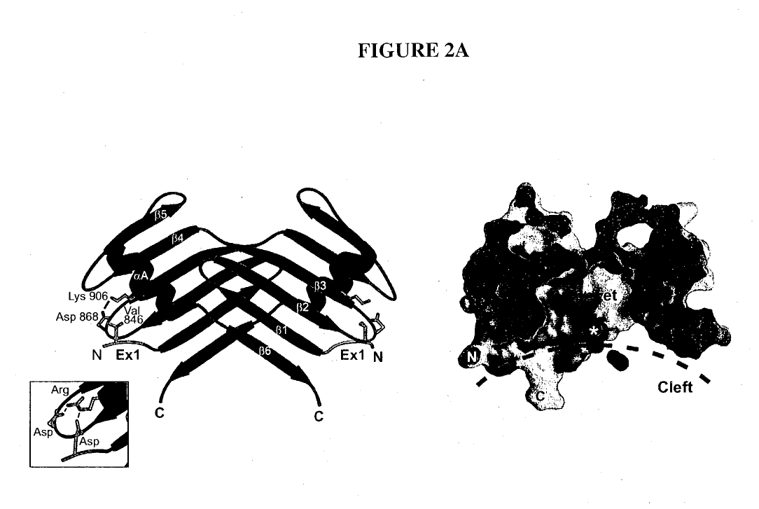

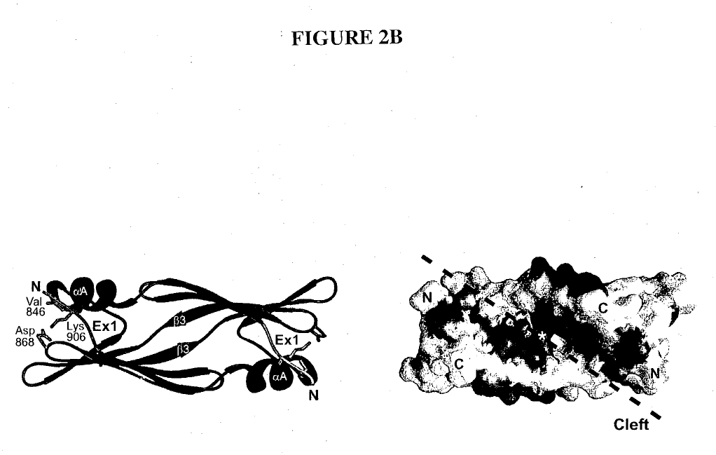

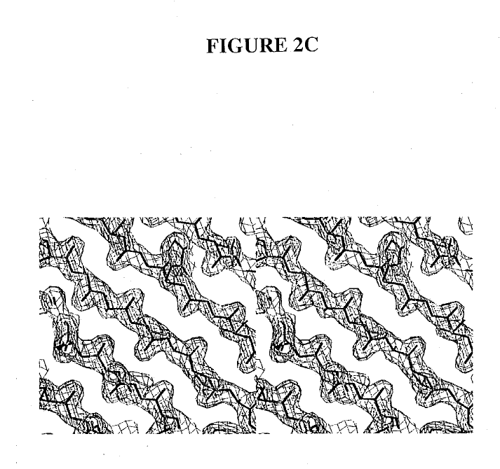

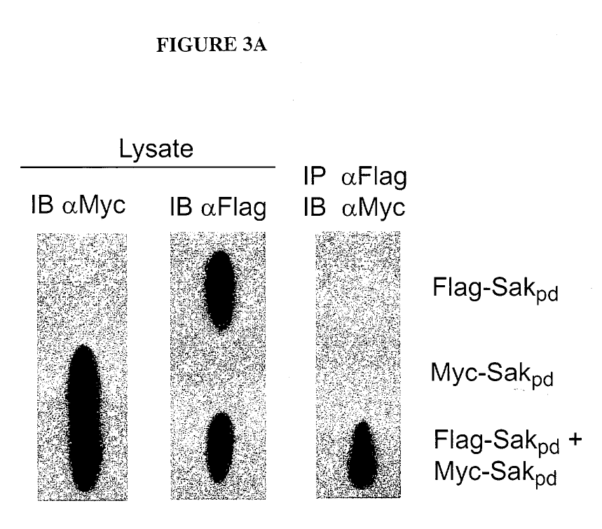

Illustrations of particular crystals of the invention are shown in FIGS. 2A and 2B.

A crystal of the invention includes a polo domain or binding pocket thereof in association with one or more moieties, including heavy-metal atoms i.e. a derivative crystal, or one or more ligands or molecules i.e. a co-crystal.

The term “associate”, “association” or “associating” refers to a condition of proximity between a moiety (i.e. chemical entity or compound or portions or fragments thereof), and a polo domain or binding pocket thereof. The association may be non-covalent i.e. where the juxtaposition is energetically favored by for example, hydrogen-bonding, van der Waals, or electrostatic or hydrophobic interactions, or it may be covalent.

The term “heavy-metal atoms” refers to an atom that can be used to solve an x-ray crystallography phase problem, including but not limited to a transition element, a lanthanide metal, or an actinide-metal. Lanthanide metals include elements with atomic numbers between 57 and 71, inclusive. Actinide metals include elements with atomic numbers between 89 and 103, inclusive.

Multiwavelength anomalous diffraction (MAD) phasing may be used to solve protein structures using selenomethionyl (SeMet) proteins. Therefore, a complex of the invention may comprise a crystalline polo domain or binding pocket with selenium on the methionine residues of the protein.

A crystal may comprise a complex between a polo domain or binding pocket thereof and one or more ligands or molecules. In other words the polo domain or binding pocket may be associated with one or more ligands or molecules in the crystal. The ligand may be any compound that is capable of stably and specifically associating with the polo domain or binding pocket. A ligand may, for example, be a modulator of a polo family kinase or another polo family kinase, in particular a polo domain of another polo family kinase.

In an embodiment of the invention, a binding pocket is in association with a cofactor in the crystal. A “cofactor” refers to a molecule required for enzyme activity and/or stability. For example, the cofactor may be a metal ion.

Therefore, the present invention also provides:
(a) a crystal comprising a polo domain or binding pocket thereof and a substrate or analogue thereof; or
(b) a crystal comprising a polo domain or binding pocket thereof and a ligand.

A structure of a complex of the invention may be defined by selected intermolecular contacts.

A crystal of the invention may enable the determination of structural data for a ligand. In order to be able to derive structural data for a ligand, it is necessary for the molecule to have sufficiently strong electron density to enable a model of the molecule to be built using standard techniques. For example, there should be sufficient electron density to allow a model to be built using XTALVWEW (McRee 1992 J. Mol. Graphics. 10 44-46).

Method of Making a Crystal

The present invention also provides a method of making a crystal according to the invention. The crystal may be formed from an aqueous solution comprising a purified polypeptide comprising a polo domain, in particular a polo family kinase or part or fragment thereof (e.g. a binding pocket). A method may utilize a purified polypeptide comprising a binding pocket to form a crystal. For example, amino acid residues 839 to 925 of murine Sak may be used to prepare a polo domain structure of the invention.

The term “purified” in reference to a polypeptide, does not require absolute purity such as a homogenous preparation rather it represents an indication that the polypeptide is relatively purer than in the natural environment. Generally, a purified polypeptide is substantially free of other proteins, lipids, carbohydrates, or other materials with which it is naturally associated, preferably at a functionally significant level for example at least 85% pure, more preferably at least 95% pure, most preferably at least 99% pure. A skilled artisan can purify a polypeptide comprising using standard techniques for protein purification. A substantially pure polypeptide will yield a single major band on a non-reducing polyacrylamide gel. Purity of the polypeptide can also be determined by amino-terminal amino acid sequence analysis.

A polypeptide used in the method may be chemically synthesized in whole or in part using techniques that are well-known in the art. Alternatively, methods are well known to the skilled artisan to construct expression vectors containing a native or mutated polo family kinase coding sequence and appropriate transcriptional/translational control signals. These methods include in vitro recombinant DNA techniques, synthetic techniques, and in vivo recombination/genetic recombination. See for example the techniques described in Sambrook et al. (Molecular Cloning: A Laboratory Manual, 2nd Edition, Cold Spring Harbor Laboratory press (1989)), and other laboratory textbooks. (See also Sarker et al, Glycoconjugate J. 7:380, 1990; Sarker et al, Proc. Natl. Acad, Sci. USA 88:234-238, 1991, Sarker et al, Glycoconjugate J. 11: 204-209, 1994; Hull et al, Biochem Biophys Res Commun 176:608, 1991 and Pownall et al, Genomics 12:699-704, 1992).

Crystals may be grown from an aqueous solution containing the purified polypeptide by a variety of conventional processes. These processes include batch, liquid, bridge, dialysis, vapor diffusion, and hanging drop methods. (See for example, McPherson, 1982 John Wiley, New York; McPherson, 1990, Eur. J. Biochem. 189: 1-23; Webber. 1991, Adv. Protein Chem. 41:1-36). Generally, native crystals of the invention are grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

Derivative crystals of the invention can be obtained by soaking native crystals in a solution containing salts of heavy metal atoms. A complex of the invention can be obtained by soaking a native crystal in a solution containing a compound that binds the polypeptide, or they can be obtained by co-crystallizing the polypeptide in the presence of one or more compounds. In order to obtain co-crystals with a compound which binds deep within the tertiary structure of the polypeptide it is necessary to use the second method.

Once the crystal is grown it can be placed in a glass capillary tube and mounted onto a holding device connected to an X-ray generator and an X-ray detection device. Collection of X-ray diffraction patterns are well documented by those skilled in the art (See for example, Ducruix and Geige, 1992, IRL Press, Oxford, England). A beam of X-rays enter the crystal and diffract from the crystal. An X-ray detection device can be utilized to record the diffraction patterns emanating from the crystal. Suitable devices include the Marr 345 imaging plate detector system with an RU200 rotating anode generator.

Multiwavelength anomalous diffraction (MAD) phasing using selenomethionyl (SeMet) proteins may be used to determine a crystal of the invention. Thus, the invention contemplates a method for determining a crystal structure of the invention using a selenomethionyl derivative of a polo domain or a binding pocket thereof.

Methods for obtaining the three dimensional structure of the crystalline form of a molecule or complex are described herein and known to those skilled in the art (see Ducruix and Geige 1992, IRL Press, Oxford, England). Generally, the x-ray crystal structure is given by the diffraction patterns. Each diffraction pattern reflection is characterized as a vector and the data collected at this stage determines the amplitude of each vector. The phases of the vectors may be determined by the isomorphous replacement method where heavy atoms soaked into the crystal are used as reference points in the X-ray analysis (see for example, Otwinowski, 1991, Daresbury, United Kingdom, 80-86). The phases of the vectors may also be determined by molecular replacement (see for example, Naraza, 1994, Proteins 11:281-296). The amplitudes and phases of vectors from the crystalline form are determined in accordance with these methods can be used to analyze other related crystalline polypeptides.

The unit cell dimensions and symmetry, and vector amplitude and phase information can be used in a Fourier transform function to calculate the electron density in the unit cell i.e. to generate an experimental electron density map. This may be accomplished using the PHASES package (Furey, 1990). Amino acid sequence structures are fit to the experimental electron density map (i.e. model building) using computer programs (e.g. Jones, T A. et al, Acta Crystallogr A47, 100-119, 1991). This structure can also be used to calculate a theoretical electron density map. The theoretical and experimental electron density maps can be compared and the agreement between the maps can be described by a parameter referred to as R-factor. A high degree of overlap in the maps is represented by a low value R-factor. The R-factor can be minimized by using computer programs that refine the structure to achieve agreement between the theoretical and observed electron density map. For example, the XPLOR program, developed by Brunger (1992, Nature 355:472-475) can be used for model refinement.

A three dimensional structure of the molecule or complex may be described by atoms that fit the theoretical electron density characterized by a minimum R value. Files can be created for the structure that defines each atom by coordinates in three dimensions.

Model

A crystal structure of the present invention may be used to make a model of a polo domain or binding pocket thereof. A model may, for example, be a structural model or a computer model. A model may represent the secondary, tertiary and/or quaternary structure of the binding pocket. The model itself may be in two or three dimensions. It is possible for a computer model to be in three dimensions despite the constraints imposed by a conventional computer screen, if it is possible to scroll along at least a pair of axes, causing “rotation” of the image.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

Preferably, modelling is performed using a computer and may be further optimized using known methods. This is called modelling optimisation.

An integral step to an approach of the invention for designing modulators of a subject polo domain involves construction of computer graphics models of the domain which can be used to design pharmacophores by rational drug design. For instance, for a modulator to interact optimally with the subject domain, it will generally be desirable that it have a shape which is at least partly complimentary to that of a particular binding pocket of the domain, as for example those portions of the domain which are involved in recognition of a ligand. Additionally, other factors, including electrostatic interactions, hydrogen bonding, hydrophobic interactions, desolvation effects, and cooperative motions of ligand and domain, all influence the binding effect and should be taken into account in attempts to design bioactive modulators.

As described herein, a computer-generated molecular model of the subject polo domain can be created. In preferred embodiments, at least the Cα-carbon positions of the polo domain sequence of interest are mapped to a particular coordinate pattern, such as the coordinates for a polo domain shown in Table 2, by homology modeling, and the structure of the protein and velocities of each atom are calculated at a simulation temperature (Tₒ) at which the docking simulation is to be determined. Typically, such a protocol involves primarily the prediction of side-chain conformations in the modeled domain, while assuming a main-chain trace taken from a tertiary structure such as provided in Table 2 and the Figures. Computer programs for performing energy minimization routines are commonly used to generate molecular models. For example, both the CHARMM (Brooks et al. (1983) J Comput Chem 4:187-217) and AMBER (Weiner et al (1981) J. Comput. Chem. 106: 765) algorithms handle all of the molecular system setup, force field calculation, and analysis (see also, Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488). At the heart of these programs is a set of subroutines that, given the position of every atom in the model, calculate the total potential energy of the system and the force on each atom. These programs may utilize a starting set of atomic coordinates, such as the coordinates provided in Table 2, the parameters for the various terms of the potential energy function, and a description of the molecular topology (the covalent structure). Common features of such molecular modeling methods include: provisions for handling hydrogen bonds and other constraint forces; the use of periodic boundary conditions; and provisions for occasionally adjusting positions, velocities, or other parameters in order to maintain or change temperature, pressure, volume, forces of constraint, or other externally controlled conditions.

Most conventional energy minimization methods use the input data described above and the fact that the potential energy function is an explicit, differentiable function of Cartesian coordinates, to calculate the potential energy and its gradient (which gives the force on each atom) for any set of atomic positions. This information can be used to generate a new set of coordinates in an effort to reduce the total potential energy and, by repeating this process over and over, to optimize the molecular structure under a given set of external conditions. These energy minimization methods are routinely applied to molecules similar to the subject polo domain.

In general, energy minimization methods can be carried out for a given temperature, Tᵢ, which may be different than the docking simulation temperature, Tₒ. Upon energy minimization of the molecule at Tᵢ, coordinates and velocities of all the atoms in the system are computed. Additionally, the normal modes of the system are calculated. It will be appreciated by those skilled in the art that each normal mode is a collective, periodic notion, with all parts of the system moving in phase with each other, and that the motion of the molecule is the superposition of all normal modes. For a given temperature, the mean square amplitude of motion in a particular mode is inversely proportional to the effective force constant for that mode, so that the motion of the molecule will often be dominated by the low frequency vibrations.

After the molecular model has been energy minimized at Tᵢ, the system is “heated” or “cooled” to the simulation temperature, Tₒ, by carrying out an equilibration run where the velocities of the atoms are scaled in a step-wise manner until the desired temperature, Tₒ, is reached. The system is further equilibrated for a specified period of time until certain properties of the system, such as average kinetic energy, remain constant. The coordinates and velocities of each atom are then obtained from the equilibrated system.

Further energy minimization routines can also be carried out. For example, a second class of methods involves calculating approximate solutions to the constrained EOM for the protein. These methods use an iterative approach to solve for the Lagrange multipliers and, typically, only need a few iterations if the corrections required are small. The most popular method of this type, SHAKE (Ryckaert et al. (1977) J Comput Phys 23:327; and Van Gunsteren et al. (1977) Mol Phys 34:1311) is easy to implement and scales as O(N) as the number of constraints increases. Therefore, the method is applicable to molecules such as the polo domains of the present invention. An alternative method, RATTLE (Anderson (1983) J Comput Phys 52:24) is based on the velocity version of the Verlet algorithm. Like SHAKE, RATTLE is an iterative algorithm and can be used to energy minimize the model of the subject protein.

Overlays and super positioning with a three dimensional model of a polo domain or binding pocket thereof of the invention may be used for modelling optimisation. Additionally alignment and/or modelling can be used as a guide for the placement of mutations on a polo domain or binding pocket thereof to characterize the nature of the site in the context of a cell.

The three dimensional structure of a new crystal may be modelled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are X-PLOR (Brunger 1992, Nature 355: 472-475), AMoRE (Navaza, 1994, Acta Crystallogr. A50:157-163), the CCP4 package (Collaborative Computational Project, Number 4, “The CCP4 Suite: Programs for Protein Crystallography”, Acta Cryst., Vol. D50, pp. 760-763, 1994), the MERLOT package (P. M. D. Fitzgerald, J. Appl. Cryst., Vol. 21, pp. 273-278, 1988) and XTALVIEW (McCree et al (1992) J. Mol. Graphics 10: 44-46. It is preferable that the resulting structure not exhibit a root-mean-square deviation of more than 3 Å.

Molecular replacement computer programs generally involve the following steps: (1) determining the number of molecules in the unit cell and defining the angles between them (self rotation function); (2) rotating the known structure against diffraction data to define the orientation of the molecules in the unit cell (rotation function); (3) translating the known structure in three dimensions to correctly position the molecules in the unit cell (translation function); (4) determining the phases of the X-ray diffraction data and calculating an R-factor calculated from the reference data set and from the new data wherein an R-factor between 30-50% indicates that the orientations of the atoms in the unit cell have been reasonably determined by the method; and (5) optionally, decreasing the R-factor to about 20% by refining the new electron density map using iterative refinement techniques known to those skilled in the art (refinement).

The quality of the model may be analysed using a program such as PROCHECK or 3D-Profiler [Laskowski et al 1993 J. Appl. Cryst. 26:283-291; Luthy R. et al, Nature 356: 83-85, 1992; and Bowie, J. U. et al, Science 253: 164-170, 1991]. Once any irregularities have been resolved, the entire structure may be further refined.

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structural coordinates of crystals provided by the invention, molecular modelling may be used to determine the structural coordinates of a crystalline mutant or homolog of a polo domain or binding pocket thereof. By the same token a crystal of the invention can be used to provide a model of a ligand. Modelling techniques can then be used to approximate the three dimensional structure of ligand derivatives and other components which may be able to mimic the atomic contacts between a ligand and polo domain or binding pocket.

Computer Format of Crystals/Models

Information derivable from a crystal of the present invention (for example the structural coordinates) and/or the model of the present invention may be provided in a computer-readable format.

Therefore, the invention provides a computer readable medium or a machine readable storage medium which comprises the structural coordinates of a polo domain or binding pocket thereof including all or any parts thereof, or ligands including portions thereof. Such storage medium or storage medium encoded with these data are capable of displaying on a computer screen or similar viewing device, a three-dimensional graphical representation of a molecule or molecular complex which comprises such polo domain, binding pockets or similarly shaped homologous domains or binding pockets. Thus, the invention also provides computerized representations of the secondary or three-dimensional structures of a polo domain or binding pocket of the invention, including any electronic, magnetic, or electromagnetic storage forms of the data needed to define the structures such that the data will be computer readable for purposes of display and/or manipulation.

In an aspect the invention provides a computer for producing a three-dimensional representation of a molecule or molecular complex, wherein said molecule or molecular complex comprises a polo domain or binding pocket thereof defined by structural coordinates of a polo domain or binding pocket or structural coordinates of atoms of a ligand, or a three-dimensional representation of a homologue of said molecule or molecular complex, wherein said homologue comprises a polo domain, binding pocket or ligand that has a root mean square deviation from the backbone atoms not more than 1.5 angstroms wherein said computer comprises:
(a) a machine-readable data storage medium comprising a data storage material encoded with machine readable data wherein said data comprises the structural coordinates of a polo domain or binding pocket thereof or a ligand according to Table 2;
(b) a working memory for storing instructions for processing said machine-readable data;
(c) a central-processing unit coupled to said working memory and to said machine-readable data storage medium for processing said machine readable data into said three-dimensional representation; and
(d) a display coupled to said central-processing unit for displaying said three-dimensional representation.

The invention also provides a computer for determining at least a portion of the structural coordinates corresponding to an X-ray diffraction pattern of a molecule or molecular complex wherein said computer comprises:
(a) a machine-readable data storage medium comprising a data storage material encoded with machine readable data wherein said data comprises the structure coordinates according to Table 2;
(b) a machine-readable data storage medium comprising a data storage material encoded with machine readable data wherein said data comprises an X-ray diffraction pattern of said molecule or molecular complex;
(c) a working memory for storing instructions for processing said machine-readable data of (a) and (b);
(d) a central-processing unit coupled to said working memory and to said machine-readable data storage medium of (a) and (b) for performing a Fourier transform of the machine readable data of (a) and for processing said machine readable data of (b) into structural coordinates; and
(e) a display coupled to said central-processing unit for displaying said structural coordinates of said molecule or molecular complex.
   Structural Studies

The present invention also provides a method for determining the secondary and/or tertiary structures of a polo domain or part thereof by using a crystal, or a model according to the present invention. The domain or part thereof may be any domain or part thereof for which the secondary and or tertiary structure is uncharacterised or incompletely characterised. In a preferred embodiment the domain shares (or is predicted to share) some structural or functional homology to a crystal of the present invention. For example, the domain may show a degree of structural homology over some or all parts of the primary amino acid sequence.

The polo domain may be a polo domain of a polo family kinase with a different specificity for a ligand or substrate. Alternatively (or in addition) the domain may be a polo domain from a different species.

The domain may be from a mutant of a wild-type polo family kinase, in particular Plk1 or Sak. A mutant may arise naturally, or may be made artificially (for example using molecular biology techniques). The mutant may also not be “made” at all in the conventional sense, but merely tested theoretically using the model of the present invention. A mutant may or may not be functional.

Thus, using the model of the present invention, the effect of a particular mutation on the overall two and/or three dimensional structure of a polo domain and/or the interaction between a binding pocket of the enzyme and a ligand can be investigated.

Alternatively, the domain may perform an analogous function or be suspected to show a similar mechanism to a polo domain of a polo family kinase.

The domain may also be the same as the polo domain of the crystal, but in association with a different ligand (for example, modulator or inhibitor) or cofactor. In this way it is possible to investigate the effect of altering the ligand or compound with which the polo domain is associated on the structure of the binding pocket.

Secondary or tertiary structure may be determined by applying the structural coordinates of the crystal or model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a sequence based on the structures of known proteins (i.e. a polo domain of the crystal of the invention). The method utilizes a computer model of the crystal of the present invention (the “known structure”), a computer representation of the amino acid sequence of the domain with an unknown structure, and standard computer representations of the structures of amino acids. The method in particular comprises the steps of; (a) identifying structurally conserved and variable regions in the known structure; (b) aligning the amino acid sequences of the known structure and unknown structure (c) generating co-ordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http://biochem.vt.edu/coul-ses/modeling/homology.htn). Computer programs that can be used in homology modelling are Quanta and the Homology module in the Insight II modelling package distributed by Molecular Simulations Inc, or MODELLER (Rockefeller University, www.iucr.ac.uk/sinris-top/logical/prg-modeller.html).

In step (a) of the homology modelling method, a known structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites (e.g. nucleotide binding sites). The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442-453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoff et al., Meth. Enzymol. 91: 524-545, 1983), and BLOSUM (Henikoff and Henikoff, Proc. Nat. Acad. Sci. USA 89: 10915-'0919, 1992), and the matrices based on alignments derived from three-dimensional structures including that of Johnson and Overington (JO matrices) (J. Mol. Biol. 233: 716-738, 1993).

Alignment based solely on sequence may be used; however, other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating coordinates for the unknown structure, main chain atoms and side chain atoms, both in SCRs and VRs need to be modelled. A variety of approaches known to those skilled in the art may be used to assign co-ordinates to the unknown. In particular, the coordinates of the main chain atoms of SCRs will be transferred to the unknown structure. VRs correspond most often to the loops on the surface of the polypeptide and if a loop in the known structure is a good model for the unknown, then the main chain co-ordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent *minimizations. Molecular dynamics may also be applied in conjunction with energy minimization.

Molecular replacement involves applying a known structure to solve the X-ray crystallographic data set of a polypeptide of unknown structure. The method can be used to define the phases describing the X-ray diffraction data of a polypeptide of unknown structure when only the amplitudes are known. Thus in an embodiment of the invention, a method is provided for determining three dimensional structures of polypeptides with unknown structure by applying the structural coordinates of a crystal of the present invention to provide an X-ray crystallographic data set for a polypeptide of unknown structure, and (b) determining a low energy conformation of the resulting structure.

The structural coordinates of the crystal of the present invention may be applied to nuclear magnetic resonance (NMR) data to determine the three dimensional structures of polypeptides with uncharacterised or incompletely characterised structure. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of domains with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

Screening Method

Another aspect of the present invention concerns molecular models, in particular three-dimensional molecular models of polo domains, and their use as templates for the design of agents able to mimic or inhibit the activity of a polypeptide comprising a polo domain.

In certain embodiments, the present invention provides a method of screening for a ligand that associates with a polo domain or binding pocket and/or modulates the function of a polo family kinase by using a crystal or a model according to the present invention. The method may involve investigating whether a test compound is capable of associating with or binding a polo domain or binding pocket thereof, and/or inhibiting or enhancing interactions of atomic contacts in a polo domain or binding pocket thereof.

In accordance with an aspect of the present invention, a method is provided for screening for a ligand capable of binding to a polo domain or a binding pocket thereof, wherein the method comprises using a crystal or model according to the invention.

In another aspect, the invention relates to a method of screening for a ligand capable of binding to a polo domain or binding pocket thereof, wherein the polo domain or binding pocket thereof is defined by the structural coordinates given herein, the method comprising contacting the polo domain or binding pocket thereof with a test compound and determining if the test compound binds to the polo domain or binding pocket thereof.

In one embodiment, the present invention provides a method of screening for a test compound capable of interacting with one or more key amino acid residues of a binding pocket of a polo domain.

Another aspect of the invention provides a process comprising the steps of:
(a) performing a method of screening for a ligand described above;
(b) identifying one or more ligands capable of binding to a binding pocket; and
(c) preparing a quantity of said one or more ligands.

A further aspect of the invention provides a process comprising the steps of;
(a) performing a method of screening for a ligand as described above;
(b) identifying one or more ligands capable of binding to a binding pocket; and
(c) preparing a pharmaceutical composition comprising said one or more ligands.

Once a test compound capable of interacting with one or more key amino acid residues in a binding pocket of a polo domain has been identified, further steps may be carried out either to select and/or modify compounds and/or to modify existing compounds, to modulate the interaction with the key amino acid residues in the binding pocket.

Yet another aspect of the invention provides a process comprising the steps of;
(a) performing the method of screening for a ligand as described above;
(b) identifying one or more ligands capable of binding to a binding pocket;
(c) modifying said one or more ligands capable of binding to a binding pocket;
(d) performing said method of screening for a ligand as described above; and
(e) optionally preparing a pharmaceutical composition comprising said one or more ligands.

As used herein, the term “test compound” means any compound which is potentially capable of associating with a binding pocket, and/or inhibiting or enhancing interactions of atomic contacts in a binding pocket. If, after testing, it is determined that the test compound does bind to the binding pocket and/or inhibits or enhances interactions of atomic contacts in a binding contact, it is known as a “ligand”.

The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptides synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The increasing availability of biomacromolecule structures of potential pharmacophoric molecules that have been solved crystallographically has prompted the development of a variety of direct computational methods for molecular design, in which the steric and electronic properties of substrate binding sites are use to guide the design of potential ligands (Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153). Directed methods generally fall into two categories: (1) design by analogy in which 3-D structures of known molecules (such as from a crystallographic database) are docked to the domain structure and scored for goodness-of-fit; and (2) de novo design, in which the ligand model is constructed piece-wise in the domain structure. The latter approach, in particular, can facilitate the development of novel molecules, uniquely designed to bind to the subject domain.

The test compound may be screened as part of a library or a data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may tested for their capacity to fit spatially into a binding pocket. As used herein, the term “fits spatially” means that the three-dimensional structure of the test compound is accommodated geometrically in a cavity of a binding pocket. The test compound can then be considered to be a ligand.

A favourable geometric fit occurs when the surface area of the test compound is in close proximity with the surface area of the cavity of a binding pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding pocket.

If a model of the present invention is a computer model, the test compounds may be positioned in a binding pocket through computational docking. If, on the other hand, the model of the present invention is a structural model, the test compounds may be positioned in the binding pocket by, for example, manual docking.

As used herein the term “docking” refers to a process of placing a compound in close proximity with a binding pocket, or a process of finding low energy conformations of a test compound/binding pocket complex.

In an illustrative embodiment, the design of potential polo domain ligands begins from the general perspective of shape complimentary for an active site and substrate specificity subsites of the domain, and a search algorithm is employed which is capable of scanning a database of small molecules of known three-dimensional structure for candidates which fit geometrically into the target protein site. It is not expected that the molecules found in the shape search will necessarily be leads themselves, since no evaluation of chemical interaction necessarily be made during the initial search. Rather, it is anticipated that such candidates might act as the framework for further design, providing molecular skeletons to which appropriate atomic replacements can be made. Of course, the chemical complementarity of these molecules can be evaluated, but it is expected that atom types will be changed to maximize the electrostatic, hydrogen bonding, and hydrophobic interactions with the protein. Most algorithms of this type provide a method for finding a wide assortment of chemical structures that are complementary to the shape of a binding pocket of the subject domain. Each of a set of small molecules from a particular data-base, such as the Cambridge Crystallographic Data Bank (CCDB) (Allen et al. (1973) J. Chem. Doc. 13: 119), is individually docked to the binding pocket or site of a polo domain, in particular a Sak or Plk polo domain, in a number of geometrically permissible orientations with use of a docking algorithm. In a preferred embodiment, a set of computer algorithms called DOCK, can be used to characterize the shape of invaginations and grooves that form active sites and recognition surfaces of a subject structure (Kuntz et al. (1982) J. Mol. Biol 161: 269-288). The program can also search a database of small molecules for templates whose shapes are complementary to particular binding pockets or sites of a structure (DesJarlais et al. (1988) J Med Chem 31: 722-729). These templates normally require modification to achieve good chemical and electrostatic interactions (DesJarlais et al. (1989) ACS Symp Ser 413: 60-69). However, the program has been shown to position accurately known cofactors for ligands based on shape constraints alone.

The orientations are evaluated for goodness-of-fit and the best are kept for further examination using molecular mechanics programs, such as AMBER or CHARMM. Such algorithms have previously proven successful in finding a variety of molecules that are complementary in shape to a given binding site of a structure, and have been shown to have several attractive features. First, such algorithms can retrieve a remarkable diversity of molecular architectures. Second, the best structures have, in previous applications to other proteins, demonstrated impressive shape complementarity over an extended surface area. Third, the overall approach appears to be quite robust with respect to small uncertainties in positioning of the candidate atoms.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) on the molecular surface of the binding site. GRID hence provides a tool for suggesting modifications to known ligands that might enhance binding. It may be anticipated that some of the sites discerned by GRID as regions of high affinity correspond to “pharmacophoric patterns” determined inferentially from a series of known ligands. As used herein, a pharmacophoric pattern is a geometric arrangement of features of the anticipated ligand that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20); however, the constraint of steric and “chemical” fit in the putative (and possibly unknown) binding pocket or site is ignored. Goodsell and Olson (1990, Proteins: Struct Funct Genet 8:195-202) have used the Metropolis (simulated annealing) algorithm to dock a single known ligand into a target protein. They allow torsional flexibility in the ligand and use GRID interaction energy maps as rapid lookup tables for computing approximate interaction energies. Given the large number of degrees of freedom available to the ligand, the Metropolis algorithm is time-consuming and is unsuited to searching a candidate database of a few thousand small molecules.

Yet a further embodiment of the present invention utilizes a computer algorithm such as CLIX which searches such databases as CCDB for small molecules which can be oriented in a binding pocket or site in a way that is both sterically acceptable and has a high likelihood of achieving favorable chemical interactions between the candidate molecule and the surrounding amino acid residues. The method is based on characterizing a binding pocket in terms of an ensemble of favorable binding positions for different chemical groups and then searching for orientations of the candidate molecules that cause maximum spatial coincidence of individual candidate chemical groups with members of the ensemble. The current availability of computer power dictates that a computer-based search for novel ligands follows a breadth-first strategy. A breadth-first strategy aims to reduce progressively the size of the potential candidate search space by the application of increasingly stringent criteria, as opposed to a depth-first strategy wherein a maximally detailed analysis of one candidate is performed before proceeding to the next. CLIX conforms to this strategy in that its analysis of binding is rudimentary—it seeks to satisfy the necessary conditions of steric fit and of having individual groups in “correct” places for bonding, without imposing the sufficient condition that favorable bonding interactions actually occur. A ranked “shortlist” of molecules, in their favored orientations, is produced which can then be examined on a molecule-by-molecule basis, using computer graphics and more sophisticated molecular modeling techniques. CLIX is also capable of suggesting changes to the substituent chemical groups of the candidate molecules that might enhance binding.

The algorithmic details of CLIX is described in Lawerence et al. (1992) Proteins 12:31-41, and the CLIX algorithm can be summarized as follows. The GRID program is used to determine discrete favorable interaction positions (termed target sites) in the binding pocket or site of the protein for a wide variety of representative chemical groups. For each candidate ligand in the CCDB an exhaustive attempt is made to make coincident, in a spatial sense in the binding site of the protein, a pair of the candidate's substituent chemical groups with a pair of corresponding favorable interaction sites proposed by GRID. All possible combinations of pairs of ligand groups with pairs of GRID sites are considered during this procedure. Upon locating such coincidence, the program rotates the candidate ligand about the two pairs of groups and checks for steric hindrance and coincidence of other candidate atomic groups with appropriate target sites. Particular candidate/orientation combinations that are good geometric fits in the binding site and show sufficient coincidence of atomic groups with GRID sites are retained.

Consistent with the breadth-first strategy, this approach involves simplifying assumptions. Rigid protein and small molecule geometry is maintained throughout. As a first approximation rigid geometry is acceptable as the energy minimized coordinates of a polo domain, in particular a Sak polo domain deduced structure, as described herein, describe an energy minimum for the molecule, albeit a local one. If the surface residues of the site of interest are not involved in crystal contacts then the crystal configuration of those residues is used merely as a starting point for energy minimization, and potential solution structures for those residues determined. The deduced structure described herein should reasonably mimic the mean solution configuration.

A further assumption implicit in CLIX is that the potential ligand, when introduced into the binding pocket or site, does not induce change in the protein's stereochemistry or partial charge distribution and so alter the basis on which the GRID interaction energy maps were computed. It must also be stressed that the interaction sites predicted by GRID are used in a positional and type sense only, i.e., when a candidate atomic group is placed at a site predicted as favorable by GRID, no check is made to ensure that the bond geometry, the state of protonation, or the partial charge distribution favors a strong interaction between the protein and that group. Such detailed analysis should form part of more advanced modeling of candidates identified in the CLIX shortlist.

Yet another embodiment of a computer-assisted molecular design method for identifying ligands of a polo domain comprises the de novo synthesis of potential ligands by algorithmic connection of small molecular fragments that will exhibit the desired structural and electrostatic complementarity with a polo domain or binding pocket thereof. The methodology employs a large template set of small molecules with are iteratively pieced together in a model of a polo domain or binding pocket. Each stage of ligand growth is evaluated according to a molecular mechanics-based energy function, which considers van der Waals and coulombic interactions, internal strain energy of the lengthening ligand, and desolvation of both ligand and domain. The search space can be managed by use of a data tree which is kept under control by pruning according to the binding criteria.

In an illustrative embodiment, the search space is limited to consider only amino acids and amino acid analogs as the molecular building blocks. Such a methodology generally employs a large template set of amino acid conformations, though need not be restricted to just the 20 natural amino acids, as it can easily be extended to include other related fragments of interest to the medicinal chemist, e.g. amino acid analogs. The putative ligands that result from this construction method are peptides and peptide-like compounds rather than the small organic molecules that are typically the goal of drug design research. The appeal of the peptide building approach is not that peptides are preferable to organics as potential pharmaceutical agents, but rather that: (1) they can be generated relatively rapidly de novo; (2) their energetics can be studied by well-parameterized force field methods; (3) they are much easier to synthesize than are most organics; and (4) they can be used in a variety of ways, for peptidomimetic ligand design, protein-protein binding studies, and even as shape templates in the more commonly used 3D organic database search approach described above.

Such a de novo peptide design method has been incorporated in a software package called GROW (Moon et al. (1991) Proteins 11:314-328). In a typical design session, standard interactive graphical modeling methods are employed to define the structural environment in which GROW is to operate. For instance, environment could be an active site binding pocket of a polo domain, in particular a Sak or Plk polo domain, or it could be a set of features on the protein's surface to which the user wishes to bind a peptide-like molecule. The GROW program then operates to generate a set of potential ligand molecules. Interactive modeling methods then come into play again, for examination of the resulting molecules, and for selection of one or more of them for further refinement.

To illustrate, GROW operates on an atomic coordinate file generated by the user in the interactive modeling session, such as the coordinates provided in Table 2 plus a small fragment (e.g., an acetyl group) positioned in the active site to provide a starting point for peptide growth. These are referred to as “site” atoms and “seed” atoms, respectively. A second file provided by the user contains a number of control parameters to guide the peptide growth (Moon et al. (1991) Proteins 11:314-328).

The operation of the GROW algorithm is conceptually fairly simple. GROW proceeds in an iterative fashion, to systematically attach to the seed fragment each amino acid template in a large preconstructed library of amino acid conformations. When a template has been attached, it is scored for goodness-of-fit to the polo domain or binding pocket thereof, and then the next template in the library is attached to the seed. After all the templates have been tested, only the highest scoring ones are retained for the next level of growth. This procedure is repeated for the second growth level; each library template is attached in turn to each of the bonded seed/amino acid molecules that were retained from the first step, and is then scored. Again, only the best of the bonded seed/dipeptide molecules that result are retained for the third level of growth. The growth of peptides can proceed in the N-to-C direction only, the reverse direction only, or in alternating directions, depending on the initial control specifications supplied by the user. Successive growth levels therefore generate peptides that are lengthened by one residue. The procedure terminates when the user-defined peptide length has been reached, at which point the user can select from the constructed peptides those to be studied further. The resulting data provided by the GROW procedure includes not only residue sequences and scores, but also atomic coordinates of the peptides, related directly to the coordinate system of the domain site atoms.

In yet another embodiment, potential pharmacophoric compounds can be determined using a method based on an energy minimization-quenched molecular dynamics algorithm for determining energetically favorable positions of functional groups in the binding pockets of the subject polo domain. The method can aid in the design of molecules that incorporate such functional groups by modification of known ligands or de novo construction.

For example, the multiple copy simultaneous search method (MCSS) described by Miranker et al. (1991) Proteins 11: 29-34. To determine and characterize a local minima of a functional group in the forcefield of the protein, multiple copies of selected functional groups are first distributed in a binding pocket of interest on the polo domain. Energy minimization of these copies by molecular mechanics or quenched dynamics yields the distinct local minima. The neighborhood of these minima can then be explored by a grid search or by constrained minimization. In one embodiment, the MCSS method uses the classical time dependent Hartee (TDH) approximation to simultaneously minimize or quench many identical groups in the forcefield of the protein.

Implementation of the MCSS algorithm requires a choice of functional groups and a molecular mechanics model for each of them. Groups must be simple enough to be easily characterized and manipulated (3-6 atoms, few or no dihedral degrees of freedom), yet complex enough to approximate the steric and electrostatic interactions that the functional group would have in binding to the pocket or site of interest in the polo domain. A preferred set is, for example, one in which most organic molecules can be described as a collection of such groups (Patai's Guide to the Chemistry of Functional Groups, ed. S. Patai (New York: John Wiley, and Sons, (1989)). This includes fragments such as acetonitrile, methanol, acetate, methyl ammonium, dimethyl ether, methane, and acetaldehyde.

Determination of the local energy minima in the binding pocket or site requires that many starting positions be sampled. This can be achieved by distributing, for example, 1,000-5,000 groups at random inside a sphere centered on the binding site; only the space not occupied by the protein needs to be considered. If the interaction energy of a particular group at a certain location with the protein is more positive than a given cut-off (e.g. 5.0 kcal/mole) the group is discarded from that site. Given the set of starting positions, all the fragments are minimized simultaneously by use of the TDH approximation (Elber et al. (1990) J Am Chem Soc 112: 9161-9175). In this method, the forces on each fragment consist of its internal forces and those due to the protein. The essential element of this method is that the interactions between the fragments are omitted and the forces on the protein are normalized to those due to a single fragment. In this way simultaneous minimization or dynamics of any number of functional groups in the field of a single protein can be performed.

Minimization is performed successively on subsets of, for example 100, of the randomly placed groups. After a certain number of step intervals, such as 1,000 intervals, the results can be examined to eliminate groups converging to the same minimum. This process is repeated until minimization is complete (e.g. RMS gradient of 0.01 kcal/mole/C). Thus the resulting energy minimized set of molecules comprises what amounts to a set of disconnected fragments in three dimensions representing potential pharmacophores.

The next step then is to connect the pharmacophoric pieces with spacers assembled from small chemical entities (atoms, chains, or ring moieties). In a preferred embodiment, each of the disconnected can be linked in space to generate a single molecule using such computer programs as, for example, NEWLEAD (Tschinke et al. (1993) J Med Chem 36: 3863,3870). The procedure adopted by NEWLEAD executes the following sequence of commands (1) connect two isolated moieties, (2) retain the intermediate solutions for further processing, (3) repeat the above steps for each of the intermediate solutions until no disconnected units are found, and (4) output the final solutions, each of which is single molecule. Such a program can use for example, three types of spacers: library spacers, single-atom spacers, and fuse-ring spacers. The library spacers are optimized structures of small molecules such as ethylene, benzene and methylamide. The output produced by programs such as NEWLEAD consist of a set of molecules containing the original fragments now connected by spacers. The atoms belonging to the input fragments maintain their original orientations in space. The molecules are chemically plausible because of the simple makeup of the spacers and functional groups, and energetically acceptable because of the rejection of solutions with van-der Waals radii violations.

A screening method of the present invention may comprise the following steps:
(i) generating a computer model of a binding pocket using a crystal according to the invention;
(ii) docking a computer representation of a test compound with the computer model;
(iii) analysing the fit of the compound in the binding pocket.

In an aspect of the invention, a method is provided comprising the following steps:
(a) docking a computer representation of a structure of a test compound into a computer representation of a binding pocket of a polo domain in accordance with the invention using a computer program, or by interactively moving the representation of the test compound into the representation of the binding pocket;
(b) characterizing the geometry and the complementary interactions formed between the atoms of the binding pocket and the compound; optionally
(c) searching libraries for molecular fragments which can fit into the empty space between the compound and the binding pocket and can be linked to the compound; and
(d) linking the fragments found in (c) to the compound and evaluating the new modified compound.

In an embodiment of the invention, a method is provided which comprises the following steps:
(a) docking a computer representation of a test compound from a computer data base with a computer representation of a selected binding pocket on a polo domain defined in accordance with the invention to define a complex;
(b) determining a conformation of the complex with a favorable fit and favourable complementary interactions; and
(c) identifying test compounds that best fit the selected binding pocket as potential modulators of the polo domain.

The method may be applied to a plurality of test compounds, to identify those that best fit the selected site.

The model used in the screening method may comprise a binding pocket either alone or in association with one or more ligands and/or cofactors. For example, the model may comprise the binding pocket in association with a nucleotide (or analogue thereof), a substrate (or analogue thereof), and/or modulator.

If the model comprises an unassociated binding pocket, then the selected site under investigation may be the binding pocket itself. The test compound may, for example, mimic a known ligand (e.g. substrate) for a polo family kinase in order to interact with the binding pocket. The selected site may alternatively be another site on the polo domain or polo family kinase.

If the model comprises an associated binding pocket, for example a binding pocket in association with a ligand, the selected site may be the binding pocket or a site made up of the binding pocket and the complexed ligand, or a site on the ligand itself. The test compound may be investigated for its capacity to modulate the interaction with the associated molecule.

A test compound (or plurality of test compounds) may be selected on the basis of their similarity to a known ligand for a polo domain, in particular a Sak or Plk1 polo domain. For example, the screening method may comprise the following steps:
(i) generating a computer model of a binding pocket in complex with a ligand;
(ii) searching for a test compound with a similar three dimensional structure and/or similar chemical groups; and
(iii) evaluating the fit of the test compound in the binding pocket.

Searching may be carried out using a database of computer representations of potential compounds, using methods known in the art.

The present invention also provides a method for designing a ligand for a polo domain. It is well known in the art to use a screening method as described above to identify a test compound with promising fit, but then to use this test compound as a starting point to design a ligand with improved fit to the model. Such techniques are known as “structure-based ligand design” (See Kuntz et al., 1994, Acc. Chem. Res. 27:117; Guida, 1994, Current Opinion in Struc. Biol. 4: 777; and Colman, 1994, Current Opinion in Struc. Biol. 4: 868, for reviews of structure-based drug design and identification;and Kuntz et al 1982, J. Mol. Biol. 162:269; Kuntz et al., 1994, Acc. Chem. Res. 27: 117; Meng et al., 1992, J. Compt. Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG (Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Fuction, and Genetics 8:195);,and, GRID (Goodford, 1985, J. Med. Chem. 28:849).

The method may comprise the following steps:
(i) docking a model of a test compound with a model of a binding pocket;
(ii) identifying one or more groups on the test compound which may be modified to improve their fit in the binding pocket;
(iii) replacing one or more identified groups to produce a modified test compound model; and
(iv) docking the modified test compound model with the model of the binding pocket.

Evaluation of fit may comprise the following steps:
(a) mapping chemical features of a test compound such as by hydrogen bond donors or acceptors, hydrophobic/lipophilic sites, positively ionizable sites, or negatively ionizable sites; and
(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the binding pocket. Preferably the group modifications involve the addition, removal, or replacement of substituents onto the test compound such that the substituents are positioned to collide or to bind preferentially with one or more amino acid residues that correspond to the key amino acid residues of the binding pocket.

If a modified test compound model has an improved fit, then it may bind to a binding pocket and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

The test compound may also be modified “in situ” (i.e. once docked into the potential binding pocket), enabling immediate evaluation of the effect of replacing selected groups. The computer representation of the test compound may be modified by deleting a chemical group or groups, or by adding a chemical group or groups. After each modification to a compound, the atoms of the modified compound and potential binding pocket can be shifted in conformation and the distance between the modulator and the binding pocket atoms may be scored on the basis of geometric fit and favourable complementary interactions between the molecules. This technique is described in detail in Molecular Simulations User Manual, 1995 II LUDI.

Examples of ligand building and/or searching computer include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known ligand for a polo domain. For example, in order to identify potential modulators of a polo domain or polo family kinase, in particular Sak or Plk, a logical approach would be to start with a known ligand to produce a molecule which mimics the binding of the ligand. Such a molecule may, for example, act as a competitive inhibitor for the true ligand, or may bind so strongly that the interaction (and inhibition) is effectively irreversible.

Such a method may comprise the following steps:
(i) generating a computer model of a binding pocket in complex with a ligand;
(ii) replacing one or more groups on the ligand model to produce a modified ligand; and
(iii) evaluating the fit of the modified ligand in the binding pocket.

The replacement groups could be selected and replaced using a compound construction program which replaces computer representations of chemical groups with groups from a computer database, where the representations of the compounds are defined by structural coordinates.

In an embodiment, a screening method is provided for identifying a ligand of a polo domain, in particular a Sak or Plk polo domain, comprising the step of using the structural coordinates of a substrate or component thereof, defined in relation to its spatial association with a binding pocket of the invention, to generate a compound that is capable of associating with the binding pocket.

Screening methods of the present invention may be used to identify compounds or entities that associate with a molecule that associates with a polo domain, in particular a Sak or Plk polo domain.

Test compounds and ligands which are identified using a crystal or model of the present invention can be screened in assays such as those well known in the art. Screening may be for example in vitro, in cell culture, and/or in vivo. Biological screening assays preferably centre on activity-based response models, binding assays (which measure how well a compound binds to a domain), and bacterial, yeast, and animal cell lines (which measure the biological effect of a compound in a cell). The assays may be automated for high throughput screening in which large numbers of compounds can be tested to identify compounds with the desired activity. The biological assay may also be an assay for the binding activity of a compound that selectively binds to the binding pocket compared to other proteins.

Ligands/Compounds Identified by Screening Methods

The present invention provides a ligand or compound identified by a screening method of the present invention. A ligand or compound may have been designed rationally by using a model according to the present invention. A ligand or compound identified using the screening methods of the invention specifically associate with a target compound, or part thereof (e.g. a binding pocket). In the present invention the target compound may be the polo family kinase (e.g. Sak or Plk1) or part thereof (polo domain), or a molecule that is capable of associating with the polo family kinase or polo domain (e.g. substrate).

A ligand or compound identified using a screening method of the invention may act as a “modulator”, i.e. a compound which affects the activity of a polo family kinase, in particular Sak or Plk1. A modulator may reduce, enhance or alter the biological function of a polo family kinase in particular Sak or Plk1. For example a modulator may modulate the capacity of the enzyme to phosphorylate. An alteration in biological function may be characterised by a change in specificity. In order to exert its function, the modulator commonly binds to a binding pocket.

A “modulator” which is capable of reducing the biological function of the enzyme may also be known as an inhibitor. Preferably an inhibitor reduces or blocks the capacity of the enzyme to phosphorylate. The inhibitor may mimic the binding of a substrate, for example, it may be a substrate analogue. A substrate analogue may be designed by considering the interactions between the substrate and a polo domain (for example by using information derivable from the crystal of the invention) and specifically altering one or more groups (as described above).

The present invention also provides a method for modulating the activity of a polo family kinase, in particular Sak or Plk1, using a modulator according to the present invention. It would be possible to monitor activity following such treatment by a number of methods known in the art.

A modulator may be an agonist, partial agonist, partial inverse agonist or antagonist of a polo family kinase.

As used herein, the term “agonist” means any ligand, which is capable of binding to a binding pocket and which is capable of increasing a proportion of the protein that is in an active form, resulting in an increased biological response. The term includes partial agonists and inverse agonists.

As used herein, the term “partial agonist” means an agonist that is unable to evoke the maximal response of a biological system, even at a concentration sufficient to saturate the specific proteins.

As used herein, the term “partial inverse agonist” is an inverse agonist that evokes a submaximal response to a biological system, even at a concentration sufficient to saturate the specific proteins. At high concentrations, it will diminish the actions of a full inverse agonist.

As used herein, the term “antagonist” means any agent that reduces the action of another agent, such as an agonist. The antagonist may act at the same site as the agonist (competitive antagonism). The antagonistic action may result from a combination of the substance being antagonised (chemical antagonism) or the production of an opposite effect through a different protein (functional antagonism or physiological antagonism) or as a consequence of competition for the binding site of an intermediate that links enzyme activation to the effect observed (indirect antagonism).

As used herein, the term “competitive antagonism” refers to the competition between an agonist and an antagonist for a protein that occurs when the binding of agonist and antagonist becomes mutually exclusive. This may be because the agonist and antagonist compete for the same binding sites or combine with adjacent but overlapping sites. A third possibility is that different sites are involved but that they influence the protein macromolecules in such a way that agonist and antagonist molecules cannot be bound at the same time. If the agonist and antagonist form only short lived combinations with the protein so that equilibrium between agonist, antagonist and protein is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare proteins remain.

As mentioned above, an identified ligand or compound may act as a ligand model (for example, a template) for the development of other compounds. A modulator may be a mimetic of a ligand.

Like the test compound (see above) a modulator may be one or a variety of different sorts of molecule.(See examples herein.) A modulator may be an endogenous physiological compound, or it may be a natural or synthetic compound. The modulators of the present invention may be natural or synthetic. The term “modulator” also refers to a chemically modified ligand or compound.

The technique suitable for preparing a modulator will depend on its chemical nature. For example, peptides can be synthesized by solid phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Once cleaved from the resin, the peptide may be purified by preparative high performance liquid chromatography (e.g., Creighton (1983) Proteins Structures and Molecular Principles, WH Freeman and Co, New York N.Y.). The composition of the synthetic peptides may be confirmed by amino acid analysis or sequencing (e.g., the Edman degradation procedure; Creighton, supra).

Organic compounds may be prepared by organic synthetic methods described in references such as March, 1994, Advanced Organic Chemistry: Reactions, Mechanisms, and Structure, New York, McGraw Hill.

The invention also relates to classes of modulators of polo family kinases based on the structure and shape of a substrate or component thereof, defined in relation to the substrate's spatial association with a crystal structure of the invention or part thereof.

The invention contemplates all optical isomers and racemic forms of the modulators of the invention.

Compositions

The present invention also provides the use of a modulator according to the invention, in the manufacture of a medicament to treat and/or prevent a disease in a mammalian patient. There is also provided a pharmaceutical composition comprising such a modulator and a method of treating and/or preventing a disease comprising the step of administering such a modulator or composition to a mammalian patient.

The pharmaceutical compositions may be for human or animal usage in human and veterinary medicine and will typically comprise a pharmaceutically acceptable carrier, diluent, excipient, adjuvant or combination thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may also comprise suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, gel, hydrogel, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example, intravenously, intramuscularly or subcutaneously. For parenteral administration, the compositions may be best used in the form of a sterile aqueous solution which may contain other substances, for example enough salts or monosaccharides to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For buccal or sublingual administration the compositions may be administered in the form of tablets or lozenges which can be formulated in a conventional manner.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

As indicated, the therapeutic agent (e.g. modulator) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Therapeutic administration of polypeptide modulators may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses.

The invention further provides a method of treating a mammal, the method comprising administering to a mammal a modulator or pharmaceutical composition of the present invention.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. By way of example, the pharmaceutical composition of the present invention may be administered in accordance with a regimen of 1 to 10 times per day, such as once or twice per day.

For oral and parenteral administration to human patients, the daily dosage level of the agent may be in single or divided doses.

Applications

The modulators and compositions of the invention may be useful in treating, inhibiting, or preventing diseases modulated by polo family kinases. They may be used to treat, inhibit, or prevent proliferative diseases. The modulators may be used to stimulate or inhibit cell proliferation.

Accordingly, modulators of the invention may be useful in the prevention and treatment of conditions including but not limited to lymphoproliferative conditions, malignant and pre-malignant conditions, arthritis, inflammation, and autoimmune disorders. Malignant and pre-malignant conditions may include solid tumors, B cell lymphomas, chronic lymphocytic leukemia, chronic myelogenous leukemia, prostate hypertrophy, Hirschsprung disease, glioblastoma, breast and ovarian cancer, adenocarcinoma of the salivary gland, premyelocytic leukemia, prostate cancer, multiple endocrine neoplasia type IIA and IIB, medullary thyroid carcinoma, papillary carcinoma, papillary renal carcinoma, hepatocellular carcinoma, gastrointestinal stromal tumors, sporadic mastocytosis, acute myeloid leukemia, large cell lymphoma or Alk lymphoma, chronic myeloid leukemia, hematological/solid tumors, papillary thyroid carcinoma, stem cell leukemia/lymphoma syndrome, acure myelogenous leukemia, osteosarcoma, multiple myeloma, preneoplastic liver foci, and resistance to chemotherapy. Diseases associated with increased cell survival, or the inhibition of apoptosis, include cancers (e.g. follicular lymphomas, carcinomas with p53 mutations, hormone-dependent tumors such as breast cancer, prostate cancer, Kaposi's sarcoma and ovarian cancer); autoimmune disorders (such as lupus erythematosus and immune-related glomerulonephritis rheumatoid arthritis) and viral infections (such as herpes viruses, pox viruses, and adenoviruses); inflammation, graft vs. host disease, acute graft rejection and chronic graft rejection.

Modulators that stimulate cell proliferation may be useful in the treatment of conditions involving damaged cells including conditions in which degeneration of tissue occurs such as arthropathy, bone resorption, inflammatory disease, degenerative disorders of the central nervous system, and for promoting wound healing.

The invention will now be illustrated by the following non-limiting examples:

### EXAMPLES

### Example 1

The following methods were used in the investigation described in the example: Protein expression, mutagenesis and purification: The polo domain of Sak (residues 839 to 925) which was delimited by proteolysis and mass spectrometry, was expressed in E. coli as a GST-fusion protein using the pGEX-2T vector (Pharmacia). The QuikChange™ kit (Stratagene) was used to generate the double site-directed mutant C909L/V874M to improve long-term protein stability and for phasing purposes. Protein was purified by affinity chromatography using glutathione-sepharose (Pharmacia). Bound protein was eluted by cleavage with thrombin (Sigma). Eluate was applied to a HiQ ion-exchange column under low salt conditions. The flow-through containing the polo domain was concentrated to approximately 1 mM and then applied to a Superdex 75 gel filtration column (Pharmacia) for final purification and characterization by static light scattering as described by Luo et al.[35].

Crystallization and data collection: Hanging drops containing 1 μl of 50 mg ml⁻¹ native or mutant protein in 20 mM Hepes pH 8.0, 5 mM dithiothreitol (DTT), were mixed with equal volumes of reservoir buffer containing 100 mM Tris pH 7.0, 32.5% (v/v) Jeffamine M-600 (Hampton), and 200 mM MgCl₂. Hexagonal-like crystals of approximate dimensions 0.10×0.10×0.03 mm were obtained overnight for both native and mutant proteins. The asymmetric unit of the crystals consist of two polypeptides forming an interdigitated dimer. The crystals belong to the space group P3₂12, (a=b 32 51.782 Å, c=146.941 Å).

MAD diffraction data was collected on frozen crystals at the Structural Biology Center 19-BM and BIOCARS 14-BMC at the Advanced Photon Source at Argonne National Laboratory. Data processing and reduction was carried out using HKL 2000 [36]. Heavy atom sites were identified using CNS [37] and phasing, density modification, and experimental electron density map calculation was performed using SHARP³ [38].

Model building and Refinement: Model building was performed using O [39]. A starting model comprised of approximately 85% of the polypeptide sequence was refined using CNS [37]. Bulk solvent correction was applied during refinement and simulated annealing protocols were employed. The remaining structure was built into 2|Fₒ-F_{c}| electron density maps generated with CNS. The final refinement statistics are shown in Table 1. The first and last 6 residues of the polo domain fragment are disordered (residues 839 to 844 and residues 920 to 925) and have not been modeled. Analysis by PROCHECK [40] indicated that no amino acid residues occupy disallowed regions of the Ramachandran plot and 94% occupy the most favored regions.

Sak protein localization: Full length Sak (residues 1-925), Sak_{Δpb} (residues 1-823), Sak₂₄₁ (residues 596-836), Sak_{Δ(pb+241)} (residues 1-595), and Sak_{pb} (residues 824-925) were fused to enhanced green fluorescent protein (EGFP) in the vector pEGFP-Cl (Clontech). NIH 3T3 murine fibroblast cells were maintained in DMEM containing 10% FBS. For transient gene expression, cells at 20-30% confluence on glass cover slips were transiently transfected with pEGFP-Sak, pEGFP-Sak_{Δpb}, pEGFP-Sak_{Δ(pb+241)}, Sak₂₄₁, pEGFP-Sak_{pb}, or pEGFP-Cl with Effectene™ (Qiagen). Cells were released from 48 h of serum starvation by addition of fresh media containing 10% FBS and fixed at intervals as they proceeded through the cell cycle. Cells were processed by rinsing twice in PBS, fixed with 3.7% para-formaldehyde in PBS for 12 min, and permeabilized for 5 min in PBS 0.5% Triton X-100. Actin microfilaments were stained with a 1:100 dilution of TRITC-phalloidin (Sigma) in PBS. γ-tubulil was stained with a 1:200 dilution of anti-γ-tubulin antibody (Sigma) in Tris/Saline 0.1% Tween20 at 20° C. for 40 min. Cells were washed three times in Tris/Saline+0.1% Tween20 and incubated in a 1:500 dilution of rhodamine-conjugated goat anti-mouse antibody (Pierce) for 40 min. Nuclei were stained with Hoechst 33258 (Molecular Probes) in PBS for 1 min. Images were obtained using an Olympus IX-70 inverted microscope equipped with a Princeton CCD camera and Deltavision Deconvolution microscopy software (Applied Precision).

Quantification of EGFP fusion proteins exhibiting centrosomal localization was performed by counting three independent populations of 100 cells. Because of the inability to generate large populations of cells undergoing cytokinesis, the quantification of EGFP fusion protein localization to the cleavage furrow was not scored. The Sak_{Δpd} construct (residues 1-823) fused to EGFP differed from the FLAG- and Myc-tagged Sak_{Δpd} construct (residues 1-836) prepared for coimmunprecipitation studies by a deletion of 13 amino acid residues from the C-terminus. The Sak_{pd} construct (residues 824-925) fused to EGFP differs from the FLAG- and Myc-tagged Sak_{pd} (residues 819-925) prepared for coimmunoprecipitation studies by the deletion of 5 amino acid residues at the N-terminus.

Immmunoprecipitation: NIH 3T3 murine fibroblast cells were maintained in DMEM containing 10% FBS. For transient gene expression, cells at 30-40% confluence were tranlsfected using Effectene™ (Qiagen). After 24 h post transfection cells were lysed in 50 mM Tris pH 7.5, 100 mM NaCl, 1 mM EDTA, 0.5% Triton-X 100. Immunoprecipitations were performed using anti-FLAG antibody (Sigma) and Protein G Sepharose (Pharmacia) according to product specifications. The Protein G sepharose matrix was washed three times with lysis buffer. Western blots were performed using a 1:200 dilution of anti-Myc antibody (Santa Cruz Biotech) or a 1:4000 dilution of anti-FLAG antibody (Sigma).

Coordinates

The Sak polo domain coordinates are in Table 2.

Results and Discussison

A protein fragment encompassing the polo box motif of Sak (residues 839 to 925) was expressed and characterized. Using limited proteolysis and mass spectrometry, it was found that the polo box motif comprises an autonomously folding unit, which is designated the polo domain, that behaves as a dimer in solution as indicated by size exclusion chromatography and static light scattering analysis (SLS molecular weight=22.6±0.9 kDa versus predicted monomer molecular weight=10.8 kDa). The domain was crystallized and its structure determined using the selenomethione-multiple anomalous dispersion (SeMet MAD) method. Structure determination and crystallographic refinement statistics are provided in Table 1. A comprehensive structure based sequence alignment of the polo domain is shown in FIG. 1. Ribbons and molecular surface representations of the polo domain structure and a stereo view of representative electron density of the MAD experimental map are shown in FIG. 2.

Structure Description

The crystal structure of the polo domain of Sak is dimeric, consisting of two α-helices and two six-stranded β-sheets (FIG. 2A, FIG. 2B). Analysis by VAST [18] identifies this structure as a novel protein fold. The topology of one polypeptide subunit of the dimer consists of, from its N- to C-terminus, an extended strand segment (Ex1), five β-strands (β1-β5) one α-helix (αA)₁ and a C-terminal β-strand (β6). β-strands 6, 1, 2, and 3 from one subunit form a contiguous anti parallel β-sheet with β-strands 4 and 5 from the second subunit. The two \overline{β}-sheets pack with a crossing angle of 110°, orienting the hydrophobic surfaces inward and the hydrophilic surfaces outward. Helix αA, which is colinear with β strand 6 of the same polypeptide, buries a large portion of the non-overlapping hydrophobic β-sheet surfaces. Interactions involving helices αA comprise a majority of the hydrophobic core structure and also the dimer interface. The total surface area buried by dimer formation is 2448 Å². Overall, the dimeric structure is clam like (60 Å×44 Å×22 Å), hinged at one end through the seamless association of β-strand 3 from each subunit (FIG. 2B). Extending inwards from the mouth of the structure is a deep cavity of approximate dimensions 17 Å×8 Å×12 Å (FIG. 2A, FIG. 2B). The entry to this cavity is divided in two by the contact of the Trp 853 side chains on β-strand 1 from each polypeptide of the dimer. Strands Ex1 from each polypeptide designate the proximal ends of the cleft (FIG. 2B).

Residues of Sak that compose much of the polo domain hydrophobic core are highly conserved across the Plks (FIG. 1). Mutation of one hydrophobic core position, Leu 427 to Ala in Plk1 (equivalent to Leu 857 in Sak), disrupts the ability of Plk1 to complement the cdc5-1 temperature-sensitive mitotic arrest phenotype in yeast [13]. This mutation may disrupt the overall polo domain fold. A large proportion of the conserved hydrophobic core residues (13 out of 19) also participate in dimer formation. Only two charged residues, equivalent to Asp 868 and Lys 906 in Sak, are conserved among most polo domains and these residues participate in dimerization through a 2.6 Å intermolecular salt bridge in the crystal structure (FIG. 2A, FIG. 2B). Together, these observations indicate that the dimeric fold revealed by the crystal structure may be a functionally relevant conformation accessible by all polo domains.

The presence of two polo domains in all Plks other than the Sak orthologs raises an interesting possibility for an intramolecular mode of polo domain dimerization. In support of this possibility is a covariance in primary structure across paired polo domains involving the conserved salt bridge (Asp 868 and Lys 906) and a dimer interface residue equivalent to Val 846 in Sak (FIG. 2A, FIG. 2B). Val 846, which lies in close proximity to the conserved salt bridge, is substituted with aspartic acid in the first, but not the second, polo domain of the Plks. This hydrophobic-to-charged amino acid substitution appears to be compensated by the substitution of Lys 906 with Arg (K906R) in the second polo domain. Modeling studies suggest that this concerted substitution would allow for the formation of a bidentate salt interaction between the arginine and two aspartic acid residues, facilitated by the increased hydrogen bonding capacity of the arginine guanidinium group (FIG. 2A, inset). In further support of the possibility for an intramolecular mode of dimerization, the linker region between tandem polo domains is sufficiently long (21 to 37 amino acids) in all Plks to bridge the 36 Å distance separating the amino and carboxy termini of opposing dimer chains in the polo domain crystal structure.

While less conserved than the hydrophobic core and dimer interface structure, the interfacial cleft and pocket display properties suggestive of a functionally important surface. Of the 19 conserved hydrophobic positions in the polo domain alignment, 9 contribute side chains to the outer cleft and inner pocket (FIG. 1). Modeling of the polo domain sequences of Fnk/Prk, Snk, and Plk1 to form an intramolecular dimer, shows that the approximate dimensions and hydrophobic character of the pocket and cleft region are also generally preserved. Polo domain mutations in Plk1 and Cdc5 that disrupt localization or the ability to complement the cdc5-1 temperature sensitive mutation in yeast map mostly to the interfacial cleft region [13, 15]. These include the mutations W414F and V415A in Plk1 or W517F and V518A in Cdc5 (equivalent to Lys 844 and Ser 845 in Sak) which locate within or just precede strand Ex I at the proximal ends of the cleft. Indeed, the cdc5-1 temperature-sensitive mutation itself (P511L) maps to the region proceeding strand Ex1 and a third mutation in Plk1, N437D (equivalent to Asn 867 in the β2-β3 linker of Sak), is positioned to influence the conformation of strand Ex1. In the Sak polo domain structure, Asn 867 forms intramolecular hydrogen bonds with backbone amino and carbonyl groups of the Ex1 strand residues Phe 847 and Ser 845. These observations suggest that the interfacial cleft and pocket region is functionally important, possibly composing a ligand-binding site.

Polo Domain Self-Association in vivo

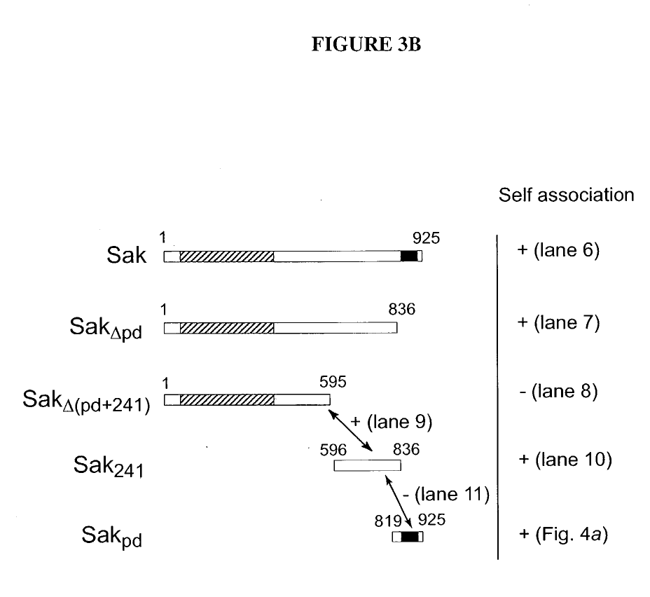

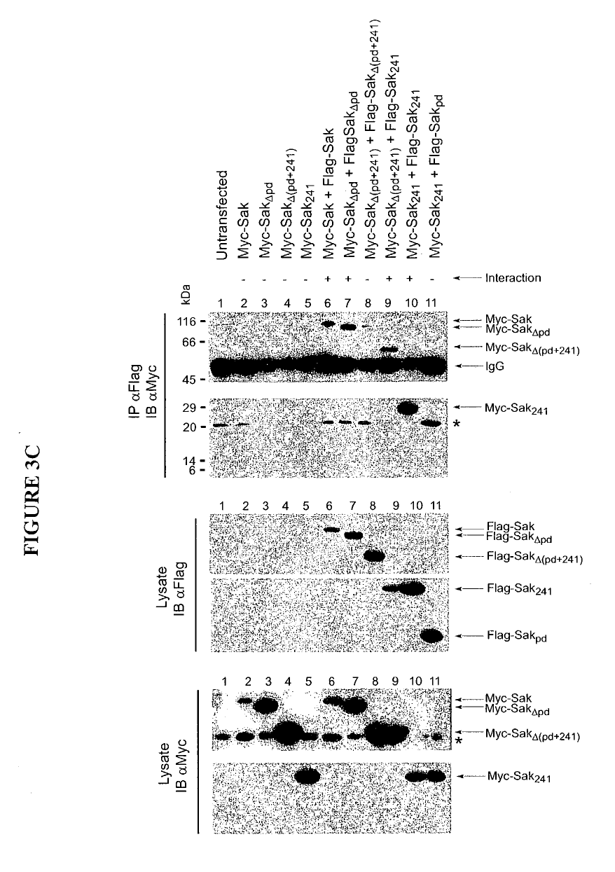

To investigate the ability of the polo domain of Sak to dimerize in vivo differentially tagged mammalian expression constructs were generated and tested for sell-association in vivo using a coimmunoprecipitation assay. As shown in FIG. 3A, the Myc-tagged polo domain of Sak (Sak_{pd}) was coimmunoprecipitated with a FLAG-tagged polo domain when both constructs were transfected into NIH 3T3 cells. This confirms the potential of the isolated domain to self-associate in vivo. To determine whether full-length Sak can self-associate and whether self-association is polo domain-dependent, immunoprecipitations were performed with similarly tagged expression constructs (FIG. 3B). As shown in FIG. 3C, immunoprecipitation of FLAG-tagged, full-length Sak yielded Myc-tagged Sak, confirming the self-association of full-length Sak in vivo (lane 6). However, deletion of the polo domain (Sak_{Δpd}) did not abolish this association (lane 7) while a more extensive C-terminal deletion, Sak_{Δ(pd+241)}, (lane 8) did. Further analysis revealed that the 241 amino acid region N-terminal to the polo domain, Sak₂₄₁, was sufficient for self-association (lane 10) and was also able to associate with regions N-terminal (lane 9) but not C-terminal (lane 11) to itself. A BLAST [19] analysis of the primary structure of Sak₂₄₁ reveals high sequence conservation amongst Sak orthologs but not other Plk family members, and analysis with SMART [20] and PROSITE [21] reveals no similarity to known motifs or domains involved in protein-protein interaction. Together these data suggest that the polo domain of Sak can self-associate in vivo but regions N-terminal to the polo domain can also mediate the self-association of the full-length molecule.

Polo Domain Subcellular Localization

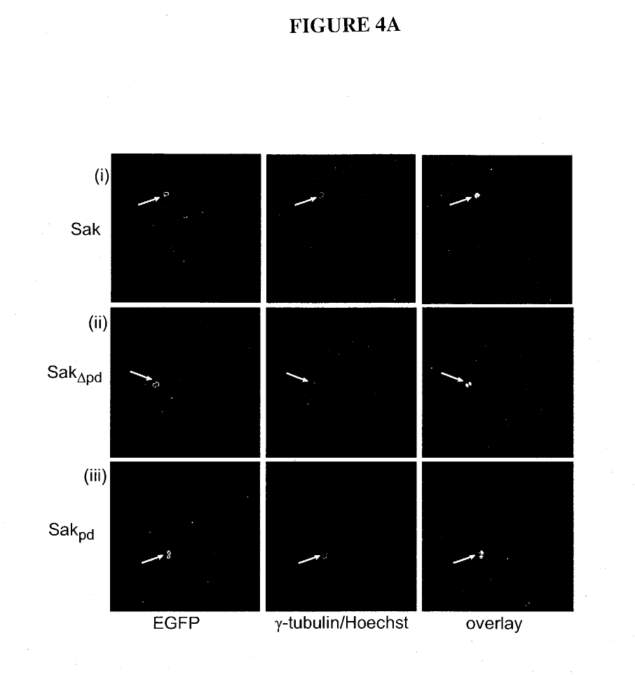

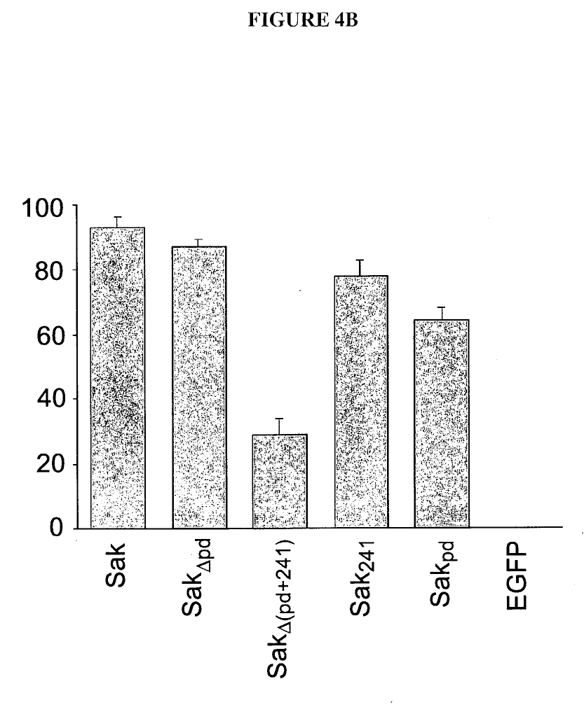

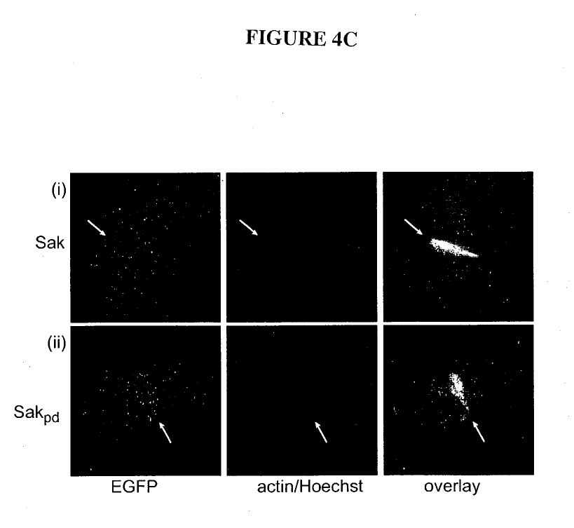

To investigate the role of the polo domain in the subcellular localization of Sak, enhanced green fluorescent protein (EGFP) fusion constructs of Sak, Sak_{Δpd}, Sak_{Δ(pd+241)}, Sak₂₄₁, and Sak_{pd} were transiently transfected into NIH 3T3 cells and examined using immunofluorescence. EGFP-Sak colocalizes in cells with γ-tubulin and actin, which indicate the positions of centrosomes and the cleavage furrow, respectively (FIG. 4A, panel i; FIG. 4C, panel i). Localization to these structures has been demonstrated for full-length Plk 1, Cdc5, and Sak [9, 13, 15]. The experiments show that the isolated polo domain of Sak localizes to centrosomes and the cleavage furrow (FIG. 4A, panel iii; FIG. 4C, panel ii), which is consistent with previous observations for larger C-terminal protein fragments encompassing the polo domains of Cdc5 and Plk1 [15, 22]. Unexpectedly, deletion of the polo domain (Sak_{Δpd}) did not abolish the subcellular localization of Sak (FIG. 4A, panel ii), although the larger of two C-terminal deletions, Sak_{Δ(pd+241)}, did reduce the efficiency of localization to centrosomes from 93% to 24% lo in comparison to full length Sak (FIG. 4B). Sak₂₄, also localizes efficiently to centrosomes demonstrating that residues 596 to 836 of Sak are also sufficient for subcellular localization (FIG. 4B). These observations conflict with the results of mutational studies of Plk1 and Cdc5 in yeast in which the polo domains appear to be essential for localization [13, 15]. This discrepancy may reflect the presence of a second localization domain unique to Sak or alternatively may reflect the ability of regions outside of the polo domain to promote an association with endogenous Sak in NIH 3T3 cells.

### SUMMARY

The polo domain of Sak forms dimers both in vitro and in a crystal environment, can self-associate in vivo, and localizes to mitotic structures. The conservation of the hydrophobic core and dimer interface residues, the presence of two copies of the polo domain in most Plks, and the covariance across tandem polo domains in most Plks suggest that the ability to adopt a dimeric conformation may be a general characteristic of all polo domains and that dimerization may occur in an intramolecular manner for some family members.

The deregulation of Plks alters mitotic checkpoints, chromosome stability and can lead to tumour development [27, 28]. Indeed, Plk1 is overexpressed in many human tumours [29-32] and causes malignant transformation when overexpressed in NIH 3T3 cells [33]. In addition, over expression of a kinase-deficient form of Plk1 results in cell death, an apparent dominant-negative effect that is more pronounced in tumor cells than non-transformed cells [34]. This identifies the Plks as potential targets for cancer therapy. The requirement of the polo domain for Plk family function and, in contrast to the catalytic domain, its exclusive presence in this small family of proteins that regulate mitotic progression suggests that the polo domain itself may serve as a good target for intervention. Indeed, the large semi-enclosed cleft and pocket with its partial hydrophobic character appears well suited for the design of small molecule inhibitors.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Data collection and refinement statistics

| | | | | | | | | Phasing |
|---|---|---|---|---|---|---|---|---|
| | | Resolution | Reflections | | Completeness¹ | | R-sym^{1.2} | Power³ |
| | λ(Å) | (Å) | Total/Unique | Redundancy | (%) | I/σ¹ | (%) | (iso/ano) |
| Inflection | 0.9790 | 2.00 | 131753/15710 | 8.4 | 99.9(99.5) | 32.0(4.0) | 6.2(33.1) | 1.63/2.68 |
| Peak | 0.9788 | 2.00 | 135549/15708 | 8.6 | 100.0(100.0) | 34.5(5.0) | 6.2(29.6) | −/2.84 |
| Remote1 | 0.9640 | 2.00 | 118899/15704 | 7.6 | 98.7(90.5) | 23.7(2.1) | 6.7(45.5) | 1.43/2.04 |
| Remote2 | 0.9940 | 2.00 | 122313/15672 | 7.8 | 99.8(98.8) | 29.6(3.4) | 5.6(37.2) | 1.10/0.54 |

| | |
|---|---|
| Refinement Statistics: | |
| Resolution (Å) | 50-2 |
| Reflections: | |
| All data | 15,511 |
| \|F\| > 2 σ | 14,153 |
| R-factor/R_{free} (%)⁴ | |
| All data | 22.65/24.75 |
| \|F\| > 2 σ | 21.85/24.16 |
| Average B value (Å²) | 28 |
| R.m.s deviation | |
| Bond angles (°) | 1.51 |
| Bond lengths (Å) | 0.012 |
| B-factor for main chain bonds (Å²) | 1.65 |
| Number of Atoms | |
| Non-hydrogen protein | 1,168 |
| Water molecules | 58 |

TABLE 2
REMARK coordinates from minimization and B-factor refinement
”
REMARK refinement resolution: 500.0-2.0 A
”
REMARK starting r = 0.2341 free_r = 0.2471
”
REMARK final r = 0.2312 free_r = 0.2489
”
REMARK rmsd bonds = 0.011680 rmsd angles = 1.53873
REMARK B rmsd for bonded mainchain atoms = 1.546 target = 1.5
REMARK B rmsd for bonded sidechain atoms = 2.297 target = 2.0
REMARK B rmsd for angle mainchain atoms = 2.242 target = 2.0
REMARK B rmsd for angle sidechain atoms = 3.450 target = 2.5
REMARK target = mlf final wa = 2.67
REMARK final rweight = 0.1829 (with wa = 2.67)
REMARK md-method = torsion annealing schedule = slowcool
REMARK starting temperature = 600 total md steps = 6 * 6
REMARK cycles = 2 coordinate steps = 20 B-factor steps = 10
REMARK sg = P3(2)12 a = 51.782 b = 51.782 c = 146.941 alpha = 90 beta = 90 gamma = 120
REMARK topology file 1: CNS_TOPPAR: protein.top
REMARK topology file 2: CNS_TOPPAR: dna-rna.top
REMARK topology file 3: CNS_TOPPAR: water.top
REMARK topology file 4: CNS_TOPPAR: ion.top
REMARK parameter file 1: CNS_TOPPAR: protein_rep.pararn
REMARK parameter file 2: CNS_TOPPAR: dna-rna_rep.param
REMARK parameter file 3: CNS_TOPPAR: water_rep.param
REMARK parameter file 4: CNS_TOPPAR: ion.param
REMARK molecular structure file: automatic
REMARK input coordinates: refine28.pdb
REMARK reflection file = peak1.cv
REMARK ncs = none
REMARK B-correction resolution: 6.0-2.0
REMARK initial B-factor correction applied to fobs:
REMARK B11 = −1.018 B22 = −1.018 B33 = 2.036
REMARK B12 = −3.420 B13 = 0.000 B23 = 0.000
REMARK B-factor correction applied to coordinate array B: −1.378
REMARK bulk solvent: density level = 0.389731 e/A\hat{ }3, B-factor = 59.3227 A\hat{ }2
REMARK reflections with |Fobs|/sigma_F < 0.0 rejected
REMARK reflections with |Fobs| > 10000 * rms(Fobs) rejected
REMARK anomalous diffraction data was input

| | | |
|---|---|---|
| REMARK theoretical total number of refl. in resol. range: | 29811 | (100.0%) |
| REMARK number of unobserved reflections (no entry or \|F\| = 0): | 537 | (1.8%) |
| REMARK number of reflections rejected: | 0 | (0.0%) |
| REMARK total number of reflections used: | 29274 | (98.2%) |
| REMARK number of reflections in working set: | 26435 | (88.7%) |
| REMARK number of reflections in test set: | 2839 | (9.5%) |

CRYST1 51.782 51.782 146.941 90.00 90.00 120.00 P 32 1 2
REMARK FILENAME =“refine29.pdb″
REMARK DATE: 11-Jan-01 15:10:17 created by user: leung
REMARK VERSION: 1.0

| | | | | | | | | | | | |
|---|---|---|---|---|---|---|---|---|---|---|---|
| ATOM | 1 | CB | SER | A | 8 | 18.661 | 18.360 | 26.264 | 1.00 | 48.49 | A |
| ATOM | 2 | OG | SER | A | 8 | 19.163 | 19.370 | 27.127 | 1.00 | 50.47 | A |
| ATOM | 3 | C | SER | A | 8 | 16.981 | 16.981 | 27.538 | 1.00 | 45.01 | A |
| ATOM | 4 | O | SER | A | 8 | 16.148 | 16.296 | 26.940 | 1.00 | 45.75 | A |
| ATOM | 5 | N | SER | A | 8 | 18.698 | 15.879 | 26.153 | 1.00 | 47.55 | A |
| ATOM | 6 | CA | SER | A | 8 | 18.430 | 17.054 | 27.040 | 1.00 | 47.07 | A |
| ATOM | 7 | N | VAL | A | 9 | 16.678 | 17.677 | 28.629 | 1.00 | 41.83 | A |
| ATOM | 8 | CA | VAL | A | 9 | 15.323 | 17.661 | 29.172 | 1.00 | 38.86 | A |
| ATOM | 9 | CB | VAL | A | 9 | 15.355 | 17.713 | 30.720 | 1.00 | 39.15 | A |
| ATOM | 10 | CG1 | VAL | A | 9 | 16.094 | 18.970 | 31.181 | 1.00 | 40.82 | A |
| ATOM | 11 | CG2 | VAL | A | 9 | 13.937 | 17.705 | 31.280 | 1.00 | 39.80 | A |
| ATOM | 12 | C | VAL | A | 9 | 14.511 | 18.853 | 28.641 | 1.00 | 36.67 | A |
| ATOM | 13 | O | VAL | A | 9 | 15.001 | 19.985 | 28.591 | 1.00 | 35.40 | A |
| ATOM | 14 | N | PHE | A | 10 | 13.280 | 18.603 | 28.216 | 1.00 | 33.75 | A |
| ATOM | 15 | CA | PHE | A | 10 | 12.457 | 19.698 | 27.740 | 1.00 | 33.36 | A |
| ATOM | 16 | CB | PHE | A | 10 | 12.733 | 19.956 | 26.242 | 1.00 | 36.21 | A |
| ATOM | 17 | CG | PHE | A | 10 | 12.536 | 18.753 | 25.366 | 1.00 | 38.49 | A |
| ATOM | 18 | CD1 | PHE | A | 10 | 11.284 | 18.460 | 24.834 | 1.00 | 39.36 | A |
| ATOM | 19 | CD2 | PHE | A | 10 | 13.590 | 17.888 | 25.105 | 1.00 | 40.33 | A |
| ATOM | 20 | CE1 | PHE | A | 10 | 11.083 | 17.326 | 24.059 | 1.00 | 38.97 | A |
| ATOM | 21 | CE2 | PHE | A | 10 | 13.393 | 16.740 | 24.323 | 1.00 | 40.13 | A |
| ATOM | 22 | CZ | PHE | A | 10 | 12.137 | 16.461 | 23.802 | 1.00 | 39.15 | A |
| ATOM | 23 | C | PHE | A | 10 | 10.992 | 19.393 | 28.002 | 1.00 | 31.66 | A |
| ATOM | 24 | O | PHE | A | 10 | 10.615 | 18.234 | 28.224 | 1.00 | 29.02 | A |
| ATOM | 25 | N | VAL | A | 11 | 10.170 | 20.442 | 28.016 | 1.00 | 28.88 | A |
| ATOM | 26 | CA | VAL | A | 11 | 8.731 | 20.291 | 28.239 | 1.00 | 28.95 | A |
| ATOM | 27 | CB | VAL | A | 11 | 8.050 | 21.686 | 28.417 | 1.00 | 29.73 | A |
| ATOM | 28 | CG1 | VAL | A | 11 | 6.538 | 21.516 | 28.643 | 1.00 | 29.60 | A |
| ATOM | 29 | CG2 | VAL | A | 11 | 8.678 | 22.417 | 29.601 | 1.00 | 27.93 | A |
| ATOM | 30 | C | VAL | A | 11 | 8.109 | 19.572 | 27.041 | 1.00 | 29.58 | A |
| ATOM | 31 | O | VAL | A | 11 | 8.447 | 19.861 | 25.896 | 1.00 | 31.80 | A |
| ATOM | 32 | N | LYS | A | 12 | 7.215 | 18.633 | 27.295 | 1.00 | 28.97 | A |
| ATOM | 33 | CA | LYS | A | 12 | 6.568 | 17.898 | 26.223 | 1.00 | 29.28 | A |
| ATOM | 34 | CB | LYS | A | 12 | 6.699 | 16.405 | 26.485 | 1.00 | 31.10 | A |
| ATOM | 35 | CG | LYS | A | 12 | 5.974 | 15.520 | 25.517 | 1.00 | 34.59 | A |
| ATOM | 36 | CD | LYS | A | 12 | 6.087 | 14.074 | 25.981 | 1.00 | 38.87 | A |
| ATOM | 37 | CE | LYS | A | 12 | 5.482 | 13.130 | 24.951 | 1.00 | 42.52 | A |
| ATOM | 38 | NZ | LYS | A | 12 | 5.795 | 11.708 | 25.274 | 1.00 | 45.41 | A |
| ATOM | 39 | C | LYS | A | 12 | 5.076 | 18.286 | 26.153 | 1.00 | 28.89 | A |
| ATOM | 40 | O | LYS | A | 12 | 4.551 | 18.561 | 25.062 | 1.00 | 28.53 | A |
| ATOM | 41 | N | ASN | A | 13 | 4.406 | 18.293 | 27.310 | 1.00 | 26.75 | A |
| ATOM | 42 | CA | ASN | A | 13 | 2.983 | 18.663 | 27.398 | 1.00 | 25.69 | A |
| ATOM | 43 | CB | ASN | A | 13 | 2.066 | 17.447 | 27.594 | 1.00 | 25.72 | A |
| ATOM | 44 | CG | ASN | A | 13 | 2.329 | 16.336 | 26.595 | 1.00 | 28.05 | A |
| ATOM | 45 | OD1 | ASN | A | 13 | 2.591 | 16.596 | 25.416 | 1.00 | 28.45 | A |
| ATOM | 46 | ND2 | ASN | A | 13 | 2.234 | 15.088 | 27.053 | 1.00 | 26.70 | A |
| ATOM | 47 | C | ASN | A | 13 | 2.798 | 19.568 | 28.603 | 1.00 | 26.66 | A |
| ATOM | 48 | O | ASN | A | 13 | 3.458 | 19.381 | 29.640 | 1.00 | 24.38 | A |
| ATOM | 49 | N | VAL | A | 14 | 1.891 | 20.544 | 28.471 | 1.00 | 27.40 | A |
| ATOM | 50 | CA | VAL | A | 14 | 1.570 | 21.488 | 29.547 | 1.00 | 27.04 | A |
| ATOM | 51 | CB | VAL | A | 14 | 2.240 | 22.862 | 29.355 | 1.00 | 29.56 | A |
| ATOM | 52 | CG1 | VAL | A | 14 | 1.802 | 23.808 | 30.463 | 1.00 | 30.90 | A |
| ATOM | 53 | CG2 | VAL | A | 14 | 3.722 | 22.735 | 29.412 | 1.00 | 29.82 | A |
| ATOM | 54 | C | VAL | A | 14 | 0.064 | 21.728 | 29.556 | 1.00 | 27.95 | A |
| ATOM | 55 | O | VAL | A | 14 | −0.604 | 21.699 | 28.505 | 1.00 | 25.07 | A |
| ATOM | 56 | N | GLY | A | 15 | −0.476 | 21.964 | 30.743 | 1.00 | 27.09 | A |
| ATOM | 57 | CA | GLY | A | 15 | −1.895 | 22.227 | 30.851 | 1.00 | 25.98 | A |
| ATOM | 58 | C | GLY | A | 15 | −2.156 | 23.066 | 32.083 | 1.00 | 25.20 | A |
| ATOM | 59 | O | GLY | A | 15 | −1.290 | 23.206 | 32.957 | 1.00 | 23.65 | A |
| ATOM | 60 | N | TRP | A | 16 | −3.333 | 23.666 | 32.150 | 1.00 | 23.21 | A |
| ATOM | 61 | CA | TRP | A | 16 | −3.667 | 24.451 | 33.319 | 1.00 | 22.53 | A |
| ATOM | 62 | CB | TRP | A | 16 | −3.016 | 25.848 | 33.284 | 1.00 | 22.44 | A |
| ATOM | 63 | CG | TRP | A | 16 | −3.597 | 26.857 | 32.302 | 1.00 | 26.17 | A |
| ATOM | 64 | CD2 | TRP | A | 16 | −2.857 | 27.662 | 31.373 | 1.00 | 27.23 | A |
| ATOM | 65 | CE2 | TRP | A | 16 | −3.782 | 28.549 | 30.753 | 1.00 | 28.23 | A |
| ATOM | 66 | CE3 | TRP | A | 16 | −1.507 | 27.720 | 31.000 | 1.00 | 29.82 | A |
| ATOM | 67 | CD1 | TRP | A | 16 | −4.908 | 27.275 | 32.206 | 1.00 | 25.56 | A |
| ATOM | 68 | NE1 | TRP | A | 16 | −5.020 | 28.298 | 31.278 | 1.00 | 26.09 | A |
| ATOM | 69 | CZ2 | TRP | A | 16 | −3.380 | 29.490 | 29.788 | 1.00 | 30.09 | A |
| ATOM | 70 | CZ3 | TRP | A | 16 | −1.112 | 28.666 | 30.033 | 1.00 | 30.90 | A |
| ATOM | 71 | CH2 | TRP | A | 16 | −2.047 | 29.531 | 29.442 | 1.00 | 29.35 | A |
| ATOM | 72 | C | TRP | A | 16 | −5.153 | 24.578 | 33.418 | 1.00 | 21.37 | A |
| ATOM | 73 | O | TRP | A | 16 | −5.898 | 24.354 | 32.437 | 1.00 | 19.90 | A |
| ATOM | 74 | N | ALA | A | 17 | −5.607 | 24.930 | 34.614 | 1.00 | 21.50 | A |
| ATOM | 75 | CA | ALA | A | 17 | −7.029 | 25.121 | 34.810 | 1.00 | 21.42 | A |
| ATOM | 76 | CB | ALA | A | 17 | −7.662 | 23.858 | 35.340 | 1.00 | 19.91 | A |
| ATOM | 77 | C | ALA | A | 17 | −7.040 | 26.193 | 35.850 | 1.00 | 22.81 | A |
| ATOM | 78 | O | ALA | A | 17 | −6.495 | 25.978 | 36.936 | 1.00 | 21.78 | A |
| ATOM | 79 | N | THR | A | 18 | −7.623 | 27.349 | 35.519 | 1.00 | 21.72 | A |
| ATOM | 80 | CA | THR | A | 18 | −7.675 | 28.458 | 36.462 | 1.00 | 24.80 | A |
| ATOM | 81 | CB | THR | A | 18 | −6.944 | 29.715 | 35.917 | 1.00 | 26.48 | A |
| ATOM | 82 | OG1 | THR | A | 18 | −7.603 | 30.182 | 34.731 | 1.00 | 25.58 | A |
| ATOM | 83 | CG2 | THR | A | 18 | −5.474 | 29.377 | 35.563 | 1.00 | 27.63 | A |
| ATOM | 84 | C | THR | A | 18 | −9.110 | 28.850 | 36.813 | 1.00 | 26.36 | A |
| ATOM | 85 | O | THR | A | 18 | −10.050 | 28.600 | 36.054 | 1.00 | 25.24 | A |
| ATOM | 86 | N | GLN | A | 19 | −9.265 | 29.438 | 37.990 | 1.00 | 28.51 | A |
| ATOM | 87 | CA | GLN | A | 19 | −10.561 | 29.886 | 38.473 | 1.00 | 31.86 | A |
| ATOM | 88 | CB | GLN | A | 19 | −10.869 | 29.229 | 39.804 | 1.00 | 33.51 | A |
| ATOM | 89 | CG | GLN | A | 19 | −10.742 | 27.730 | 39.762 | 1.00 | 37.86 | A |
| ATOM | 90 | CD | GLN | A | 19 | −11.609 | 27.085 | 40.817 | 1.00 | 43.09 | A |
| ATOM | 91 | OE1 | GLN | A | 19 | −12.846 | 27.247 | 40.802 | 1.00 | 44.57 | A |
| ATOM | 92 | NE2 | GLN | A | 19 | −10.978 | 26.359 | 41.757 | 1.00 | 43.84 | A |
| ATOM | 93 | C | GLN | A | 19 | −10.471 | 31.399 | 38.634 | 1.00 | 33.17 | A |
| ATOM | 94 | O | GLN | A | 19 | −10.072 | 32.083 | 37.695 | 1.00 | 37.10 | A |
| ATOM | 95 | N | LEU | A | 20 | −10.821 | 31.949 | 39.790 | 1.00 | 31.93 | A |
| ATOM | 96 | CA | LEU | A | 20 | −10.729 | 33.414 | 39.928 | 1.00 | 30.19 | A |
| ATOM | 97 | CB | LEU | A | 20 | −11.811 | 33.972 | 40.864 | 1.00 | 31.42 | A |
| ATOM | 98 | CG | LEU | A | 20 | −13.246 | 34.105 | 40.339 | 1.00 | 35.07 | A |
| ATOM | 99 | CD1 | LEU | A | 20 | −13.979 | 35.172 | 41.179 | 1.00 | 34.87 | A |
| ATOM | 100 | CD2 | LEU | A | 20 | −13.226 | 34.554 | 38.891 | 1.00 | 34.43 | A |
| ATOM | 101 | C | LEU | A | 20 | −9.383 | 33.893 | 40.438 | 1.00 | 27.07 | A |
| ATOM | 102 | O | LEU | A | 20 | −8.738 | 34.721 | 39.814 | 1.00 | 27.58 | A |
| ATOM | 103 | N | THR | A | 21 | −8.964 | 33.383 | 41.585 | 1.00 | 24.20 | A |
| ATOM | 104 | CA | THR | A | 21 | −7.679 | 33.818 | 42.154 | 1.00 | 23.31 | A |
| ATOM | 105 | CB | THR | A | 21 | −7.886 | 34.596 | 43.477 | 1.00 | 22.88 | A |
| ATOM | 106 | OG1 | THR | A | 21 | −8.645 | 33.787 | 44.374 | 1.00 | 23.64 | A |
| ATOM | 107 | CG2 | THR | A | 21 | −8.683 | 35.898 | 43.232 | 1.00 | 22.54 | A |
| ATOM | 108 | C | THR | A | 21 | −6.736 | 32.644 | 42.442 | 1.00 | 23.22 | A |
| ATOM | 109 | O | THR | A | 21 | −5.842 | 32.757 | 43.281 | 1.00 | 23.48 | A |
| ATOM | 110 | N | SER | A | 22 | −6.948 | 31.512 | 41.783 | 1.00 | 22.33 | A |
| ATOM | 111 | CA | SER | A | 22 | −6.069 | 30.359 | 42.011 | 1.00 | 22.54 | A |
| ATOM | 112 | CB | SER | A | 22 | −6.518 | 29.553 | 43.237 | 1.00 | 22.65 | A |
| ATOM | 113 | OG | SER | A | 22 | −7.758 | 28.909 | 42.998 | 1.00 | 24.68 | A |
| ATOM | 114 | C | SER | A | 22 | −6.119 | 29.484 | 40.773 | 1.00 | 22.86 | A |
| ATOM | 115 | O | SER | A | 22 | −6.958 | 29.678 | 39.881 | 1.00 | 21.28 | A |
| ATOM | 116 | N | GLY | A | 23 | −5.198 | 28.533 | 40.689 | 1.00 | 21.88 | A |
| ATOM | 117 | CA | GLY | A | 23 | −5.218 | 27.670 | 39.530 | 1.00 | 21.62 | A |
| ATOM | 118 | C | GLY | A | 23 | −4.258 | 26.524 | 39.733 | 1.00 | 21.90 | A |
| ATOM | 119 | O | GLY | A | 23 | −3.533 | 26.484 | 40.734 | 1.00 | 20.10 | A |
| ATOM | 120 | N | ALA | A | 24 | −4.248 | 25.609 | 38.770 | 1.00 | 22.43 | A |
| ATOM | 121 | CA | ALA | A | 24 | −3.386 | 24.450 | 38.832 | 1.00 | 21.38 | A |
| ATOM | 122 | CB | ALA | A | 24 | −4.225 | 23.200 | 39.129 | 1.00 | 21.35 | A |
| ATOM | 123 | C | ALA | A | 24 | −2.702 | 24.353 | 37.483 | 1.00 | 23.49 | A |
| ATOM | 124 | O | ALA | A | 24 | −3.282 | 24.702 | 36.430 | 1.00 | 22.42 | A |
| ATOM | 125 | N | VAL | A | 25 | −1.438 | 23.939 | 37.508 | 1.00 | 21.72 | A |
| ATOM | 126 | CA | VAL | A | 25 | −0.674 | 23.808 | 36.281 | 1.00 | 23.76 | A |
| ATOM | 127 | CB | VAL | A | 25 | 0.529 | 24.789 | 36.233 | 1.00 | 26.05 | A |
| ATOM | 128 | CG1 | VAL | A | 25 | 1.391 | 24.500 | 34.978 | 1.00 | 26.10 | A |
| ATOM | 129 | CG2 | VAL | A | 25 | 0.038 | 26.224 | 36.209 | 1.00 | 31.11 | A |
| ATOM | 130 | C | VAL | A | 25 | −0.110 | 22.413 | 36.238 | 1.00 | 24.23 | A |
| ATOM | 131 | O | VAL | A | 25 | 0.331 | 21.897 | 37.274 | 1.00 | 23.93 | A |
| ATOM | 132 | N | TRP | A | 26 | −0.115 | 21.798 | 35.063 | 1.00 | 24.25 | A |
| ATOM | 133 | CA | TRP | A | 26 | 0.458 | 20.459 | 34.916 | 1.00 | 24.49 | A |
| ATOM | 134 | CB | TRP | A | 26 | −0.590 | 19.439 | 34.495 | 1.00 | 28.72 | A |
| ATOM | 135 | CG | TRP | A | 26 | −0.006 | 18.132 | 33.934 | 1.00 | 32.20 | A |
| ATOM | 136 | CD2 | TRP | A | 26 | −0.077 | 17.668 | 32.567 | 1.00 | 33.87 | A |
| ATOM | 137 | CE2 | TRP | A | 26 | 0.516 | 16.374 | 32.524 | 1.00 | 35.35 | A |
| ATOM | 138 | CE3 | TRP | A | 26 | −0.592 | 18.220 | 31.371 | 1.00 | 34.27 | A |
| ATOM | 139 | CD1 | TRP | A | 26 | 0.622 | 17.133 | 34.642 | 1.00 | 33.50 | A |
| ATOM | 140 | NE1 | TRP | A | 26 | 0.935 | 16.070 | 33.801 | 1.00 | 35.95 | A |
| ATOM | 141 | CZ2 | TRP | A | 26 | 0.605 | 15.621 | 31.342 | 1.00 | 35.87 | A |
| ATOM | 142 | CZ3 | TRP | A | 26 | −0.504 | 17.472 | 30.191 | 1.00 | 34.20 | A |
| ATOM | 143 | CH2 | TRP | A | 26 | 0.090 | 16.182 | 30.189 | 1.00 | 35.90 | A |
| ATOM | 144 | C | TRP | A | 26 | 1.509 | 20.527 | 33.829 | 1.00 | 25.41 | A |
| ATOM | 145 | O | TRP | A | 26 | 1.363 | 21.274 | 32.828 | 1.00 | 23.08 | A |
| ATOM | 146 | N | VAL | A | 27 | 2.575 | 19.746 | 34.000 | 1.00 | 22.86 | A |
| ATOM | 147 | CA | VAL | A | 27 | 3.626 | 19.745 | 33.002 | 1.00 | 23.49 | A |
| ATOM | 148 | CB | VAL | A | 27 | 4.733 | 20.742 | 33.342 | 1.00 | 25.20 | A |
| ATOM | 149 | CG1 | VAL | A | 27 | 5.803 | 20.704 | 32.237 | 1.00 | 25.19 | A |
| ATOM | 150 | CG2 | VAL | A | 27 | 4.136 | 22.176 | 33.430 | 1.00 | 25.58 | A |
| ATOM | 151 | C | VAL | A | 27 | 4.221 | 18.361 | 32.915 | 1.00 | 24.58 | A |
| ATOM | 152 | O | VAL | A | 27 | 4.435 | 17.713 | 33.943 | 1.00 | 21.32 | A |
| ATOM | 153 | N | GLN | A | 28 | 4.421 | 17.891 | 31.688 | 1.00 | 23.14 | A |
| ATOM | 154 | CA | GLN | A | 28 | 5.029 | 16.590 | 31.490 | 1.00 | 27.34 | A |
| ATOM | 155 | CB | GLN | A | 28 | 4.051 | 15.642 | 30.812 | 1.00 | 30.52 | A |
| ATOM | 156 | CG | GLN | A | 28 | 4.662 | 14.304 | 30.539 | 1.00 | 37.65 | A |
| ATOM | 157 | CD | GLN | A | 28 | 3.611 | 13.255 | 30.231 | 1.00 | 41.99 | A |
| ATOM | 158 | OE1 | GLN | A | 28 | 2.730 | 13.465 | 29.378 | 1.00 | 43.60 | A |
| ATOM | 159 | NE2 | GLN | A | 28 | 3.696 | 12.110 | 30.924 | 1.00 | 42.95 | A |
| ATOM | 160 | C | GLN | A | 28 | 6.282 | 16.778 | 30.640 | 1.00 | 26.40 | A |
| ATOM | 161 | O | GLN | A | 28 | 6.239 | 17.410 | 29.576 | 1.00 | 23.98 | A |
| ATOM | 162 | N | PHE | A | 29 | 7.403 | 16.247 | 31.122 | 1.00 | 24.75 | A |
| ATOM | 163 | CA | PHE | A | 29 | 8.658 | 16.395 | 30.423 | 1.00 | 24.70 | A |
| ATOM | 164 | CB | PHE | A | 29 | 9.781 | 16.608 | 31.423 | 1.00 | 26.58 | A |
| ATOM | 165 | CG | PHE | A | 29 | 9.580 | 17.805 | 32.295 | 1.00 | 25.63 | A |
| ATOM | 166 | CD1 | PHE | A | 29 | 9.006 | 17.669 | 33.563 | 1.00 | 26.22 | A |
| ATOM | 167 | CD2 | PHE | A | 29 | 9.966 | 19.071 | 31.851 | 1.00 | 25.52 | A |
| ATOM | 168 | CE1 | PHE | A | 29 | 8.815 | 18.782 | 34.380 | 1.00 | 24.43 | A |
| ATOM | 169 | CE2 | PHE | A | 29 | 9.786 | 20.184 | 32.645 | 1.00 | 25.07 | A |
| ATOM | 170 | CZ | PHE | A | 29 | 9.207 | 20.045 | 33.923 | 1.00 | 26.84 | A |
| ATOM | 171 | C | PHE | A | 29 | 8.996 | 15.236 | 29.506 | 1.00 | 25.07 | A |
| ATOM | 172 | O | PHE | A | 29 | 8.348 | 14.185 | 29.559 | 1.00 | 24.61 | A |
| ATOM | 173 | N | ASN | A | 30 | 10.027 | 15.400 | 28.685 | 1.00 | 25.40 | A |
| ATOM | 174 | CA | ASN | A | 30 | 10.347 | 14.329 | 27.742 | 1.00 | 28.37 | A |
| ATOM | 175 | CB | ASN | A | 30 | 11.397 | 14.786 | 26.727 | 1.00 | 28.60 | A |
| ATOM | 176 | CG | ASN | A | 30 | 12.721 | 15.053 | 27.363 | 1.00 | 33.92 | A |
| ATOM | 177 | OD1 | ASN | A | 30 | 12.813 | 15.827 | 28.320 | 1.00 | 34.59 | A |
| ATOM | 178 | ND2 | ASN | A | 30 | 13.780 | 14.407 | 26.844 | 1.00 | 36.62 | A |
| ATOM | 179 | C | ASN | A | 30 | 10.812 | 13.048 | 28.410 | 1.00 | 27.80 | A |
| ATOM | 180 | O | ASN | A | 30 | 10.751 | 11.990 | 27.790 | 1.00 | 28.37 | A |
| ATOM | 181 | N | ASP | A | 31 | 11.267 | 13.134 | 29.662 | 1.00 | 26.69 | A |
| ATOM | 182 | CA | ASP | A | 31 | 11.741 | 11.951 | 30.371 | 1.00 | 27.04 | A |
| ATOM | 183 | CB | ASP | A | 31 | 12.777 | 12.327 | 31.432 | 1.00 | 27.21 | A |
| ATOM | 184 | CG | ASP | A | 31 | 12.207 | 13.219 | 32.528 | 1.00 | 27.15 | A |
| ATOM | 185 | OD1 | ASP | A | 31 | 11.000 | 13.506 | 32.534 | 1.00 | 25.45 | A |
| ATOM | 186 | OD2 | ASP | A | 31 | 12.979 | 13.628 | 33.401 | 1.00 | 27.89 | A |
| ATOM | 187 | C | ASP | A | 31 | 10.612 | 11.178 | 31.020 | 1.00 | 27.64 | A |
| ATOM | 188 | O | ASP | A | 31 | 10.855 | 10.187 | 31.700 | 1.00 | 25.26 | A |
| ATOM | 189 | N | GLY | A | 32 | 9.375 | 11.613 | 30.786 | 1.00 | 26.83 | A |
| ATOM | 190 | CA | GLY | A | 32 | 8.242 | 10.921 | 31.376 | 1.00 | 25.75 | A |
| ATOM | 191 | C | GLY | A | 32 | 7.840 | 11.475 | 32.734 | 1.00 | 25.58 | A |
| ATOM | 192 | O | GLY | A | 32 | 6.804 | 11.089 | 33.273 | 1.00 | 26.85 | A |
| ATOM | 193 | N | SER | A | 33 | 8.631 | 12.373 | 33.307 | 1.00 | 25.39 | A |
| ATOM | 194 | CA | SER | A | 33 | 8.271 | 12.878 | 34.632 | 1.00 | 24.37 | A |
| ATOM | 195 | CB | SER | A | 33 | 9.485 | 13.468 | 35.356 | 1.00 | 23.52 | A |
| ATOM | 196 | OG | SER | A | 33 | 10.048 | 14.588 | 34.676 | 1.00 | 21.76 | A |
| ATOM | 197 | C | SER | A | 33 | 7.177 | 13.923 | 34.501 | 1.00 | 24.25 | A |
| ATOM | 198 | O | SER | A | 33 | 6.883 | 14.385 | 33.386 | 1.00 | 22.02 | A |
| ATOM | 199 | N | GLN | A | 34 | 6.565 | 14.264 | 35.628 | 1.00 | 22.89 | A |
| ATOM | 200 | CA | GLN | A | 34 | 5.475 | 15.245 | 35.648 | 1.00 | 24.56 | A |
| ATOM | 201 | CB | GLN | A | 34 | 4.111 | 14.551 | 35.584 | 1.00 | 25.19 | A |
| ATOM | 202 | CG | GLN | A | 34 | 3.920 | 13.489 | 34.537 | 1.00 | 30.40 | A |
| ATOM | 203 | CD | GLN | A | 34 | 2.618 | 12.744 | 34.764 | 1.00 | 33.54 | A |
| ATOM | 204 | OE1 | GLN | A | 34 | 1.532 | 13.323 | 34.629 | 1.00 | 32.79 | A |
| ATOM | 205 | NE2 | GLN | A | 34 | 2.713 | 11.464 | 35.143 | 1.00 | 32.46 | A |
| ATOM | 206 | C | GLN | A | 34 | 5.455 | 16.073 | 36.927 | 1.00 | 24.21 | A |
| ATOM | 207 | O | GLN | A | 34 | 5.776 | 15.580 | 38.015 | 1.00 | 23.24 | A |
| ATOM | 208 | N | LEU | A | 35 | 5.025 | 17.324 | 36.783 | 1.00 | 22.82 | A |
| ATOM | 209 | CA | LEU | A | 35 | 4.867 | 18.239 | 37.906 | 1.00 | 21.35 | A |
| ATOM | 210 | CB | LEU | A | 35 | 5.722 | 19.501 | 37.728 | 1.00 | 20.77 | A |
| ATOM | 211 | CG | LEU | A | 35 | 7.243 | 19.483 | 37.911 | 1.00 | 19.81 | A |
| ATOM | 212 | CD1 | LEU | A | 35 | 7.865 | 20.779 | 37.381 | 1.00 | 19.63 | A |
| ATOM | 213 | CD2 | LEU | A | 35 | 7.529 | 19.316 | 39.402 | 1.00 | 19.87 | A |
| ATOM | 214 | C | LEU | A | 35 | 3.393 | 18.676 | 37.867 | 1.00 | 22.42 | A |
| ATOM | 215 | O | LEU | A | 35 | 2.844 | 18.900 | 36.780 | 1.00 | 19.09 | A |
| ATOM | 216 | N | VAL | A | 36 | 2.752 | 18.744 | 39.030 | 1.00 | 21.77 | A |
| ATOM | 217 | CA | VAL | A | 36 | 1.376 | 19.250 | 39.131 | 1.00 | 23.89 | A |
| ATOM | 218 | CB | VAL | A | 36 | 0.344 | 18.161 | 39.558 | 1.00 | 26.38 | A |
| ATOM | 219 | CG1 | VAL | A | 36 | −1.021 | 18.822 | 39.858 | 1.00 | 25.49 | A |
| ATOM | 220 | CG2 | VAL | A | 36 | 0.152 | 17.146 | 38.434 | 1.00 | 22.63 | A |
| ATOM | 221 | C | VAL | A | 36 | 1.553 | 20.285 | 40.229 | 1.00 | 26.68 | A |
| ATOM | 222 | O | VAL | A | 36 | 2.053 | 19.964 | 41.324 | 1.00 | 25.04 | A |
| ATOM | 223 | N | MET | A | 37 | 1.178 | 21.532 | 39.933 | 1.00 | 25.65 | A |
| ATOM | 224 | CA | MET | A | 37 | 1.352 | 22.615 | 40.878 | 1.00 | 26.26 | A |
| ATOM | 225 | CB | MET | A | 37 | 2.440 | 23.554 | 40.356 | 1.00 | 25.44 | A |
| ATOM | 226 | CG | MET | A | 37 | 3.613 | 22.779 | 39.756 | 1.00 | 30.14 | A |
| ATOM | 227 | SD | MET | A | 37 | 4.975 | 23.772 | 39.196 | 1.00 | 32.21 | A |
| ATOM | 228 | CE | MET | A | 37 | 4.108 | 24.902 | 38.187 | 1.00 | 30.46 | A |
| ATOM | 229 | C | MET | A | 37 | 0.080 | 23.398 | 41.090 | 1.00 | 25.73 | A |
| ATOM | 230 | O | MET | A | 37 | −0.753 | 23.513 | 40.170 | 1.00 | 27.70 | A |
| ATOM | 231 | N | GLN | A | 38 | −0.115 | 23.903 | 42.304 | 1.00 | 23.37 | A |
| ATOM | 232 | CA | GLN | A | 38 | −1.292 | 24.728 | 42.545 | 1.00 | 22.97 | A |
| ATOM | 233 | CB | GLN | A | 38 | −2.157 | 24.137 | 43.644 | 1.00 | 24.17 | A |
| ATOM | 234 | CG | GLN | A | 38 | −2.892 | 22.891 | 43.116 | 1.00 | 26.79 | A |
| ATOM | 235 | CD | GLN | A | 38 | −3.932 | 22.394 | 44.054 | 1.00 | 28.97 | A |
| ATOM | 236 | OE1 | GLN | A | 38 | −4.754 | 23.164 | 44.537 | 1.00 | 31.62 | A |
| ATOM | 237 | NE2 | GLN | A | 38 | −3.930 | 21.093 | 44.314 | 1.00 | 31.46 | A |
| ATOM | 238 | C | GLN | A | 38 | −0.711 | 26.095 | 42.890 | 1.00 | 22.53 | A |
| ATOM | 239 | O | GLN | A | 38 | 0.348 | 26.187 | 43.530 | 1.00 | 21.65 | A |
| ATOM | 240 | N | ALA | A | 39 | −1.365 | 27.153 | 42.410 | 1.00 | 21.23 | A |
| ATOM | 241 | CA | ALA | A | 39 | −0.882 | 28.517 | 42.615 | 1.00 | 20.50 | A |
| ATOM | 242 | CB | ALA | A | 39 | −0.262 | 29.028 | 41.305 | 1.00 | 20.80 | A |
| ATOM | 243 | C | ALA | A | 39 | −2.023 | 29.450 | 43.056 | 1.00 | 21.59 | A |
| ATOM | 244 | O | ALA | A | 39 | −3.178 | 29.095 | 42.927 | 1.00 | 19.34 | A |
| ATOM | 245 | N | GLY | A | 40 | −1.692 | 30.645 | 43.542 | 1.00 | 21.15 | A |
| ATOM | 246 | CA | GLY | A | 40 | −2.733 | 31.570 | 43.994 | 1.00 | 23.40 | A |
| ATOM | 247 | C | GLY | A | 40 | −2.278 | 33.015 | 43.871 | 1.00 | 22.86 | A |
| ATOM | 248 | O | GLY | A | 40 | −1.081 | 33.294 | 43.873 | 1.00 | 22.47 | A |
| ATOM | 249 | N | VAL | A | 41 | −3.230 | 33.939 | 43.764 | 1.00 | 22.89 | A |
| ATOM | 250 | CA | VAL | A | 41 | −2.913 | 35.355 | 43.628 | 1.00 | 22.01 | A |
| ATOM | 251 | CB | VAL | A | 41 | −4.080 | 36.073 | 42.888 | 1.00 | 22.10 | A |
| ATOM | 252 | CG1 | VAL | A | 41 | −3.840 | 37.577 | 42.814 | 1.00 | 22.14 | A |
| ATOM | 253 | CG2 | VAL | A | 41 | −4.228 | 35.478 | 41.469 | 1.00 | 21.24 | A |
| ATOM | 254 | C | VAL | A | 41 | −2.728 | 35.948 | 45.039 | 1.00 | 21.72 | A |
| ATOM | 255 | O | VAL | A | 41 | −3.617 | 35.791 | 45.900 | 1.00 | 21.08 | A |
| ATOM | 256 | N | SER | A | 42 | −1.599 | 36.624 | 45.287 | 1.00 | 18.13 | A |
| ATOM | 257 | CA | SER | A | 42 | −1.355 | 37.238 | 46.608 | 1.00 | 19.79 | A |
| ATOM | 258 | CB | SER | A | 42 | 0.132 | 37.205 | 46.980 | 1.00 | 19.38 | A |
| ATOM | 259 | OG | SER | A | 42 | 0.925 | 37.570 | 45.856 | 1.00 | 18.21 | A |
| ATOM | 260 | C | SER | A | 42 | −1.786 | 38.696 | 46.642 | 1.00 | 21.07 | A |
| ATOM | 261 | O | SER | A | 42 | −1.907 | 39.283 | 47.721 | 1.00 | 20.90 | A |
| ATOM | 262 | N | SER | A | 43 | −1.931 | 39.310 | 45.474 | 1.00 | 20.00 | A |
| ATOM | 263 | CA | SER | A | 43 | −2.395 | 40.705 | 45.442 | 1.00 | 20.47 | A |
| ATOM | 264 | CB | SER | A | 43 | −1.286 | 41.685 | 45.872 | 1.00 | 22.34 | A |
| ATOM | 265 | OG | SER | A | 43 | −0.244 | 41.758 | 44.915 | 1.00 | 27.90 | A |
| ATOM | 266 | C | SER | A | 43 | −2.918 | 41.089 | 44.079 | 1.00 | 20.54 | A |
| ATOM | 267 | O | SER | A | 43 | −2.405 | 40.643 | 43.031 | 1.00 | 18.49 | A |
| ATOM | 268 | N | ILE | A | 44 | −3.965 | 41.907 | 44.099 | 1.00 | 19.49 | A |
| ATOM | 269 | CA | ILE | A | 44 | −4.591 | 42.385 | 42.891 | 1.00 | 20.54 | A |
| ATOM | 270 | CB | ILE | A | 44 | −6.049 | 41.893 | 42.793 | 1.00 | 22.31 | A |
| ATOM | 271 | CG2 | ILE | A | 44 | −6.695 | 42.444 | 41.543 | 1.00 | 19.72 | A |
| ATOM | 272 | CG1 | ILE | A | 44 | −6.084 | 40.351 | 42.790 | 1.00 | 22.18 | A |
| ATOM | 273 | CD1 | ILE | A | 44 | −7.485 | 39.724 | 42.708 | 1.00 | 23.31 | A |
| ATOM | 274 | C | ILE | A | 44 | −4.577 | 43.909 | 42.905 | 1.00 | 22.19 | A |
| ATOM | 275 | O | ILE | A | 44 | −5.088 | 44.546 | 43.843 | 1.00 | 20.82 | A |
| ATOM | 276 | N | SER | A | 45 | −3.969 | 44.487 | 41.881 | 1.00 | 21.03 | A |
| ATOM | 277 | CA | SER | A | 45 | −3.901 | 45.939 | 41.747 | 1.00 | 22.60 | A |
| ATOM | 278 | CB | SER | A | 45 | −2.449 | 46.372 | 41.535 | 1.00 | 25.70 | A |
| ATOM | 279 | OG | SER | A | 45 | −2.321 | 47.782 | 41.474 | 1.00 | 27.90 | A |
| ATOM | 280 | C | SER | A | 45 | −4.756 | 46.262 | 40.531 | 1.00 | 22.78 | A |
| ATOM | 281 | O | SER | A | 45 | −4.403 | 45.928 | 39.377 | 1.00 | 22.24 | A |
| ATOM | 282 | N | TYR | A | 46 | −5.901 | 46.880 | 40.798 | 1.00 | 22.65 | A |
| ATOM | 283 | CA | TYR | A | 46 | −6.862 | 47.225 | 39.763 | 1.00 | 22.55 | A |
| ATOM | 284 | CB | TYR | A | 46 | −8.269 | 46.831 | 40.218 | 1.00 | 22.32 | A |
| ATOM | 285 | CG | TYR | A | 46 | −9.361 | 47.219 | 39.231 | 1.00 | 25.37 | A |
| ATOM | 286 | CD1 | TYR | A | 46 | −9.518 | 46.508 | 38.037 | 1.00 | 24.33 | A |
| ATOM | 287 | CE1 | TYR | A | 46 | −10.515 | 46.850 | 37.108 | 1.00 | 24.37 | A |
| ATOM | 288 | CD2 | TYR | A | 46 | −10.246 | 48.303 | 39.488 | 1.00 | 24.12 | A |
| ATOM | 289 | CE2 | TYR | A | 46 | −11.254 | 48.652 | 38.562 | 1.00 | 23.53 | A |
| ATOM | 29.0 | CZ | TYR | A | 46 | −11.375 | 47.920 | 37.373 | 1.00 | 25.93 | A |
| ATOM | 291 | OH | TYR | A | 46 | −12.325 | 48.233 | 36.425 | 1.00 | 24.28 | A |
| ATOM | 292 | C | TYR | A | 46 | −6.842 | 48.720 | 39.455 | 1.00 | 24.28 | A |
| ATOM | 293 | O | TYR | A | 46 | −6.968 | 49.565 | 40.357 | 1.00 | 23.43 | A |
| ATOM | 294 | N | THR | A | 47 | −6.656 | 49.041 | 38.183 | 1.00 | 23.62 | A |
| ATOM | 295 | CA | THR | A | 47 | −6.675 | 50.421 | 37.739 | 1.00 | 24.08 | A |
| ATOM | 296 | CB | THR | A | 47 | −5.469 | 50.732 | 36.843 | 1.00 | 24.70 | A |
| ATOM | 297 | OG1 | THR | A | 47 | −4.278 | 50.549 | 37.615 | 1.00 | 25.79 | A |
| ATOM | 298 | CG2 | THR | A | 47 | −5.504 | 52.202 | 36.344 | 1.00 | 24.86 | A |
| ATOM | 299 | C | THR | A | 47 | −7.970 | 50.597 | 36.953 | 1.00 | 23.37 | A |
| ATOM | 300 | O | THR | A | 47 | −8.169 | 49.957 | 35.926 | 1.00 | 22.53 | A |
| ATOM | 301 | N | SER | A | 48 | −8.863 | 51.427 | 37.478 | 1.00 | 22.12 | A |
| ATOM | 302 | CA | SER | A | 48 | −10.145 | 51.709 | 36.838 | 1.00 | 20.42 | A |
| ATOM | 303 | CB | SER | A | 48 | −11.014 | 52.577 | 37.771 | 1.00 | 22.04 | A |
| ATOM | 304 | OG | SER | A | 48 | −12.030 | 53.285 | 37.022 | 1.00 | 23.62 | A |
| ATOM | 305 | C | SER | A | 48 | −9.967 | 52.459 | 35.543 | 1.00 | 19.42 | A |
| ATOM | 306 | O | SER | A | 48 | −8.908 | 53.040 | 35.279 | 1.00 | 19.34 | A |
| ATOM | 307 | N | PRO | A | 49 | −11.002 | 52.454 | 34.691 | 1.00 | 19.82 | A |
| ATOM | 308 | CD | PRO | A | 49 | −12.265 | 51.693 | 34.726 | 1.00 | 20.00 | A |
| ATOM | 309 | CA | PRO | A | 49 | −10.871 | 53.193 | 33.442 | 1.00 | 21.05 | A |
| ATOM | 310 | CB | PRO | A | 49 | −12.221 | 52.980 | 32.778 | 1.00 | 21.03 | A |
| ATOM | 311 | CG | PRO | A | 49 | −12.626 | 51.626 | 33.274 | 1.00 | 22.27 | A |
| ATOM | 312 | C | PRO | A | 49 | −10.644 | 54.676 | 33.777 | 1.00 | 23.30 | A |
| ATOM | 313 | O | PRO | A | 49 | −10.110 | 55.416 | 32.958 | 1.00 | 24.51 | A |
| ATOM | 314 | N | ASP | A | 50 | −11.065 | 55.118 | 34.972 | 1.00 | 22.65 | A |
| ATOM | 315 | CA | ASP | A | 50 | −10.882 | 56.531 | 35.338 | 1.00 | 24.70 | A |
| ATOM | 316 | CB | ASP | A | 50 | −11.983 | 57.025 | 36.312 | 1.00 | 21.88 | A |
| ATOM | 317 | CG | ASP | A | 50 | −11.898 | 56.421 | 37.707 | 1.00 | 24.64 | A |
| ATOM | 318 | OD1 | ASP | A | 50 | −10.847 | 55.844 | 38.052 | 1.00 | 22.86 | A |
| ATOM | 319 | OD2 | ASP | A | 50 | −12.899 | 56.547 | 38.485 | 1.00 | 22.31 | A |
| ATOM | 320 | C | ASP | A | 50 | −9.491 | 56.860 | 35.876 | 1.00 | 23.99 | A |
| ATOM | 321 | O | ASP | A | 50 | −9.240 | 57.980 | 36.312 | 1.00 | 23.78 | A |
| ATOM | 322 | N | GLY | A | 51 | −8.579 | 55.887 | 35.833 | 1.00 | 23.93 | A |
| ATOM | 323 | CA | GLY | A | 51 | −7.207 | 56.127 | 36.294 | 1.00 | 22.64 | A |
| ATOM | 324 | C | GLY | A | 51 | −6.904 | 55.899 | 37.762 | 1.00 | 23.25 | A |
| ATOM | 325 | O | GLY | A | 51 | −5.742 | 55.965 | 38.161 | 1.00 | 25.45 | A |
| ATOM | 326 | N | GLN | A | 52 | −7.918 | 55.629 | 38.580 | 1.00 | 22.74 | A |
| ATOM | 327 | CA | GLN | A | 52 | −7.688 | 55.398 | 40.006 | 1.00 | 24.31 | A |
| ATOM | 328 | CB | GLN | A | 52 | −8.971 | 55.644 | 40.805 | 1.00 | 25.55 | A |
| ATOM | 329 | CG | GLN | A | 52 | −9.422 | 57.116 | 40.840 | 1.00 | 27.26 | A |
| ATOM | 330 | CD | GLN | A | 52 | −8.415 | 57.965 | 41.581 | 1.00 | 28.20 | A |
| ATOM | 331 | OE1 | GLN | A | 52 | −7.610 | 58.667 | 40.976 | 1.00 | 28.60 | A |
| ATOM | 332 | NE2 | GLN | A | 52 | −8.439 | 57.879 | 42.905 | 1.00 | 30.60 | A |
| ATOM | 333 | C | GLN | A | 52 | −7.235 | 53.951 | 40.241 | 1.00 | 23.71 | A |
| ATOM | 334 | O | GLN | A | 52 | −7.838 | 53.023 | 39.709 | 1.00 | 22.37 | A |
| ATOM | 335 | N | THR | A | 53 | −6.206 | 53.762 | 41.053 | 1.00 | 23.96 | A |
| ATOM | 336 | CA | THR | A | 53 | −5.730 | 52.404 | 41.328 | 1.00 | 26.22 | A |
| ATOM | 337 | CB | THR | A | 53 | −4.203 | 52.267 | 41.093 | 1.00 | 26.01 | A |
| ATOM | 338 | OG1 | THR | A | 53 | −3.922 | 52.437 | 39.701 | 1.00 | 27.10 | A |
| ATOM | 339 | CG2 | THR | A | 53 | −3.721 | 50.858 | 41.500 | 1.00 | 27.86 | A |
| ATOM | 340 | C | THR | A | 53 | −6.036 | 51.967 | 42.746 | 1.00 | 27.22 | A |
| ATOM | 341 | O | THR | A | 53 | −5.855 | 52.743 | 43.689 | 1.00 | 28.02 | A |
| ATOM | 342 | N | THR | A | 54 | −6.513 | 50.726 | 42.888 | 1.00 | 24.96 | A |
| ATOM | 343 | CA | THR | A | 54 | −6.840 | 50.159 | 44.190 | 1.00 | 25.21 | A |
| ATOM | 344 | CB | THR | A | 54 | −8.368 | 50.007 | 44.390 | 1.00 | 27.26 | A |
| ATOM | 345 | OG1 | THR | A | 54 | −9.035 | 51.243 | 44.070 | 1.00 | 29.29 | A |
| ATOM | 346 | CG2 | THR | A | 54 | −8.658 | 49.649 | 45.830 | 1.00 | 28.65 | A |
| ATOM | 347 | C | THR | A | 54 | −6.200 | 48.771 | 44.304 | 1.00 | 24.23 | A |
| ATOM | 348 | O | THR | A | 54 | −6.286 | 47.951 | 43.381 | 1.00 | 21.76 | A |
| ATOM | 349 | N | ARG | A | 55 | −5.523 | 48.524 | 45.419 | 1.00 | 23.84 | A |
| ATOM | 350 | CA | ARG | A | 55 | −4.881 | 47.236 | 45.634 | 1.00 | 23.81 | A |
| ATOM | 351 | CB | ARG | A | 55 | −3.453 | 47.456 | 46.146 | 1.00 | 24.59 | A |
| ATOM | 352 | CG | ARG | A | 55 | −2.679 | 46.172 | 46.450 | 1.00 | 32.74 | A |
| ATOM | 353 | CD | ARG | A | 55 | −1.368 | 46.412 | 47.241 | 1.00 | 34.85 | A |
| ATOM | 354 | NE | ARG | A | 55 | −0.955 | 45.153 | 47.863 | 1.00 | 42.09 | A |
| ATOM | 355 | CZ | ARG | A | 55 | −1.475 | 44.645 | 48.983 | 1.00 | 42.52 | A |
| ATOM | 356 | NH1 | ARG | A | 55 | −2.429 | 45.293 | 49.649 | 1.00 | 44.55 | A |
| ATOM | 357 | NH2 | ARG | A | 55 | −1.072 | 43.454 | 49.414 | 1.00 | 42.98 | A |
| ATOM | 358 | C | ARG | A | 55 | −5.684 | 46.398 | 46.638 | 1.00 | 25.42 | A |
| ATOM | 359 | O | ARG | A | 55 | −6.163 | 46.906 | 47.676 | 1.00 | 24.29 | A |
| ATOM | 360 | N | TYR | A | 56 | −5.858 | 45.118 | 46.322 | 1.00 | 22.94 | A |
| ATOM | 361 | CA | TYR | A | 56 | −6.556 | 44.210 | 47.227 | 1.00 | 24.10 | A |
| ATOM | 362 | CB | TYR | A | 56 | −7.803 | 43.627 | 46.563 | 1.00 | 24.74 | A |
| ATOM | 363 | CG | TYR | A | 56 | −8.775 | 44.697 | 46.115 | 1.00 | 25.46 | A |
| ATOM | 364 | CD1 | TYR | A | 56 | −8.568 | 45.387 | 44.918 | 1.00 | 25.25 | A |
| ATOM | 365 | CE1 | TYR | A | 56 | −9.432 | 46.391 | 44.504 | 1.00 | 27.98 | A |
| ATOM | 366 | CD2 | TYR | A | 56 | −9.884 | 45.041 | 46.900 | 1.00 | 26.40 | A |
| ATOM | 367 | CE2 | TYR | A | 56 | −10.765 | 46.054 | 46.496 | 1.00 | 28.27 | A |
| ATOM | 368 | CZ | TYR | A | 56 | −10.526 | 46.720 | 45.294 | 1.00 | 29.75 | A |
| ATOM | 369 | OH | TYR | A | 56 | −11.372 | 47.718 | 44.876 | 1.00 | 33.43 | A |
| ATOM | 370 | C | TYR | A | 56 | −5.613 | 43.085 | 47.638 | 1.00 | 23.96 | A |
| ATOM | 371 | O | TYR | A | 56 | −5.070 | 42.367 | 46.789 | 1.00 | 23.65 | A |
| ATOM | 372 | N | GLY | A | 57 | −5.377 | 42.979 | 48.936 | 1.00 | 22.44 | A |
| ATOM | 373 | CA | GLY | A | 57 | −4.519 | 41.939 | 49.450 | 1.00 | 25.69 | A |
| ATOM | 374 | C | GLY | A | 57 | −5.270 | 40.617 | 49.404 | 1.00 | 25.67 | A |
| ATOM | 375 | O | GLY | A | 57 | −6.484 | 40.589 | 49.174 | 1.00 | 23.94 | A |
| ATOM | 376 | N | GLU | A | 58 | −4.558 | 39.523 | 49.659 | 1.00 | 26.09 | A |
| ATOM | 377 | CA | GLU | A | 58 | −5.161 | 38.205 | 49.580 | 1.00 | 26.78 | A |
| ATOM | 378 | CB | GLU | A | 58 | −4.079 | 37.145 | 49.798 | 1.00 | 28.97 | A |
| ATOM | 379 | CG | GLU | A | 58 | −4.507 | 35.750 | 49.403 | 1.00 | 31.47 | A |
| ATOM | 380 | CD | GLU | A | 58 | −3.325 | 34.781 | 49.186 | 1.00 | 34.27 | A |
| ATOM | 381 | OE1 | GLU | A | 58 | −3.614 | 33.595 | 48.943 | 1.00 | 35.62 | A |
| ATOM | 382 | OE2 | GLU | A | 58 | −2.129 | 35.193 | 49.243 | 1.00 | 32.05 | A |
| ATOM | 383 | C | GLU | A | 58 | −6.305 | 38.002 | 50.557 | 1.00 | 27.07 | A |
| ATOM | 384 | O | GLU | A | 58 | −7.247 | 37.245 | 50.272 | 1.00 | 26.31 | A |
| ATOM | 385 | N | ASN | A | 59 | −6.222 | 38.666 | 51.710 | 1.00 | 25.53 | A |
| ATOM | 386 | CA | ASN | A | 59 | −7.260 | 38.535 | 52.726 | 1.00 | 27.19 | A |
| ATOM | 387 | CB | ASN | A | 59 | −6.609 | 38.444 | 54.108 | 1.00 | 26.47 | A |
| ATOM | 388 | CG | ASN | A | 59 | −5.726 | 37.212 | 54.234 | 1.00 | 28.80 | A |
| ATOM | 389 | OD1 | ASN | A | 59 | −5.957 | 36.209 | 53.537 | 1.00 | 28.28 | A |
| ATOM | 390 | ND2 | ASN | A | 59 | −4.736 | 37.261 | 55.113 | 1.00 | 27.05 | A |
| ATOM | 391 | C | ASN | A | 59 | −8.351 | 39.631 | 52.714 | 1.00 | 28.83 | A |
| ATOM | 392 | O | ASN | A | 59 | −9.052 | 39.823 | 53.697 | 1.00 | 30.01 | A |
| ATOM | 393 | N | GLU | A | 60 | −8.507 | 40.326 | 51.597 | 1.00 | 30.40 | A |
| ATOM | 394 | CA | GLU | A | 60 | −9.534 | 41.348 | 51.507 | 1.00 | 31.99 | A |
| ATOM | 395 | CB | GLU | A | 60 | −8.966 | 42.637 | 50.945 | 1.00 | 30.74 | A |
| ATOM | 396 | CG | GLU | A | 60 | −7.958 | 43.296 | 51.844 | 1.00 | 33.45 | A |
| ATOM | 397 | CD | GLU | A | 60 | −7.447 | 44.568 | 51.220 | 1.00 | 34.00 | A |
| ATOM | 398 | OE1 | GLU | A | 60 | −8.260 | 45.494 | 51.027 | 1.00 | 37.35 | A |
| ATOM | 399 | OE2 | GLU | A | 60 | −6.248 | 44.643 | 50.915 | 1.00 | 32.39 | A |
| ATOM | 400 | C | GLU | A | 60 | −10.625 | 40.843 | 50.595 | 1.00 | 32.78 | A |
| ATOM | 401 | O | GLU | A | 60 | −10.363 | 40.126 | 49.620 | 1.00 | 33.16 | A |
| ATOM | 402 | N | LYS | A | 61 | −11.861 | 41.206 | 50.907 | 1.00 | 33.50 | A |
| ATOM | 403 | CA | LYS | A | 61 | −12.979 | 40.784 | 50.080 | 1.00 | 34.01 | A |
| ATOM | 404 | CB | LYS | A | 61 | −14.288 | 41.055 | 50.818 | 1.00 | 36.55 | A |
| ATOM | 405 | CG | LYS | A | 61 | −15.504 | 40.449 | 50.141 | 1.00 | 41.45 | A |
| ATOM | 406 | CD | LYS | A | 61 | −16.787 | 40.806 | 50.892 | 1.00 | 44.29 | A |
| ATOM | 407 | CE | LYS | A | 61 | −18.013 | 40.305 | 50.153 | 1.00 | 44.74 | A |
| ATOM | 408 | NZ | LYS | A | 61 | −19.239 | 40.572 | 50.965 | 1.00 | 47.69 | A |
| ATOM | 409 | C | LYS | A | 61 | −12.906 | 41.578 | 48.775 | 1.00 | 33.44 | A |
| ATOM | 410 | O | LYS | A | 61 | −12.568 | 42.769 | 48.784 | 1.00 | 33.86 | A |
| ATOM | 411 | N | LEU | A | 62 | −13.187 | 40.927 | 47.654 | 1.00 | 32.05 | A |
| ATOM | 412 | CA | LEU | A | 62 | −13.142 | 41.607 | 46.371 | 1.00 | 33.14 | A |
| ATOM | 413 | CB | LEU | A | 62 | −12.623 | 40.678 | 45.258 | 1.00 | 32.95 | A |
| ATOM | 414 | CG | LEU | A | 62 | −11.187 | 40.165 | 45.421 | 1.00 | 35.06 | A |
| ATOM | 415 | CD1 | LEU | A | 62 | −10.804 | 39.335 | 44.193 | 1.00 | 35.29 | A |
| ATOM | 416 | CD2 | LEU | A | 62 | −10.228 | 41.344 | 45.606 | 1.00 | 34.89 | A |
| ATOM | 417 | C | LEU | A | 62 | −14.526 | 42.093 | 45.977 | 1.00 | 33.44 | A |
| ATOM | 418 | O | LEU | A | 62 | −15.513 | 41.366 | 46.115 | 1.00 | 34.29 | A |
| ATOM | 419 | N | PRO | A | 63 | −14.626 | 43.342 | 45.506 | 1.00 | 33.07 | A |
| ATOM | 420 | CD | PRO | A | 63 | −13.582 | 44.368 | 45.347 | 1.00 | 32.27 | A |
| ATOM | 421 | CA | PRO | A | 63 | −15.944 | 43.839 | 45.103 | 1.00 | 32.15 | A |
| ATOM | 422 | CB | PRO | A | 63 | −15.681 | 45.309 | 44.761 | 1.00 | 32.76 | A |
| ATOM | 423 | CG | PRO | A | 63 | −14.223 | 45.326 | 44.357 | 1.00 | 32.85 | A |
| ATOM | 424 | C | PRO | A | 63 | −16.410 | 43.017 | 43.902 | 1.00 | 32.47 | A |
| ATOM | 425 | O | PRO | A | 63 | −15.588 | 42.416 | 43.177 | 1.00 | 31.14 | A |
| ATOM | 426 | N | GLU | A | 64 | −17.721 | 42.968 | 43.685 | 1.00 | 31.52 | A |
| ATOM | 427 | CA | GLU | A | 64 | −18.254 | 42.189 | 42.569 | 1.00 | 32.30 | A |
| ATOM | 428 | CB | GLU | A | 64 | −19.790 | 42.237 | 42.536 | 1.00 | 36.47 | A |
| ATOM | 429 | CG | GLU | A | 64 | −20.457 | 41.249 | 43.475 | 1.00 | 41.60 | A |
| ATOM | 430 | CD | GLU | A | 64 | −19.936 | 39.825 | 43.289 | 1.00 | 44.51 | A |
| ATOM | 431 | OE1 | GLU | A | 64 | −20.046 | 39.283 | 42.162 | 1.00 | 46.75 | A |
| ATOM | 432 | OE2 | GLU | A | 64 | −19.417 | 39.258 | 44.279 | 1.00 | 45.99 | A |
| ATOM | 433 | C | GLU | A | 64 | −17.752 | 42.548 | 41.185 | 1.00 | 29.49 | A |
| ATOM | 434 | O | GLU | A | 64 | −17.537 | 41.660 | 40.359 | 1.00 | 26.90 | A |
| ATOM | 435 | N | TYR | A | 65 | −17.577 | 43.836 | 40.905 | 1.00 | 27.49 | A |
| ATOM | 436 | CA | TYR | A | 65 | −17.138 | 44.200 | 39.565 | 1.00 | 27.12 | A |
| ATOM | 437 | CB | TYR | A | 65 | −17.216 | 45.715 | 39.368 | 1.00 | 27.10 | A |
| ATOM | 438 | CG | TYR | A | 65 | −16.285 | 46.550 | 40.214 | 1.00 | 27.17 | A |
| ATOM | 439 | CD1 | TYR | A | 65 | −14.975 | 46.816 | 39.803 | 1.00 | 25.51 | A |
| ATOM | 440 | CE1 | TYR | A | 65 | −14.143 | 47.621 | 40.558 | 1.00 | 27.31 | A |
| ATOM | 441 | CD2 | TYR | A | 65 | −16.731 | 47.109 | 41.411 | 1.00 | 26.81 | A |
| ATOM | 442 | CE2 | TYR | A | 65 | −15.911 | 47.904 | 42.174 | 1.00 | 28.77 | A |
| ATOM | 443 | CZ | TYR | A | 65 | −14.627 | 48.160 | 41.745 | 1.00 | 29.82 | A |
| ATOM | 444 | OH | TYR | A | 65 | −13.844 | 48.979 | 42.508 | 1.00 | 33.48 | A |
| ATOM | 445 | C | TYR | A | 65 | −15.736 | 43.661 | 39.220 | 1.00 | 24.82 | A |
| ATOM | 446 | O | TYR | A | 65 | −15.431 | 43.405 | 38.044 | 1.00 | 23.19 | A |
| ATOM | 447 | N | ILE | A | 66 | −14.892 | 43.481 | 40.235 | 1.00 | 25.20 | A |
| ATOM | 448 | CA | ILE | A | 66 | −13.556 | 42.922 | 40.007 | 1.00 | 26.39 | A |
| ATOM | 449 | CB | ILE | A | 66 | −12.598 | 43.255 | 41.181 | 1.00 | 26.58 | A |
| ATOM | 450 | CG2 | ILE | A | 66 | −11.315 | 42.405 | 41.087 | 1.00 | 27.43 | A |
| ATOM | 451 | CG1 | ILE | A | 66 | −12.227 | 44.749 | 41.114 | 1.00 | 29.08 | A |
| ATOM | 452 | CD1 | ILE | A | 66 | −11.185 | 45.175 | 42.112 | 1.00 | 30.16 | A |
| ATOM | 453 | C | ILE | A | 66 | −13.678 | 41.397 | 39.810 | 1.00 | 26.68 | A |
| ATOM | 454 | O | ILE | A | 66 | −13.010 | 40.823 | 38.956 | 1.00 | 27.11 | A |
| ATOM | 455 | N | LYS | A | 67 | −14.533 | 40.745 | 40.591 | 1.00 | 27.91 | A |
| ATOM | 456 | CA | LYS | A | 67 | −14.740 | 39.306 | 40.437 | 1.00 | 29.38 | A |
| ATOM | 457 | CB | LYS | A | 67 | −15.749 | 38.786 | 41.465 | 1.00 | 30.38 | A |
| ATOM | 458 | CG | LYS | A | 67 | −15.295 | 38.884 | 42.902 | 1.00 | 32.38 | A |
| ATOM | 459 | CD | LYS | A | 67 | −16.330 | 38.263 | 43.845 | 1.00 | 36.43 | A |
| ATOM | 460 | CE | LYS | A | 67 | −15.836 | 38.315 | 45.289 | 1.00 | 40.14 | A |
| ATOM | 461 | NZ | LYS | A | 67 | −16.840 | 37.774 | 46.267 | 1.00 | 44.58 | A |
| ATOM | 462 | C | LYS | A | 67 | −15.252 | 38.987 | 39.019 | 1.00 | 29.85 | A |
| ATOM | 463 | O | LYS | A | 67 | −14.776 | 38.052 | 38.383 | 1.00 | 28.01 | A |
| ATOM | 464 | N | GLN | A | 68 | −16.207 | 39.775 | 38.515 | 1.00 | 30.12 | A |
| ATOM | 465 | CA | GLN | A | 68 | −16.756 | 39.546 | 37.180 | 1.00 | 29.47 | A |
| ATOM | 466 | CB | GLN | A | 68 | −17.900 | 40.526 | 36.883 | 1.00 | 33.01 | A |
| ATOM | 467 | CG | GLN | A | 68 | −18.977 | 40.570 | 37.944 | 1.00 | 35.71 | A |
| ATOM | 468 | CD | GLN | A | 68 | −20.199 | 41.372 | 37.498 | 1.00 | 39.88 | A |
| ATOM | 469 | OE1 | GLN | A | 68 | −20.089 | 42.332 | 36.717 | 1.00 | 42.71 | A |
| ATOM | 470 | NE2 | GLN | A | 68 | −21.368 | 40.990 | 38.002 | 1.00 | 40.73 | A |
| ATOM | 471 | C | GLN | A | 68 | −15.686 | 39.691 | 36.103 | 1.00 | 29.74 | A |
| ATOM | 472 | O | GLN | A | 68 | −15.732 | 38.991 | 35.081 | 1.00 | 30.02 | A |
| ATOM | 473 | N | LYS | A | 69 | −14.734 | 40.611 | 36.297 | 1.00 | 26.78 | A |
| ATOM | 474 | CA | LYS | A | 69 | −13.669 | 40.769 | 35.311 | 1.00 | 26.10 | A |
| ATOM | 475 | CB | LYS | A | 69 | −12.981 | 42.137 | 35.439 | 1.00 | 23.05 | A |
| ATOM | 476 | CG | LYS | A | 69 | −13.695 | 43.222 | 34.592 | 1.00 | 21.87 | A |
| ATOM | 477 | CD | LYS | A | 69 | −13.365 | 44.666 | 35.037 | 1.00 | 19.54 | A |
| ATOM | 478 | CE | LYS | A | 69 | −14.012 | 45.686 | 34.081 | 1.00 | 20.65 | A |
| ATOM | 479 | NZ | LYS | A | 69 | −13.682 | 47.106 | 34.433 | 1.00 | 20.86 | A |
| ATOM | 480 | C | LYS | A | 69 | −12.658 | 39.619 | 35.450 | 1.00 | 24.70 | A |
| ATOM | 481 | O | LYS | A | 69 | −12.119 | 39.146 | 34.447 | 1.00 | 24.05 | A |
| ATOM | 482 | N | LEU | A | 70 | −12.420 | 39.162 | 36.680 | 1.00 | 26.08 | A |
| ATOM | 483 | CA | LEU | A | 70 | −11.507 | 38.025 | 36.914 | 1.00 | 27.98 | A |
| ATOM | 484 | CB | LEU | A | 70 | −11.383 | 37.719 | 38.411 | 1.00 | 27.23 | A |
| ATOM | 485 | CG | LEU | A | 70 | −10.489 | 38.642 | 39.228 | 1.00 | 28.37 | A |
| ATOM | 486 | CD1 | LEU | A | 70 | −10.655 | 38.372 | 40.720 | 1.00 | 28.59 | A |
| ATOM | 487 | CD2 | LEU | A | 70 | −9.052 | 38.415 | 38.773 | 1.00 | 26.77 | A |
| ATOM | 488 | C | LEU | A | 70 | −12.060 | 36.778 | 36.230 | 1.00 | 29.99 | A |
| ATOM | 489 | O | LEU | A | 70 | −11.314 | 35.981 | 35.666 | 1.00 | 31.87 | A |
| ATOM | 490 | N | GLN | A | 71 | −13.374 | 36.602 | 36.296 | 1.00 | 31.27 | A |
| ATOM | 491 | CA | GLN | A | 71 | −13.995 | 35.440 | 35.684 | 1.00 | 33.89 | A |
| ATOM | 492 | CB | GLN | A | 71 | −15.491 | 35.414 | 35.985 | 1.00 | 36.26 | A |
| ATOM | 493 | CG | GLN | A | 71 | −16.246 | 34.336 | 35.212 | 1.00 | 42.00 | A |
| ATOM | 494 | CD | GLN | A | 71 | −15.826 | 32.922 | 35.600 | 1.00 | 45.50 | A |
| ATOM | 495 | OE1 | GLN | A | 71 | −15.847 | 32.567 | 36.786 | 1.00 | 47.23 | A |
| ATOM | 496 | NE2 | GLN | A | 71 | −15.447 | 32.104 | 34.603 | 1.00 | 45.34 | A |
| ATOM | 497 | C | GLN | A | 71 | −13.777 | 35.386 | 34.181 | 1.00 | 34.10 | A |
| ATOM | 498 | O | GLN | A | 71 | −13.872 | 34.319 | 33.581 | 1.00 | 33.80 | A |
| ATOM | 499 | N | LEU | A | 72 | −13.486 | 36.526 | 33.561 | 1.00 | 32.87 | A |
| ATOM | 500 | CA | LEU | A | 72 | −13.271 | 36.533 | 32.119 | 1.00 | 32.81 | A |
| ATOM | 501 | CB | LEU | A | 72 | −13.268 | 37.974 | 31.574 | 1.00 | 30.82 | A |
| ATOM | 502 | CG | LEU | A | 72 | −14.599 | 38.759 | 31.635 | 1.00 | 30.29 | A |
| ATOM | 503 | CD1 | LEU | A | 72 | −14.395 | 40.206 | 31.148 | 1.00 | 27.81 | A |
| ATOM | 504 | CD2 | LEU | A | 72 | −15.638 | 38.047 | 30.772 | 1.00 | 30.00 | A |
| ATOM | 505 | C | LEU | A | 72 | −11.933 | 35.858 | 31.824 | 1.00 | 33.11 | A |
| ATOM | 506 | O | LEU | A | 72 | −11.645 | 35.490 | 30.684 | 1.00 | 31.50 | A |
| ATOM | 507 | N | LEU | A | 73 | −11.124 | 35.687 | 32.866 | 1.00 | 32.72 | A |
| ATOM | 508 | CA | LEU | A | 73 | −9.806 | 35.065 | 32.714 | 1.00 | 33.81 | A |
| ATOM | 509 | CB | LEU | A | 73 | −8.796 | 35.754 | 33.644 | 1.00 | 35.28 | A |
| ATOM | 510 | CG | LEU | A | 73 | −8.559 | 37.218 | 33.265 | 1.00 | 37.42 | A |
| ATOM | 511 | CD1 | LEU | A | 73 | −7.923 | 37.975 | 34.426 | 1.00 | 37.87 | A |
| ATOM | 512 | CD2 | LEU | A | 73 | −7.678 | 37.251 | 32.017 | 1.00 | 37.72 | A |
| ATOM | 513 | C | LEU | A | 73 | −9.781 | 33.558 | 32.963 | 1.00 | 32.63 | A |
| ATOM | 514 | O | LEU | A | 73 | −8.872 | 32.859 | 32.496 | 1.00 | 33.32 | A |
| ATOM | 515 | N | SER | A | 74 | −10.774 | 33.047 | 33.676 | 1.00 | 30.99 | A |
| ATOM | 516 | CA | SER | A | 74 | −10.802 | 31.617 | 33.980 | 1.00 | 29.90 | A |
| ATOM | 517 | CB | SER | A | 74 | −12.037 | 31.286 | 34.803 | 1.00 | 30.28 | A |
| ATOM | 518 | OG | SER | A | 74 | −12.151 | 32.197 | 35.893 | 1.00 | 37.30 | A |
| ATOM | 519 | C | SER | A | 74 | −10.782 | 30.751 | 32.719 | 1.00 | 29.33 | A |
| ATOM | 520 | O | SER | A | 74 | −11.611 | 30.928 | 31.811 | 1.00 | 27.15 | A |
| ATOM | 521 | N | SER | A | 75 | −9.868 | 29.781 | 32.676 | 1.00 | 27.21 | A |
| ATOM | 522 | CA | SER | A | 75 | −9.778 | 28.920 | 31.506 | 1.00 | 25.21 | A |
| ATOM | 523 | CB | SER | A | 75 | −9.041 | 29.638 | 30.379 | 1.00 | 26.98 | A |
| ATOM | 524 | OG | SER | A | 75 | −7.649 | 29.736 | 30.653 | 1.00 | 27.61 | A |
| ATOM | 525 | C | SER | A | 75 | −9.072 | 27.604 | 31.773 | 1.00 | 25.16 | A |
| ATOM | 526 | O | SER | A | 75 | −8.431 | 27.434 | 32.812 | 1.00 | 21.93 | A |
| ATOM | 527 | N | ILE | A | 76 | −9.213 | 26.686 | 30.816 | 1.00 | 24.41 | A |
| ATOM | 528 | CA | ILE | A | 76 | −8.588 | 25.358 | 30.855 | 1.00 | 24.95 | A |
| ATOM | 529 | CB | ILE | A | 76 | −9.603 | 24.222 | 30.750 | 1.00 | 26.86 | A |
| ATOM | 530 | CG2 | ILE | A | 76 | −8.850 | 22.885 | 30.655 | 1.00 | 27.48 | A |
| ATOM | 531 | CG1 | ILE | A | 76 | −10.605 | 24.295 | 31.888 | 1.00 | 29.55 | A |
| ATOM | 532 | CD1 | ILE | A | 76 | −10.016 | 24.020 | 33.204 | 1.00 | 33.33 | A |
| ATOM | 533 | C | ILE | A | 76 | −7.789 | 25.285 | 29.562 | 1.00 | 25.08 | A |
| ATOM | 534 | O | ILE | A | 76 | −8.340 | 25.534 | 28.481 | 1.00 | 24.07 | A |
| ATOM | 535 | N | LEU | A | 77 | −6.511 | 24.939 | 29.654 | 1.00 | 22.73 | A |
| ATOM | 536 | CA | LEU | A | 77 | −5.707 | 24.837 | 28.464 | 1.00 | 23.84 | A |
| ATOM | 537 | CB | LEU | A | 77 | −4.752 | 26.025 | 28.367 | 1.00 | 26.02 | A |
| ATOM | 538 | CG | LEU | A | 77 | −3.734 | 25.932 | 27.204 | 1.00 | 29.74 | A |
| ATOM | 539 | CD1 | LEU | A | 77 | −3.488 | 27.325 | 26.608 | 1.00 | 29.57 | A |
| ATOM | 540 | CD2 | LEU | A | 77 | −2.447 | 25.319 | 27.699 | 1.00 | 31.16 | A |
| ATOM | 541 | C | LEU | A | 77 | −4.916 | 23.532 | 28.513 | 1.00 | 23.98 | A |
| ATOM | 542 | O | LEU | A | 77 | −4.493 | 23.092 | 29.581 | 1.00 | 23.14 | A |
| ATOM | 543 | N | LEU | A | 78 | −4.769 | 22.897 | 27.361 | 1.00 | 23.73 | A |
| ATOM | 544 | CA | LEU | A | 78 | −3.982 | 21.671 | 27.239 | 1.00 | 25.08 | A |
| ATOM | 545 | CB | LEU | A | 78 | −4.906 | 20.463 | 27.072 | 1.00 | 26.86 | A |
| ATOM | 546 | CG | LEU | A | 78 | −5.700 | 19.996 | 28.287 | 1.00 | 28.53 | A |
| ATOM | 547 | CD1 | LEU | A | 78 | −6.688 | 18.904 | 27.894 | 1.00 | 30.02 | A |
| ATOM | 548 | CD2 | LEU | A | 78 | −4.715 | 19.457 | 29.319 | 1.00 | 31.81 | A |
| ATOM | 549 | C | LEU | A | 78 | −3.156 | 21.860 | 25.973 | 1.00 | 25.44 | A |
| ATOM | 550 | O | LEU | A | 78 | −3.714 | 22.194 | 24.930 | 1.00 | 25.36 | A |
| ATOM | 551 | N | MET | A | 79 | −1.839 | 21.674 | 26.055 | 1.00 | 24.42 | A |
| ATOM | 552 | CA | MET | A | 79 | −0.973 | 21.807 | 24.887 | 1.00 | 25.49 | A |
| ATOM | 553 | CB | MET | A | 79 | −0.155 | 23.100 | 24.950 | 1.00 | 27.60 | A |
| ATOM | 554 | CG | MET | A | 79 | 0.708 | 23.353 | 23.706 | 1.00 | 33.94 | A |
| ATOM | 555 | SD | MET | A | 79 | 1.544 | 24.995 | 23.654 | 1.00 | 38.48 | A |
| ATOM | 556 | CE | MET | A | 79 | 0.465 | 25.937 | 24.741 | 1.00 | 36.27 | A |
| ATOM | 557 | C | MET | A | 79 | −0.027 | 20.605 | 24.841 | 1.00 | 26.16 | A |
| ATOM | 558 | O | MET | A | 79 | 0.612 | 20.285 | 25.853 | 1.00 | 25.19 | A |
| ATOM | 559 | N | PHE | A | 80 | 0.061 | 19.949 | 23.680 | 1.00 | 26.66 | A |
| ATOM | 560 | CA | PHE | A | 80 | 0.919 | 18.767 | 23.497 | 1.00 | 28.28 | A |
| ATOM | 561 | CB | PHE | A | 80 | 0.076 | 17.509 | 23.219 | 1.00 | 29.89 | A |
| ATOM | 562 | CG | PHE | A | 80 | −1.082 | 17.326 | 24.154 | 1.00 | 31.47 | A |
| ATOM | 563 | CD1 | PHE | A | 80 | −2.205 | 18.152 | 24.066 | 1.00 | 34.23 | A |
| ATOM | 564 | CD2 | PHE | A | 80 | −1.036 | 16.358 | 25.152 | 1.00 | 33.79 | A |
| ATOM | 565 | CE1 | PHE | A | 80 | −3.269 | 18.019 | 24.968 | 1.00 | 34.34 | A |
| ATOM | 566 | CE2 | PHE | A | 80 | −2.098 | 16.216 | 26.064 | 1.00 | 34.66 | A |
| ATOM | 567 | CZ | PHE | A | 80 | −3.207 | 17.050 | 25.967 | 1.00 | 34.41 | A |
| ATOM | 568 | C | PHE | A | 80 | 1.862 | 18.921 | 22.309 | 1.00 | 29.99 | A |
| ATOM | 569 | O | PHE | A | 80 | 1.463 | 19.446 | 21.271 | 1.00 | 26.03 | A |
| ATOM | 570 | N | SER | A | 81 | 3.099 | 18.444 | 22.447 | 1.00 | 31.47 | A |
| ATOM | 571 | CA | SER | A | 81 | 4.034 | 18.465 | 21.325 | 1.00 | 35.92 | A |
| ATOM | 572 | CB | SER | A | 81 | 5.431 | 18.022 | 21.764 | 1.00 | 36.10 | A |
| ATOM | 573 | OG | SER | A | 81 | 5.974 | 18.945 | 22.705 | 1.00 | 40.41 | A |
| ATOM | 574 | C | SER | A | 81 | 3.453 | 17.425 | 20.354 | 1.00 | 37.84 | A |
| ATOM | 575 | O | SER | A | 81 | 3.029 | 16.350 | 20.778 | 1.00 | 38.67 | A |
| ATOM | 576 | N | ASN | A | 82 | 3.421 | 17.743 | 19.063 | 1.00 | 39.87 | A |
| ATOM | 577 | CA | ASN | A | 82 | 2.859 | 16.837 | 18.059 | 1.00 | 42.79 | A |
| ATOM | 578 | CB | ASN | A | 82 | 1.668 | 17.530 | 17.374 | 1.00 | 44.12 | A |
| ATOM | 579 | CG | ASN | A | 82 | 0.881 | 16.602 | 16.452 | 1.00 | 44.62 | A |
| ATOM | 580 | OD1 | ASN | A | 82 | 0.133 | 17.065 | 15.584 | 1.00 | 46.36 | A |
| ATOM | 581 | ND2 | ASN | A | 82 | 1.032 | 15.297 | 16.643 | 1.00 | 44.93 | A |
| ATOM | 582 | C | ASN | A | 82 | 3.930 | 16.466 | 17.019 | 1.00 | 45.11 | A |
| ATOM | 583 | O | ASN | A | 82 | 3.742 | 16.760 | 15.809 | 1.00 | 45.06 | A |
| ATOM | 584 | OXT | ASN | A | 82 | 4.964 | 15.892 | 17.439 | 1.00 | 48.72 | A |
| ATOM | 585 | CB | SER | B | 8 | −20.703 | 44.768 | 26.853 | 1.00 | 46.84 | B |
| ATOM | 586 | OG | SER | B | 8 | −20.236 | 45.831 | 26.037 | 1.00 | 49.95 | B |
| ATOM | 587 | C | SER | B | 8 | −18.436 | 43.671 | 26.952 | 1.00 | 44.17 | B |
| ATOM | 588 | O | SER | B | 8 | −17.598 | 43.937 | 26.079 | 1.00 | 45.41 | B |
| ATOM | 589 | N | SER | B | 8 | −20.548 | 42.345 | 27.331 | 1.00 | 46.65 | B |
| ATOM | 590 | CA | SER | B | 8 | −19.923 | 43.475 | 26.579 | 1.00 | 45.64 | B |
| ATOM | 591 | N | VAL | B | 9 | −18.112 | 43.548 | 28.239 | 1.00 | 40.04 | B |
| ATOM | 592 | CA | VAL | B | 9 | −16.731 | 43.710 | 28.703 | 1.00 | 35.61 | B |
| ATOM | 593 | CB | VAL | B | 9 | −16.655 | 43.761 | 30.262 | 1.00 | 35.13 | B |
| ATOM | 594 | CG1 | VAL | B | 9 | −15.189 | 43.763 | 30.721 | 1.00 | 32.66 | B |
| ATOM | 595 | CG2 | VAL | B | 9 | −17.358 | 45.018 | 30.785 | 1.00 | 33.73 | B |
| ATOM | 596 | C | VAL | B | 9 | −15.886 | 42.534 | 28.230 | 1.00 | 33.45 | B |
| ATOM | 597 | O | VAL | B | 9 | −16.322 | 41.401 | 28.329 | 1.00 | 31.66 | B |
| ATOM | 598 | N | PHE | B | 10 | −14.693 | 42.787 | 27.695 | 1.00 | 31.66 | B |
| ATOM | 599 | CA | PHE | B | 10 | −13.846 | 41.674 | 27.283 | 1.00 | 31.75 | B |
| ATOM | 600 | CB | PHE | B | 10 | −14.188 | 41.199 | 25.846 | 1.00 | 33.57 | B |
| ATOM | 601 | CG | PHE | B | 10 | −13.981 | 42.242 | 24.765 | 1.00 | 38.37 | B |
| ATOM | 602 | CD1 | PHE | B | 10 | −12.728 | 42.423 | 24.180 | 1.00 | 39.12 | B |
| ATOM | 603 | CD2 | PHE | B | 10 | −15.038 | 43.053 | 24.342 | 1.00 | 39.71 | B |
| ATOM | 604 | CE1 | PHE | B | 10 | −12.519 | 43.397 | 23.192 | 1.00 | 39.97 | B |
| ATOM | 605 | CE2 | PHE | B | 10 | −14.840 | 44.036 | 23.352 | 1.00 | 40.15 | B |
| ATOM | 606 | CZ | PHE | B | 10 | −13.578 | 44.207 | 22.779 | 1.00 | 40.06 | B |
| ATOM | 607 | C | PHE | B | 10 | −12.354 | 41.958 | 27.429 | 1.00 | 29.89 | B |
| ATOM | 608 | O | PHE | B | 10 | −11.918 | 43.123 | 27.519 | 1.00 | 26.55 | B |
| ATOM | 609 | N | VAL | B | 11 | −11.576 | 40.876 | 27.496 | 1.00 | 28.69 | B |
| ATOM | 610 | CA | VAL | B | 11 | −10.127 | 40.985 | 27.617 | 1.00 | 27.06 | B |
| ATOM | 611 | CB | VAL | B | 11 | −9.458 | 39.616 | 27.905 | 1.00 | 26.64 | B |
| ATOM | 612 | CG1 | VAL | B | 11 | −7.917 | 39.793 | 27.932 | 1.00 | 25.73 | B |
| ATOM | 613 | CG2 | VAL | B | 11 | −9.940 | 39.068 | 29.255 | 1.00 | 27.02 | B |
| ATOM | 614 | C | VAL | B | 11 | −9.587 | 41.497 | 26.307 | 1.00 | 26.17 | B |
| ATOM | 615 | O | VAL | B | 11 | −9.975 | 41.006 | 25.249 | 1.00 | 27.06 | B |
| ATOM | 616 | N | LYS | B | 12 | −8.692 | 42.479 | 26.358 | 1.00 | 25.60 | B |
| ATOM | 617 | CA | LYS | B | 12 | −8.135 | 43.006 | 25.129 | 1.00 | 27.71 | B |
| ATOM | 618 | CB | LYS | B | 12 | −8.219 | 44.525 | 25.107 | 1.00 | 30.24 | B |
| ATOM | 619 | CG | LYS | B | 12 | −8.134 | 45.080 | 23.703 | 1.00 | 35.27 | B |
| ATOM | 620 | CD | LYS | B | 12 | −7.894 | 46.579 | 23.697 | 1.00 | 38.59 | B |
| ATOM | 621 | CE | LYS | B | 12 | −7.611 | 47.051 | 22.274 | 1.00 | 38.61 | B |
| ATOM | 622 | NZ | LYS | B | 12 | −7.069 | 48.441 | 22.283 | 1.00 | 43.07 | B |
| ATOM | 623 | C | LYS | B | 12 | −6.682 | 42.586 | 24.985 | 1.00 | 27.43 | B |
| ATOM | 624 | O | LYS | B | 12 | −6.268 | 42.098 | 23.931 | 1.00 | 25.99 | B |
| ATOM | 625 | N | ASN | B | 13 | −5.913 | 42.801 | 26.048 | 1.00 | 25.85 | B |
| ATOM | 626 | CA | ASN | B | 13 | −4.502 | 42.446 | 26.073 | 1.00 | 25.52 | B |
| ATOM | 627 | CB | ASN | B | 13 | −3.628 | 43.698 | 26.042 | 1.00 | 26.55 | B |
| ATOM | 628 | CG | ASN | B | 13 | −3.987 | 44.627 | 24.882 | 1.00 | 29.83 | B |
| ATOM | 629 | OD1 | ASN | B | 13 | −4.218 | 44.170 | 23.762 | 1.00 | 28.51 | B |
| ATOM | 630 | ND2 | ASN | B | 13 | −4.019 | 45.929 | 25.142 | 1.00 | 28.66 | B |
| ATOM | 631 | C | ASN | B | 13 | −4.256 | 41.686 | 27.369 | 1.00 | 25.56 | B |
| ATOM | 632 | O | ASN | B | 13 | −4.968 | 41.885 | 28.377 | 1.00 | 22.74 | B |
| ATOM | 633 | N | VAL | B | 14 | −3.272 | 40.794 | 27.337 | 1.00 | 23.42 | B |
| ATOM | 634 | CA | VAL | B | 14 | −2.913 | 40.009 | 28.515 | 1.00 | 24.09 | B |
| ATOM | 635 | CB | VAL | B | 14 | −3.700 | 38.712 | 28.574 | 1.00 | 25.31 | B |
| ATOM | 636 | CG1 | VAL | B | 14 | −3.464 | 37.902 | 27.320 | 1.00 | 28.72 | B |
| ATOM | 637 | CG2 | VAL | B | 14 | −3.270 | 37.907 | 29.789 | 1.00 | 28.09 | B |
| ATOM | 638 | C | VAL | B | 14 | −1.426 | 39.700 | 28.432 | 1.00 | 25.36 | B |
| ATOM | 639 | O | VAL | B | 14 | −0.856 | 39.590 | 27.329 | 1.00 | 23.77 | B |
| ATOM | 640 | N | GLY | B | 15 | −0.781 | 39.605 | 29.583 | 1.00 | 24.37 | B |
| ATOM | 641 | CA | GLY | B | 15 | 0.633 | 39.296 | 29.579 | 1.00 | 26.32 | B |
| ATOM | 642 | C | GLY | B | 15 | 1.067 | 38.766 | 30.932 | 1.00 | 26.65 | B |
| ATOM | 643 | O | GLY | B | 15 | 0.325 | 38.856 | 31.921 | 1.00 | 23.77 | B |
| ATOM | 644 | N | TRP | B | 16 | 2.251 | 38.169 | 30.975 | 1.00 | 24.82 | B |
| ATOM | 645 | CA | TRP | B | 16 | 2.774 | 37.685 | 32.243 | 1.00 | 24.33 | B |
| ATOM | 646 | CB | TRP | B | 16 | 2.146 | 36.340 | 32.618 | 1.00 | 24.57 | B |
| ATOM | 647 | CG | TRP | B | 16 | 2.571 | 35.187 | 31.758 | 1.00 | 28.68 | B |
| ATOM | 648 | CD2 | TRP | B | 16 | 1.707 | 34.286 | 31.068 | 1.00 | 29.83 | B |
| ATOM | 649 | CE2 | TRP | B | 16 | 2.523 | 33.288 | 30.474 | 1.00 | 32.16 | B |
| ATOM | 650 | CE3 | TRP | B | 16 | 0.319 | 34.223 | 30.885 | 1.00 | 31.66 | B |
| ATOM | 651 | CD1 | TRP | B | 16 | 3.849 | 34.720 | 31.563 | 1.00 | 28.36 | B |
| ATOM | 652 | NE1 | TRP | B | 16 | 3.826 | 33.572 | 30.797 | 1.00 | 30.79 | B |
| ATOM | 653 | CZ2 | TRP | B | 16 | 1.989 | 32.234 | 29.719 | 1.00 | 32.79 | B |
| ATOM | 654 | CZ3 | TRP | B | 16 | −0.209 | 33.176 | 30.136 | 1.00 | 32.52 | B |
| ATOM | 655 | CH2 | TRP | B | 16 | 0.624 | 32.200 | 29.561 | 1.00 | 33.65 | B |
| ATOM | 656 | C | TRP | B | 16 | 4.286 | 37.553 | 32.173 | 1.00 | 22.17 | B |
| ATOM | 657 | O | TRP | B | 16 | 4.885 | 37.616 | 31.103 | 1.00 | 19.67 | B |
| ATOM | 658 | N | ALA | B | 17 | 4.896 | 37.406 | 33.335 | 1.00 | 20.00 | B |
| ATOM | 659 | CA | ALA | B | 17 | 6.330 | 37.226 | 33.431 | 1.00 | 20.97 | B |
| ATOM | 660 | CB | ALA | B | 17 | 7.008 | 38.549 | 33.720 | 1.00 | 19.52 | B |
| ATOM | 661 | C | ALA | B | 17 | 6.429 | 36.296 | 34.629 | 1.00 | 22.18 | B |
| ATOM | 662 | O | ALA | B | 17 | 5.936 | 36.629 | 35.707 | 1.00 | 20.03 | B |
| ATOM | 663 | N | THR | B | 18 | 6.994 | 35.106 | 34.442 | 1.00 | 21.90 | B |
| ATOM | 664 | CA | THR | B | 18 | 7.117 | 34.183 | 35.556 | 1.00 | 23.51 | B |
| ATOM | 665 | CB | THR | B | 18 | 6.289 | 32.899 | 35.317 | 1.00 | 24.62 | B |
| ATOM | 666 | OG1 | THR | B | 18 | 6.757 | 32.243 | 34.135 | 1.00 | 24.95 | B |
| ATOM | 667 | CG2 | THR | B | 18 | 4.788 | 33.231 | 35.129 | 1.00 | 24.61 | B |
| ATOM | 668 | C | THR | B | 18 | 8.600 | 33.817 | 35.753 | 1.00 | 25.91 | B |
| ATOM | 669 | O | THR | B | 18 | 9.398 | 33.852 | 34.797 | 1.00 | 22.59 | B |
| ATOM | 670 | N | GLN | B | 19 | 8.973 | 33.520 | 36.992 | 1.00 | 26.80 | B |
| ATOM | 671 | CA | GLN | B | 19 | 10.346 | 33.130 | 37.281 | 1.00 | 33.83 | B |
| ATOM | 672 | CB | GLN | B | 19 | 10.931 | 33.946 | 38.435 | 1.00 | 36.03 | B |
| ATOM | 673 | CG | GLN | B | 19 | 10.967 | 35.451 | 38.198 | 1.00 | 40.64 | B |
| ATOM | 674 | CD | GLN | B | 19 | 9.580 | 36.084 | 38.255 | 1.00 | 44.69 | B |
| ATOM | 675 | OE1 | GLN | B | 19 | 8.731 | 35.668 | 39.061 | 1.00 | 47.37 | B |
| ATOM | 676 | NE2 | GLN | B | 19 | 9.342 | 37.100 | 37.412 | 1.00 | 45.52 | B |
| ATOM | 677 | C | GLN | B | 19 | 10.348 | 31.654 | 37.648 | 1.00 | 36.07 | B |
| ATOM | 678 | O | GLN | B | 19 | 9.979 | 30.813 | 36.824 | 1.00 | 40.72 | B |
| ATOM | 679 | N | LEU | B | 20 | 10.741 | 31.311 | 38.867 | 1.00 | 35.52 | B |
| ATOM | 680 | CA | LEU | B | 20 | 10.780 | 29.900 | 39.225 | 1.00 | 33.73 | B |
| ATOM | 681 | CB | LEU | B | 20 | 11.898 | 29.628 | 40.241 | 1.00 | 36.64 | B |
| ATOM | 682 | CG | LEU | B | 20 | 12.300 | 28.147 | 40.301 | 1.00 | 38.88 | B |
| ATOM | 683 | CD1 | LEU | B | 20 | 13.121 | 27.835 | 39.050 | 1.00 | 40.32 | B |
| ATOM | 684 | CD2 | LEU | B | 20 | 13.121 | 27.843 | 41.542 | 1.00 | 41.16 | B |
| ATOM | 685 | C | LEU | B | 20 | 9.438 | 29.446 | 39.793 | 1.00 | 32.93 | B |
| ATOM | 686 | O | LEU | B | 20 | 8.748 | 28.624 | 39.192 | 1.00 | 34.16 | B |
| ATOM | 687 | N | THR | B | 21 | 9.058 | 29.989 | 40.942 | 1.00 | 29.35 | B |
| ATOM | 688 | CA | THR | B | 21 | 7.788 | 29.614 | 41.572 | 1.00 | 30.06 | B |
| ATOM | 689 | CB | THR | B | 21 | 8.028 | 29.092 | 42.978 | 1.00 | 28.17 | B |
| ATOM | 690 | OG1 | THR | B | 21 | 8.805 | 30.063 | 43.689 | 1.00 | 30.80 | B |
| ATOM | 691 | CG2 | THR | B | 21 | 8.807 | 27.754 | 42.950 | 1.00 | 29.30 | B |
| ATOM | 692 | C | THR | B | 21 | 6.842 | 30.817 | 41.712 | 1.00 | 28.78 | B |
| ATOM | 693 | O | THR | B | 21 | 5.830 | 30.749 | 42.420 | 1.00 | 29.23 | B |
| ATOM | 694 | N | SER | B | 22 | 7.180 | 31.924 | 41.073 | 1.00 | 28.03 | B |
| ATOM | 695 | CA | SER | B | 22 | 6.337 | 33.098 | 41.213 | 1.00 | 27.21 | B |
| ATOM | 696 | CB | SER | B | 22 | 6.954 | 34.042 | 42.244 | 1.00 | 28.04 | B |
| ATOM | 697 | OG | SER | B | 22 | 8.181 | 34.542 | 41.757 | 1.00 | 32.79 | B |
| ATOM | 698 | C | SER | B | 22 | 6.150 | 33.798 | 39.904 | 1.00 | 25.42 | B |
| ATOM | 699 | O | SER | B | 22 | 6.892 | 33.552 | 38.952 | 1.00 | 23.76 | B |
| ATOM | 700 | N | GLY | B | 23 | 5.155 | 34.678 | 39.848 | 1.00 | 23.40 | B |
| ATOM | 701 | CA | GLY | B | 23 | 4.918 | 35.397 | 38.616 | 1.00 | 23.53 | B |
| ATOM | 702 | C | GLY | B | 23 | 3.945 | 36.540 | 38.790 | 1.00 | 22.85 | B |
| ATOM | 703 | O | GLY | B | 23 | 3.347 | 36.705 | 39.858 | 1.00 | 19.43 | B |
| ATOM | 704 | N | ALA | B | 24 | 3.803 | 37.336 | 37.734 | 1.00 | 22.99 | B |
| ATOM | 705 | CA | ALA | B | 24 | 2.872 | 38.442 | 37.729 | 1.00 | 24.00 | B |
| ATOM | 706 | CB | ALA | B | 24 | 3.630 | 39.768 | 37.827 | 1.00 | 23.97 | B |
| ATOM | 707 | C | ALA | B | 24 | 2.138 | 38.323 | 36.403 | 1.00 | 24.31 | B |
| ATOM | 708 | O | ALA | B | 24 | 2.732 | 37.942 | 35.377 | 1.00 | 23.13 | B |
| ATOM | 709 | N | VAL | B | 25 | 0.837 | 38.594 | 36.448 | 1.00 | 24.53 | B |
| ATOM | 710 | CA | VAL | B | 25 | −0.047 | 38.550 | 35.281 | 1.00 | 25.48 | B |
| ATOM | 711 | CB | VAL | B | 25 | −1.244 | 37.605 | 35.490 | 1.00 | 28.33 | B |
| ATOM | 712 | CG1 | VAL | B | 25 | −2.184 | 37.668 | 34.255 | 1.00 | 28.58 | B |
| ATOM | 713 | CG2 | VAL | B | 25 | −0.770 | 36.196 | 35.726 | 1.00 | 30.81 | B |
| ATOM | 714 | C | VAL | B | 25 | −0.650 | 39.936 | 35.120 | 1.00 | 26.05 | B |
| ATOM | 715 | O | VAL | B | 25 | −1.015 | 40.560 | 36.119 | 1.00 | 24.83 | B |
| ATOM | 716 | N | TRP | B | 26 | −0.737 | 40.416 | 33.884 | 1.00 | 24.31 | B |
| ATOM | 717 | CA | TRP | B | 26 | −1.322 | 41.719 | 33.591 | 1.00 | 26.24 | B |
| ATOM | 718 | CB | TRP | B | 26 | −0.268 | 42.640 | 32.978 | 1.00 | 30.96 | B |
| ATOM | 719 | CG | TRP | B | 26 | −0.844 | 43.770 | 32.197 | 1.00 | 37.86 | B |
| ATOM | 720 | CD2 | TRP | B | 26 | −0.868 | 43.901 | 30.761 | 1.00 | 40.27 | B |
| ATOM | 721 | CE2 | TRP | B | 26 | −1.479 | 45.149 | 30.460 | 1.00 | 41.09 | B |
| ATOM | 722 | CE3 | TRP | B | 26 | −0.429 | 43.085 | 29.702 | 1.00 | 41.59 | B |
| ATOM | 723 | CD1 | TRP | B | 26 | −1.435 | 44.909 | 32.695 | 1.00 | 39.70 | B |
| ATOM | 724 | NE1 | TRP | B | 26 | −1.817 | 45.744 | 31.650 | 1.00 | 41.13 | B |
| ATOM | 725 | CZ2 | TRP | B | 26 | −1.656 | 45.599 | 29.144 | 1.00 | 42.05 | B |
| ATOM | 726 | CZ3 | TRP | B | 26 | −0.606 | 43.533 | 28.393 | 1.00 | 42.03 | B |
| ATOM | 727 | CH2 | TRP | B | 26 | −1.215 | 44.784 | 28.128 | 1.00 | 42.46 | B |
| ATOM | 728 | C | TRP | B | 26 | −2.476 | 41.511 | 32.584 | 1.00 | 25.57 | B |
| ATOM | 729 | O | TRP | B | 26 | −2.385 | 40.669 | 31.664 | 1.00 | 21.95 | B |
| ATOM | 730 | N | VAL | B | 27 | −3.555 | 42.270 | 32.757 | 1.00 | 23.60 | B |
| ATOM | 731 | CA | VAL | B | 27 | −4.712 | 42.170 | 31.875 | 1.00 | 22.60 | B |
| ATOM | 732 | CB | VAL | B | 27 | −5.863 | 41.386 | 32.532 | 1.00 | 23.81 | B |
| ATOM | 733 | CG1 | VAL | B | 27 | −7.011 | 41.260 | 31.539 | 1.00 | 22.72 | B |
| ATOM | 734 | CG2 | VAL | B | 27 | −5.377 | 39.998 | 33.014 | 1.00 | 25.00 | B |
| ATOM | 735 | C | VAL | B | 27 | −5.278 | 43.558 | 31.583 | 1.00 | 24.68 | B |
| ATOM | 736 | O | VAL | B | 27 | −5.430 | 44.371 | 32.495 | 1.00 | 23.54 | B |
| ATOM | 737 | N | GLN | B | 28 | −5.590 | 43.829 | 30.322 | 1.00 | 24.45 | B |
| ATOM | 738 | CA | GLN | B | 28 | −6.203 | 45.100 | 29.949 | 1.00 | 26.46 | B |
| ATOM | 739 | CB | GLN | B | 28 | −5.369 | 45.830 | 28.913 | 1.00 | 30.46 | B |
| ATOM | 740 | CG | GLN | B | 28 | −4.608 | 46.992 | 29.461 | 1.00 | 38.64 | B |
| ATOM | 741 | CD | GLN | B | 28 | −3.935 | 47.795 | 28.361 | 1.00 | 43.10 | B |
| ATOM | 742 | OE1 | GLN | B | 28 | −4.569 | 48.156 | 27.365 | 1.00 | 46.00 | B |
| ATOM | 743 | NE2 | GLN | B | 28 | −2.649 | 48.088 | 28.540 | 1.00 | 44.70 | B |
| ATOM | 744 | C | GLN | B | 28 | −7.548 | 44.745 | 29.326 | 1.00 | 24.08 | B |
| ATOM | 745 | O | GLN | B | 28 | −7.620 | 43.900 | 28.430 | 1.00 | 24.15 | B |
| ATOM | 746 | N | PHE | B | 29 | −8.610 | 45.380 | 29.795 | 1.00 | 22.39 | B |
| ATOM | 747 | CA | PHE | B | 29 | −9.936 | 45.115 | 29.251 | 1.00 | 21.79 | B |
| ATOM | 748 | CB | PHE | B | 29 | −10.966 | 45.098 | 30.382 | 1.00 | 20.55 | B |
| ATOM | 749 | CG | PHE | B | 29 | −10.716 | 44.019 | 31.385 | 1.00 | 21.12 | B |
| ATOM | 750 | CD1 | PHE | B | 29 | −10.012 | 44.283 | 32.540 | 1.00 | 21.25 | B |
| ATOM | 751 | CD2 | PHE | B | 29 | −11.143 | 42.711 | 31.141 | 1.00 | 22.04 | B |
| ATOM | 752 | CE1 | PHE | B | 29 | −9.724 | 43.261 | 33.458 | 1.00 | 21.96 | B |
| ATOM | 753 | CE2 | PHE | B | 29 | −10.865 | 41.686 | 32.045 | 1.00 | 21.64 | B |
| ATOM | 754 | CZ | PHE | B | 29 | −10.155 | 41.959 | 33.208 | 1.00 | 22.73 | B |
| ATOM | 755 | C | PHE | B | 29 | −10.277 | 46.170 | 28.204 | 1.00 | 22.83 | B |
| ATOM | 756 | O | PHE | B | 29 | −9.622 | 47.216 | 28.129 | 1.00 | 23.60 | B |
| ATOM | 757 | N | ASN | B | 30 | −11.303 | 45.906 | 27.403 | 1.00 | 24.69 | B |
| ATOM | 758 | CA | ASN | B | 30 | −11.691 | 46.828 | 26.352 | 1.00 | 27.19 | B |
| ATOM | 759 | CB | ASN | B | 30 | −12.741 | 46.178 | 25.463 | 1.00 | 29.82 | B |
| ATOM | 760 | CG | ASN | B | 30 | −14.071 | 45.995 | 26.163 | 1.00 | 31.05 | B |
| ATOM | 761 | OD1 | ASN | B | 30 | −14.140 | 45.550 | 27.306 | 1.00 | 32.99 | B |
| ATOM | 762 | ND2 | ASN | B | 30 | −15.148 | 46.344 | 25.467 | 1.00 | 35.35 | B |
| ATOM | 763 | C | ASN | B | 30 | −12.205 | 48.162 | 26.892 | 1.00 | 27.09 | B |
| ATOM | 764 | O | ASN | B | 30 | −12.228 | 49.143 | 26.167 | 1.00 | 27.50 | B |
| ATOM | 765 | N | ASP | B | 31 | −12.593 | 48.212 | 28.164 | 1.00 | 27.50 | B |
| ATOM | 766 | CA | ASP | B | 31 | −13.098 | 49.471 | 28.728 | 1.00 | 28.13 | B |
| ATOM | 767 | CB | ASP | B | 31 | −14.125 | 49.216 | 29.852 | 1.00 | 24.72 | B |
| ATOM | 768 | CG | ASP | B | 31 | −13.528 | 48.516 | 31.050 | 1.00 | 24.30 | B |
| ATOM | 769 | OD1 | ASP | B | 31 | −12.329 | 48.150 | 31.021 | 1.00 | 22.63 | B |
| ATOM | 770 | OD2 | ASP | B | 31 | −14.263 | 48.331 | 32.037 | 1.00 | 24.53 | B |
| ATOM | 771 | C | ASP | B | 31 | −11.948 | 50.315 | 29.246 | 1.00 | 27.69 | B |
| ATOM | 772 | O | ASP | B | 31 | −12.161 | 51.369 | 29.837 | 1.00 | 28.05 | B |
| ATOM | 773 | N | GLY | B | 32 | −10.723 | 49.845 | 29.018 | 1.00 | 27.51 | B |
| ATOM | 774 | CA | GLY | B | 32 | −9.553 | 50.588 | 29.461 | 1.00 | 26.87 | B |
| ATOM | 775 | C | GLY | B | 32 | −9.062 | 50.281 | 30.875 | 1.00 | 25.54 | B |
| ATOM | 776 | O | GLY | B | 32 | −8.039 | 50.822 | 31.299 | 1.00 | 27.40 | B |
| ATOM | 111 | N | SER | B | 33 | −9.772 | 49.445 | 31.619 | 1.00 | 23.05 | B |
| ATOM | 778 | CA | SER | B | 33 | −9.317 | 49.130 | 32.972 | 1.00 | 22.45 | B |
| ATOM | 779 | CB | SER | B | 33 | −10.454 | 48.589 | 33.806 | 1.00 | 19.16 | B |
| ATOM | 780 | OG | SER | B | 33 | −10.999 | 47.395 | 33.238 | 1.00 | 20.75 | B |
| ATOM | 781 | C | SER | B | 33 | −8.187 | 48.104 | 32.884 | 1.00 | 21.94 | B |
| ATOM | 782 | O | SER | B | 33 | −8.013 | 47.454 | 31.828 | 1.00 | 20.92 | B |
| ATOM | 783 | N | GLN | B | 34 | −7.422 | 47.975 | 33.967 | 1.00 | 22.53 | B |
| ATOM | 784 | CA | GLN | B | 34 | −6.262 | 47.064 | 34.018 | 1.00 | 24.67 | B |
| ATOM | 785 | CB | GLN | B | 34 | −4.956 | 47.828 | 33.838 | 1.00 | 27.02 | B |
| ATOM | 786 | CG | GLN | B | 34 | −4.877 | 48.736 | 32.647 | 1.00 | 34.24 | B |
| ATOM | 787 | CD | GLN | B | 34 | −3.546 | 49.450 | 32.589 | 1.00 | 37.10 | B |
| ATOM | 788 | OE1 | GLN | B | 34 | −2.487 | 48.825 | 32.361 | 1.00 | 37.26 | B |
| ATOM | 789 | NE2 | GLN | B | 34 | −3.575 | 50.768 | 32.816 | 1.00 | 38.53 | B |
| ATOM | 790 | C | GLN | B | 34 | −6.127 | 46.350 | 35.358 | 1.00 | 25.48 | B |
| ATOM | 791 | O | GLN | B | 34 | −6.407 | 46.938 | 36.421 | 1.00 | 24.70 | B |
| ATOM | 792 | N | LEU | B | 35 | −5.685 | 45.095 | 35.304 | 1.00 | 22.93 | B |
| ATOM | 793 | CA | LEU | B | 35 | −5.435 | 44.284 | 36.505 | 1.00 | 23.57 | B |
| ATOM | 794 | CB | LEU | B | 35 | −6.301 | 43.016 | 36.507 | 1.00 | 24.87 | B |
| ATOM | 795 | CG | LEU | B | 35 | −7.740 | 43.074 | 36.983 | 1.00 | 26.23 | B |
| ATOM | 796 | CD1 | LEU | B | 35 | −8.511 | 41.841 | 36.512 | 1.00 | 26.00 | B |
| ATOM | 797 | CD2 | LEU | B | 35 | −7.750 | 43.187 | −38.513 | 1.00 | 23.79 | B |
| ATOM | 798 | C | LEU | B | 35 | −3.975 | 43.826 | 36.478 | 1.00 | 24.00 | B |
| ATOM | 799 | O | LEU | B | 35 | −3.497 | 43.342 | 35.445 | 1.00 | 22.60 | B |
| ATOM | 800 | N | VAL | B | 36 | −3.252 | 44.022 | 37.578 | 1.00 | 23.46 | B |
| ATOM | 801 | CA | VAL | B | 36 | −1.887 | 43.515 | 37.684 | 1.00 | 24.10 | B |
| ATOM | 802 | CB | VAL | B | 36 | −0.840 | 44.619 | 37.903 | 1.00 | 24.21 | B |
| ATOM | 803 | CG1 | VAL | B | 36 | 0.549 | 43.983 | 38.042 | 1.00 | 26.16 | B |
| ATOM | 804 | CG2 | VAL | B | 36 | −0.836 | 45.578 | 36.742 | 1.00 | 24.66 | B |
| ATOM | 805 | C | VAL | B | 36 | −1.969 | 42.614 | 38.923 | 1.00 | 25.01 | B |
| ATOM | 806 | O | VAL | B | 36 | −2.338 | 43.083 | 40.022 | 1.00 | 22.79 | B |
| ATOM | 807 | N | MET | B | 37 | −1.689 | 41.317 | 38.738 | 1.00 | 23.05 | B |
| ATOM | 808 | CA | MET | B | 37 | −1.783 | 40.340 | 39.829 | 1.00 | 22.68 | B |
| ATOM | 809 | CB | MET | B | 37 | −2.847 | 39.295 | 39.491 | 1.00 | 22.71 | B |
| ATOM | 810 | CG | MET | B | 37 | −3.996 | 39.933 | 38.756 | 1.00 | 26.04 | B |
| ATOM | 811 | SD | MET | B | 37 | −5.501 | 39.068 | 38.822 | 1.00 | 26.70 | B |
| ATOM | 812 | CE | MET | B | 37 | −5.327 | 37.927 | 37.565 | 1.00 | 26.34 | B |
| ATOM | 813 | C | MET | B | 37 | −0.478 | 39.629 | 40.099 | 1.00 | 22.50 | B |
| ATOM | 814 | O | MET | B | 37 | 0.183 | 39.160 | 39.164 | 1.00 | 23.17 | B |
| ATOM | 815 | N | GLN | B | 38 | −0.118 | 39.541 | 41.373 | 1.00 | 21.09 | B |
| ATOM | 816 | CA | GLN | B | 38 | 1.110 | 38.864 | 41.790 | 1.00 | 21.56 | B |
| ATOM | 817 | CB | GLN | B | 38 | 1.715 | 39.584 | 43.003 | 1.00 | 23.88 | B |
| ATOM | 818 | CG | GLN | B | 38 | 2.308 | 40.990 | 42.679 | 1.00 | 26.47 | B |
| ATOM | 819 | CD | GLN | B | 38 | 3.393 | 40.935 | 41.597 | 1.00 | 29.08 | B |
| ATOM | 820 | OE1 | GLN | B | 38 | 4.103 | 39.935 | 41.474 | 1.00 | 29.45 | B |
| ATOM | 821 | NE2 | GLN | B | 38 | 3.529 | 42.009 | 40.820 | 1.00 | 28.64 | B |
| ATOM | 822 | C | GLN | B | 38 | 0.629 | 37.462 | 42.177 | 1.00 | 21.49 | B |
| ATOM | 823 | O | GLN | B | 38 | −0.401 | 37.329 | 42.818 | 1.00 | 19.42 | B |
| ATOM | 824 | N | ALA | B | 39 | 1.371 | 36.426 | 41.805 | 1.00 | 19.11 | B |
| ATOM | 825 | CA | ALA | B | 39 | 0.946 | 35.067 | 42.089 | 1.00 | 20.63 | B |
| ATOM | 826 | CB | ALA | B | 39 | 0.194 | 34.483 | 40.874 | 1.00 | 20.95 | B |
| ATOM | 827 | C | ALA | B | 39 | 2.129 | 34.185 | 42.439 | 1.00 | 21.22 | B |
| ATOM | 828 | O | ALA | B | 39 | 3.292 | 34.548 | 42.211 | 1.00 | 20.65 | B |
| ATOM | 829 | N | GLY | B | 40 | 1.833 | 33.033 | 43.023 | 1.00 | 21.29 | B |
| ATOM | 830 | CA | GLY | B | 40 | 2.910 | 32.125 | 43.382 | 1.00 | 22.60 | B |
| ATOM | 831 | C | GLY | B | 40 | 2.422 | 30.706 | 43.549 | 1.00 | 22.77 | B |
| ATOM | 832 | O | GLY | B | 40 | 1.265 | 30.448 | 43.914 | 1.00 | 21.33 | B |
| ATOM | 833 | N | VAL | B | 41 | 3.322 | 29.772 | 43.270 | 1.00 | 23.45 | B |
| ATOM | 834 | CA | VAL | B | 41 | 3.037 | 28.359 | 43.424 | 1.00 | 22.65 | B |
| ATOM | 835 | CB | VAL | B | 41 | 4.088 | 27.540 | 42.681 | 1.00 | 23.98 | B |
| ATOM | 836 | CG1 | VAL | B | 41 | 3.925 | 26.056 | 42.983 | 1.00 | 23.94 | B |
| ATOM | 837 | CG2 | VAL | B | 41 | 3.935 | 27.803 | 41.183 | 1.00 | 25.98 | B |
| ATOM | 838 | C | VAL | B | 41 | 3.108 | 28.074 | 44.910 | 1.00 | 21.21 | B |
| ATOM | 839 | O | VAL | B | 41 | 4.054 | 28.466 | 45.557 | 1.00 | 20.44 | B |
| ATOM | 840 | N | SER | B | 42 | 2.111 | 27.384 | 45.453 | 1.00 | 22.01 | B |
| ATOM | 841 | CA | SER | B | 42 | 2.081 | 27.095 | 46.882 | 1.00 | 21.18 | B |
| ATOM | 842 | CB | SER | B | 42 | 0.741 | 27.559 | 47.448 | 1.00 | 19.17 | B |
| ATOM | 843 | OG | SER | B | 42 | −0.294 | 26.968 | 46.698 | 1.00 | 21.34 | B |
| ATOM | 844 | C | SER | B | 42 | 2.317 | 25.606 | 47.203 | 1.00 | 24.43 | B |
| ATOM | 845 | O | SER | B | 42 | 2.600 | 25.258 | 48.354 | 1.00 | 25.28 | B |
| ATOM | 846 | N | SER | B | 43 | 2.126 | 24.732 | 46.213 | 1.00 | 24.56 | B |
| ATOM | 847 | CA | SER | B | 43 | 2.411 | 23.296 | 46.374 | 1.00 | 24.94 | B |
| ATOM | 848 | CB | SER | B | 43 | 1.241 | 22.478 | 46.977 | 1.00 | 23.50 | B |
| ATOM | 849 | OG | SER | B | 43 | 0.016 | 22.631 | 46.302 | 1.00 | 28.57 | B |
| ATOM | 850 | C | SER | B | 43 | 2.849 | 22.699 | 45.043 | 1.00 | 26.29 | B |
| ATOM | 851 | O | SER | B | 43 | 2.355 | 23.069 | 43.969 | 1.00 | 24.41 | B |
| ATOM | 852 | N | ILE | B | 44 | 3.811 | 21.780 | 45.120 | 1.00 | 25.28 | B |
| ATOM | 853 | CA | ILE | B | 44 | 4.337 | 21.115 | 43.947 | 1.00 | 24.80 | B |
| ATOM | 854 | CB | ILE | B | 44 | 5.788 | 21.580 | 43.673 | 1.00 | 27.42 | B |
| ATOM | 855 | CG2 | ILE | B | 44 | 6.427 | 20.731 | 42.572 | 1.00 | 26.96 | B |
| ATOM | 856 | CG1 | ILE | B | 44 | 5.780 | 23.062 | 43.293 | 1.00 | 26.37 | B |
| ATOM | 857 | CD1 | ILE | B | 44 | 7.143 | 23.654 | 43.008 | 1.00 | 28.02 | B |
| ATOM | 858 | C | ILE | B | 44 | 4.285 | 19.617 | 44.201 | 1.00 | 26.01 | B |
| ATOM | 859 | O | ILE | B | 44 | 4.629 | 19.145 | 45.294 | 1.00 | 23.90 | B |
| ATOM | 860 | N | SER | B | 45 | 3.816 | 18.890 | 43.195 | 1.00 | 25.60 | B |
| ATOM | 861 | CA | SER | B | 45 | 3.685 | 17.448 | 43.257 | 1.00 | 25.92 | B |
| ATOM | 862 | CB | SER | B | 45 | 2.206 | 17.095 | 43.212 | 1.00 | 28.69 | B |
| ATOM | 863 | OG | SER | B | 45 | 1.981 | 15.697 | 43.280 | 1.00 | 33.00 | B |
| ATOM | 864 | C | SER | B | 45 | 4.440 | 16.891 | 42.044 | 1.00 | 26.07 | B |
| ATOM | 865 | O | SER | B | 45 | 3.989 | 17.043 | 40.895 | 1.00 | 26.72 | B |
| ATOM | 866 | N | TYR | B | 46 | 5.615 | 16.305 | 42.302 | 1.00 | 23.13 | B |
| ATOM | 867 | CA | TYR | B | 46 | 6.459 | 15.721 | 41.263 | 1.00 | 22.07 | B |
| ATOM | 868 | CB | TYR | B | 46 | 7.947 | 15.940 | 41.573 | 1.00 | 21.10 | B |
| ATOM | 869 | CG | TYR | B | 46 | 8.887 | 15.289 | 40.560 | 1.00 | 21.47 | B |
| ATOM | 870 | CD1 | TYR | B | 46 | 9.105 | 15.874 | 39.324 | 1.00 | 20.80 | B |
| ATOM | 871 | CE1 | TYR | B | 46 | 9.986 | 15.320 | 38.396 | 1.00 | 22.20 | B |
| ATOM | 872 | CD2 | TYR | B | 46 | 9.580 | 14.097 | 40.860 | 1.00 | 22.24 | B |
| ATOM | 873 | CE2 | TYR | B | 46 | 10.476 | 13.523 | 39.938 | 1.00 | 21.77 | B |
| ATOM | 874 | CZ | TYR | B | 46 | 10.668 | 14.147 | 38.704 | 1.00 | 22.48 | B |
| ATOM | 875 | OH | TYR | B | 46 | 11.518 | 13.618 | 37.763 | 1.00 | 22.53 | B |
| ATOM | 876 | C | TYR | B | 46 | 6.245 | 14.213 | 41.140 | 1.00 | 23.70 | B |
| ATOM | 877 | O | TYR | B | 46 | 6.366 | 13.468 | 42.127 | 1.00 | 24.10 | B |
| ATOM | 878 | N | THR | B | 47 | 5.949 | 13.751 | 39.935 | 1.00 | 22.63 | B |
| ATOM | 879 | CA | THR | B | 47 | 5.784 | 12.310 | 39.730 | 1.00 | 23.41 | B |
| ATOM | 880 | CB | THR | B | 47 | 4.451 | 11.990 | 39.079 | 1.00 | 23.58 | B |
| ATOM | 881 | OG1 | THR | B | 47 | 3.407 | 12.379 | 39.977 | 1.00 | 25.78 | B |
| ATOM | 882 | CG2 | THR | B | 47 | 4.332 | 10.478 | 38.800 | 1.00 | 24.55 | B |
| ATOM | 883 | C | THR | B | 47 | 6.913 | 11.866 | 38.821 | 1.00 | 21.48 | B |
| ATOM | 884 | O | THR | B | 47 | 7.002 | 12.317 | 37.679 | 1.00 | 21.40 | B |
| ATOM | 885 | N | SER | B | 48 | 7.786 | 11.005 | 39.340 | 1.00 | 20.62 | B |
| ATOM | 886 | CA | SER | B | 48 | 8.944 | 10.528 | 38.588 | 1.00 | 19.68 | B |
| ATOM | 887 | CB | SER | B | 48 | 9.837 | 9.668 | 39.480 | 1.00 | 19.79 | B |
| ATOM | 888 | OG | SER | B | 48 | 9.147 | 8.463 | 39.856 | 1.00 | 20.44 | B |
| ATOM | 889 | C | SER | B | 48 | 8.517 | 9.706 | 37.394 | 1.00 | 20.21 | B |
| ATOM | 890 | O | SER | B | 48 | 7.360 | 9.286 | 37.300 | 1.00 | 20.77 | B |
| ATOM | 891 | N | PRO | B | 49 | 9.453 | 9.429 | 36.475 | 1.00 | 20.80 | B |
| ATOM | 892 | CD | PRO | B | 49 | 10.839 | 9.921 | 36.382 | 1.00 | 19.25 | B |
| ATOM | 893 | CA | PRO | B | 49 | 9.108 | 8.635 | 35.293 | 1.00 | 21.42 | B |
| ATOM | 894 | CB | PRO | B | 49 | 10.434 | 8.535 | 34.547 | 1.00 | 19.67 | B |
| ATOM | 895 | CG | PRO | B | 49 | 11.072 | 9.870 | 34.879 | 1.00 | 19.88 | B |
| ATOM | 896 | C | PRO | B | 49 | 8.548 | 7.286 | 35.677 | 1.00 | 22.31 | B |
| ATOM | 897 | O | PRO | B | 49 | 7.734 | 6.724 | 34.938 | 1.00 | 22.96 | B |
| ATOM | 898 | N | ASP | B | 50 | 8.957 | 6.771 | 36.834 | 1.00 | 22.40 | B |
| ATOM | 899 | CA | ASP | B | 50 | 8.466 | 5.474 | 37.308 | 1.00 | 24.50 | B |
| ATOM | 900 | CB | ASP | B | 50 | 9.561 | 4.738 | 38.096 | 1.00 | 24.53 | B |
| ATOM | 901 | CG | ASP | B | 50 | 10.661 | 4.197 | 37.181 | 1.00 | 23.14 | B |
| ATOM | 902 | OD1 | ASP | B | 50 | 10.388 | 3.260 | 36.395 | 1.00 | 25.84 | B |
| ATOM | 903 | OD2 | ASP | B | 50 | 11.788 | 4.726 | 37.217 | 1.00 | 21.10 | B |
| ATOM | 904 | C | ASP | B | 50 | 7.167 | 5.537 | 38.134 | 1.00 | 26.54 | B |
| ATOM | 905 | O | ASP | B | 50 | 6.720 | 4.526 | 38.719 | 1.00 | 24.08 | B |
| ATOM | 906 | N | GLY | B | 51 | 6.558 | 6.722 | 38.180 | 1.00 | 25.82 | B |
| ATOM | 907 | CA | GLY | B | 51 | 5.291 | 6.856 | 38.873 | 1.00 | 26.67 | B |
| ATOM | 908 | C | GLY | B | 51 | 5.327 | 7.099 | 40.366 | 1.00 | 28.06 | B |
| ATOM | 909 | O | GLY | B | 51 | 4.319 | 6.892 | 41.031 | 1.00 | 30.08 | B |
| ATOM | 910 | N | GLN | B | 52 | 6.459 | 7.497 | 40.922 | 1.00 | 27.83 | B |
| ATOM | 911 | CA | GLN | B | 52 | 6.486 | 7.770 | 42.361 | 1.00 | 29.96 | B |
| ATOM | 912 | CB | GLN | B | 52 | 7.822 | 7.330 | 42.966 | 1.00 | 32.90 | B |
| ATOM | 913 | CG | GLN | B | 52 | 8.182 | 5.864 | 42.564 | 1.00 | 38.75 | B |
| ATOM | 914 | CD | GLN | B | 52 | 6.963 | 4.910 | 42.669 | 1.00 | 41.93 | B |
| ATOM | 915 | OE1 | GLN | B | 52 | 6.482 | 4.599 | 43.779 | 1.00 | 43.80 | B |
| ATOM | 916 | NE2 | GLN | B | 52 | 6.446 | 4.466 | 41.507 | 1.00 | 42.57 | B |
| ATOM | 917 | C | GLN | B | 52 | 6.262 | 9.279 | 42.581 | 1.00 | 28.82 | B |
| ATOM | 918 | O | GLN | B | 52 | 6.939 | 10.119 | 41.966 | 1.00 | 27.16 | B |
| ATOM | 919 | N | THR | B | 53 | 5.325 | 9.612 | 43.465 | 1.00 | 28.03 | B |
| ATOM | 920 | CA | THR | B | 53 | 4.991 | 11.021 | 43.733 | 1.00 | 27.41 | B |
| ATOM | 921 | CB | THR | B | 53 | 3.455 | 11.224 | 43.667 | 1.00 | 26.41 | B |
| ATOM | 922 | OG1 | THR | B | 53 | 3.014 | 10.878 | 42.347 | 1.00 | 24.55 | B |
| ATOM | 923 | CG2 | THR | B | 53 | 3.066 | 12.721 | 43.946 | 1.00 | 27.48 | B |
| ATOM | 924 | C | THR | B | 53 | 5.527 | 11.561 | 45.060 | 1.00 | 26.97 | B |
| ATOM | 925 | O | THR | B | 53 | 5.439 | 10.911 | 46.098 | 1.00 | 26.40 | B |
| ATOM | 926 | N | THR | B | 54 | 6.109 | 12.751 | 44.993 | 1.00 | 25.91 | B |
| ATOM | 927 | CA | THR | B | 54 | 6.654 | 13.435 | 46.148 | 1.00 | 25.43 | B |
| ATOM | 928 | CB | THR | B | 54 | 8.185 | 13.543 | 46.048 | 1.00 | 26.30 | B |
| ATOM | 929 | OG1 | THR | B | 54 | 8.731 | 12.217 | 46.017 | 1.00 | 27.51 | B |
| ATOM | 930 | CG2 | THR | B | 54 | 8.761 | 14.270 | 47.249 | 1.00 | 26.98 | B |
| ATOM | 931 | C | THR | B | 54 | 6.032 | 14.811 | 46.119 | 1.00 | 24.29 | B |
| ATOM | 932 | O | THR | B | 54 | 6.058 | 15.494 | 45.085 | 1.00 | 21.45 | B |
| ATOM | 933 | N | ARG | B | 55 | 5.450 | 15.202 | 47.244 | 1.00 | 24.59 | B |
| ATOM | 934 | CA | ARG | B | 55 | 4.791 | 16.498 | 47.343 | 1.00 | 28.29 | B |
| ATOM | 935 | CB | ARG | B | 55 | 3.382 | 16.307 | 47.906 | 1.00 | 30.45 | B |
| ATOM | 936 | CG | ARG | B | 55 | 2.547 | 15.395 | 47.020 | 1.00 | 36.62 | B |
| ATOM | 937 | CD | ARG | B | 55 | 1.134 | 15.165 | 47.550 | 1.00 | 40.16 | B |
| ATOM | 938 | NE | ARG | B | 55 | 0.425 | 14.191 | 46.728 | 1.00 | 42.96 | B |
| ATOM | 939 | CZ | ARG | B | 55 | 0.574 | 12.871 | 46.832 | 1.00 | 45.47 | B |
| ATOM | 940 | NH1 | ARG | B | 55 | 1.409 | 12.354 | 47.736 | 1.00 | 45.46 | B |
| ATOM | 941 | NH2 | ARG | B | 55 | −0.107 | 12.063 | 46.019 | 1.00 | 45.66 | B |
| ATOM | 942 | C | ARG | B | 55 | 5.565 | 17.488 | 48.195 | 1.00 | 27.52 | B |
| ATOM | 943 | O | ARG | B | 55 | 6.225 | 17.112 | 49.167 | 1.00 | 27.20 | B |
| ATOM | 944 | N | TYR | B | 56 | 5.513 | 18.754 | 47.803 | 1.00 | 27.30 | B |
| ATOM | 945 | CA | TYR | B | 56 | 6.194 | 19.794 | 48.548 | 1.00 | 28.29 | B |
| ATOM | 946 | CB | TYR | B | 56 | 7.414 | 20.312 | 47.804 | 1.00 | 29.22 | B |
| ATOM | 947 | CG | TYR | B | 56 | 8.406 | 19.234 | 47.446 | 1.00 | 33.48 | B |
| ATOM | 948 | CD1 | TYR | B | 56 | 8.220 | 18.443 | 46.307 | 1.00 | 32.53 | B |
| ATOM | 949 | CE1 | TYR | B | 56 | 9.167 | 17.490 | 45.932 | 1.00 | 35.51 | B |
| ATOM | 950 | CD2 | TYR | B | 56 | 9.560 | 19.035 | 48.216 | 1.00 | 33.59 | B |
| ATOM | 951 | CE2 | TYR | B | 56 | 10.514 | 18.073 | 47.853 | 1.00 | 37.05 | B |
| ATOM | 952 | CZ | TYR | B | 56 | 10.312 | 17.313 | 46.705 | 1.00 | 36.68 | B |
| ATOM | 953 | OH | TYR | B | 56 | 11.279 | 16.426 | 46.287 | 1.00 | 39.21 | B |
| ATOM | 954 | C | TYR | B | 56 | 5.242 | 20.943 | 48.771 | 1.00 | 28.88 | B |
| ATOM | 955 | O | TYR | B | 56 | 4.520 | 21.363 | 47.858 | 1.00 | 28.25 | B |
| ATOM | 956 | N | GLY | B | 57 | 5.228 | 21.408 | 50.014 | 1.00 | 28.70 | B |
| ATOM | 957 | CA | GLY | B | 57 | 4.407 | 22.524 | 50.412 | 1.00 | 26.92 | B |
| ATOM | 958 | C | GLY | B | 57 | 5.242 | 23.777 | 50.511 | 1.00 | 27.44 | B |
| ATOM | 959 | O | GLY | B | 57 | 6.460 | 23.759 | 50.327 | 1.00 | 25.48 | B |
| ATOM | 960 | N | GLU | B | 58 | 4.562 | 24.872 | 50.838 | 1.00 | 28.85 | B |
| ATOM | 961 | CA | GLU | B | 58 | 5.170 | 26.195 | 50.944 | 1.00 | 31.01 | B |
| ATOM | 962 | CB | GLU | B | 58 | 4.075 | 27.209 | 51.289 | 1.00 | 32.60 | B |
| ATOM | 963 | CG | GLU | B | 58 | 4.296 | 28.539 | 50.685 | 1.00 | 36.52 | B |
| ATOM | 964 | CD | GLU | B | 58 | 3.023 | 29.353 | 50.618 | 1.00 | 37.61 | B |
| ATOM | 965 | OE1 | GLU | B | 58 | 3.041 | 30.325 | 49.847 | 1.00 | 39.19 | B |
| ATOM | 966 | OE2 | GLU | B | 58 | 2.030 | 29.016 | 51.315 | 1.00 | 35.73 | B |
| ATOM | 967 | C | GLU | B | 58 | 6.289 | 26.297 | 51.961 | 1.00 | 29.67 | B |
| ATOM | 968 | O | GLU | B | 58 | 7.207 | 27.084 | 51.795 | 1.00 | 28.65 | B |
| ATOM | 969 | N | ASN | B | 59 | 6.207 | 25.500 | 53.017 | 1.00 | 29.60 | B |
| ATOM | 970 | CA | ASN | B | 59 | 7.217 | 25.507 | 54.072 | 1.00 | 30.67 | B |
| ATOM | 971 | CB | ASN | B | 59 | 6.558 | 25.095 | 55.400 | 1.00 | 31.78 | B |
| ATOM | 972 | CG | ASN | B | 59 | 7.436 | 25.374 | 56.616 | 1.00 | 34.02 | B |
| ATOM | 973 | OD1 | ASN | B | 59 | 7.427 | 24.603 | 57.590 | 1.00 | 36.16 | B |
| ATOM | 974 | ND2 | ASN | B | 59 | 8.163 | 26.490 | 56.588 | 1.00 | 32.45 | B |
| ATOM | 975 | C | ASN | B | 59 | 8.388 | 24.543 | 53.772 | 1.00 | 30.66 | B |
| ATOM | 976 | O | ASN | B | 59 | 9.262 | 24.370 | 54.609 | 1.00 | 28.83 | B |
| ATOM | 977 | N | GLU | B | 60 | 8.405 | 23.912 | 52.596 | 1.00 | 31.84 | B |
| ATOM | 978 | CA | GLU | B | 60 | 9.484 | 22.959 | 52.263 | 1.00 | 33.09 | B |
| ATOM | 979 | CB | GLU | B | 60 | 8.881 | 21.606 | 51.857 | 1.00 | 33.04 | B |
| ATOM | 980 | CG | GLU | B | 60 | 8.009 | 20.958 | 52.956 | 1.00 | 33.74 | B |
| ATOM | 981 | CD | GLU | B | 60 | 7.326 | 19.647 | 52.512 | 1.00 | 37.38 | B |
| ATOM | 982 | OE1 | GLU | B | 60 | 6.136 | 19.671 | 52.091 | 1.00 | 36.26 | B |
| ATOM | 983 | OE2 | GLU | B | 60 | 7.989 | 18.587 | 52.584 | 1.00 | 37.34 | B |
| ATOM | 984 | C | GLU | B | 60 | 10.407 | 23.450 | 51.155 | 1.00 | 34.34 | B |
| ATOM | 985 | O | GLU | B | 60 | 10.012 | 24.249 | 50.301 | 1.00 | 34.69 | B |
| ATOM | 986 | N | LYS | B | 61 | 11.657 | 22.999 | 51.185 | 1.00 | 34.58 | B |
| ATOM | 987 | CA | LYS | B | 61 | 12.618 | 23.378 | 50.155 | 1.00 | 34.67 | B |
| ATOM | 988 | CB | LYS | B | 61 | 14.023 | 23.474 | 50.757 | 1.00 | 37.32 | B |
| ATOM | 989 | CG | LYS | B | 61 | 15.136 | 23.586 | 49.719 | 1.00 | 40.98 | B |
| ATOM | 990 | CD | LYS | B | 61 | 16.441 | 24.122 | 50.309 | 1.00 | 44.39 | B |
| ATOM | 991 | CE | LYS | B | 61 | 16.911 | 23.344 | 51.533 | 1.00 | 46.53 | B |
| ATOM | 992 | NZ | LYS | B | 61 | 18.090 | 24.028 | 52.184 | 1.00 | 48.46 | B |
| ATOM | 993 | C | LYS | B | 61 | 12.591 | 22.333 | 49.025 | 1.00 | 33.73 | B |
| ATOM | 994 | O | LYS | B | 61 | 12.375 | 21.139 | 49.271 | 1.00 | 32.48 | B |
| ATOM | 995 | N | LEU | B | 62 | 12.784 | 22.793 | 47.791 | 1.00 | 31.87 | B |
| ATOM | 996 | CA | LEU | B | 62 | 12.779 | 21.917 | 46.622 | 1.00 | 32.06 | B |
| ATOM | 997 | CB | LEU | B | 62 | 12.216 | 22.645 | 45.390 | 1.00 | 30.85 | B |
| ATOM | 998 | CG | LEU | B | 62 | 10.758 | 23.104 | 45.390 | 1.00 | 32.13 | B |
| ATOM | 999 | CD1 | LEU | B | 62 | 10.506 | 23.974 | 44.178 | 1.00 | 31.87 | B |
| ATOM | 1000 | CD2 | LEU | B | 62 | 9.845 | 21.878 | 45.365 | 1.00 | 31.68 | B |
| ATOM | 1001 | C | LEU | B | 62 | 14.193 | 21.471 | 46.274 | 1.00 | 32.22 | B |
| ATOM | 1002 | O | LEU | B | 62 | 15.139 | 22.246 | 46.421 | 1.00 | 31.77 | B |
| ATOM | 1003 | N | PRO | B | 63 | 14.349 | 20.212 | 45.813 | 1.00 | 32.35 | B |
| ATOM | 1004 | CD | PRO | B | 63 | 13.289 | 19.184 | 45.816 | 1.00 | 33.22 | B |
| ATOM | 1005 | CA | PRO | B | 63 | 15.639 | 19.637 | 45.416 | 1.00 | 32.16 | B |
| ATOM | 1006 | CB | PRO | B | 63 | 15.319 | 18.163 | 45.156 | 1.00 | 32.18 | B |
| ATOM | 1007 | CG | PRO | B | 63 | 14.067 | 17.918 | 45.977 | 1.00 | 33.74 | B |
| ATOM | 1008 | C | PRO | B | 63 | 16.027 | 20.330 | 44.131 | 1.00 | 32.29 | B |
| ATOM | 1009 | O | PRO | B | 63 | 15.142 | 20.819 | 43.403 | 1.00 | 30.11 | B |
| ATOM | 1010 | N | GLU | B | 64 | 17.333 | 20.353 | 43.832 | 1.00 | 32.20 | B |
| ATOM | 1011 | CA | GLU | B | 64 | 17.840 | 21.002 | 42.619 | 1.00 | 31.44 | B |
| ATOM | 1012 | CB | GLU | B | 64 | 19.372 | 20.977 | 42.567 | 1.00 | 33.66 | B |
| ATOM | 1013 | CG | GLU | B | 64 | 20.040 | 21.984 | 43.496 | 1.00 | 39.13 | B |
| ATOM | 1014 | CD | GLU | B | 64 | 19.571 | 23.414 | 43.252 | 1.00 | 40.51 | B |
| ATOM | 1015 | OE1 | GLU | B | 64 | 19.507 | 23.828 | 42.065 | 1.00 | 41.69 | B |
| ATOM | 1016 | OE2 | GLU | B | 64 | 19.273 | 24.120 | 44.250 | 1.00 | 44.35 | B |
| ATOM | 1017 | C | GLU | B | 64 | 17.331 | 20.433 | 41.313 | 1.00 | 30.81 | B |
| ATOM | 1018 | O | GLU | B | 64 | 17.141 | 21.178 | 40.336 | 1.00 | 30.24 | B |
| ATOM | 1019 | N | TYR | B | 65 | 17.125 | 19.123 | 41.244 | 1.00 | 28.06 | B |
| ATOM | 1020 | CA | TYR | B | 65 | 16.654 | 18.588 | 39.971 | 1.00 | 28.85 | B |
| ATOM | 1021 | CB | TYR | B | 65 | 16.779 | 17.059 | 39.937 | 1.00 | 28.86 | B |
| ATOM | 1022 | CG | TYR | B | 65 | 15.746 | 16.329 | 40.744 | 1.00 | 28.28 | B |
| ATOM | 1023 | CD1 | TYR | B | 65 | 14.620 | 15.788 | 40.124 | 1.00 | 30.60 | B |
| ATOM | 1024 | CE1 | TYR | B | 65 | 13.701 | 15.042 | 40.828 | 1.00 | 29.77 | B |
| ATOM | 1025 | CD2 | TYR | B | 65 | 15.916 | 16.118 | 42.106 | 1.00 | 27.97 | B |
| ATOM | 1026 | CE2 | TYR | B | 65 | 14.989 | 15.375 | 42.837 | 1.00 | 29.71 | B |
| ATOM | 1027 | CZ | TYR | B | 65 | 13.890 | 14.838 | 42.190 | 1.00 | 30.50 | B |
| ATOM | 1028 | OH | TYR | B | 65 | 12.966 | 14.095 | 42.883 | 1.00 | 30.91 | B |
| ATOM | 1029 | C | TYR | B | 65 | 15.209 | 19.034 | 39.714 | 1.00 | 27.21 | B |
| ATOM | 1030 | O | TYR | B | 65 | 14.775 | 19.109 | 38.572 | 1.00 | 27.93 | B |
| ATOM | 1031 | N | ILE | B | 66 | 14.466 | 19.327 | 40.775 | 1.00 | 27.73 | B |
| ATOM | 1032 | CA | ILE | B | 66 | 13.088 | 19.813 | 40.616 | 1.00 | 29.70 | B |
| ATOM | 1033 | CB | ILE | B | 66 | 12.312 | 19.780 | 41.951 | 1.00 | 30.45 | B |
| ATOM | 1034 | CG2 | ILE | B | 66 | 10.896 | 20.377 | 41.761 | 1.00 | 31.18 | B |
| ATOM | 1035 | CG1 | ILE | B | 66 | 12.262 | 18.347 | 42.500 | 1.00 | 33.52 | B |
| ATOM | 1036 | CD1 | ILE | B | 66 | 11.287 | 17.460 | 41.858 | 1.00 | 30.53 | B |
| ATOM | 1037 | C | ILE | B | 66 | 13.147 | 21.280 | 40.138 | 1.00 | 30.08 | B |
| ATOM | 1038 | O | ILE | B | 66 | 12.441 | 21.665 | 39.215 | 1.00 | 30.26 | B |
| ATOM | 1039 | N | LYS | B | 67 | 14.001 | 22.084 | 40.766 | 1.00 | 30.53 | B |
| ATOM | 1040 | CA | LYS | B | 67 | 14.145 | 23.500 | 40.411 | 1.00 | 33.59 | B |
| ATOM | 1041 | CB | LYS | B | 67 | 15.194 | 24.185 | 41.290 | 1.00 | 33.18 | B |
| ATOM | 1042 | CG | LYS | B | 67 | 15.064 | 23.881 | 42.786 | 1.00 | 37.68 | B |
| ATOM | 1043 | CD | LYS | B | 67 | 14.811 | 25.143 | 43.630 | 1.00 | 40.72 | B |
| ATOM | 1044 | CE | LYS | B | 67 | 15.987 | 26.095 | 43.620 | 1.00 | 41.58 | B |
| ATOM | 1045 | NZ | LYS | B | 67 | 17.116 | 25.648 | 44.477 | 1.00 | 45.16 | B |
| ATOM | 1046 | C | LYS | B | 67 | 14.570 | 23.620 | 38.955 | 1.00 | 34.20 | B |
| ATOM | 1047 | O | LYS | B | 67 | 14.023 | 24.435 | 38.201 | 1.00 | 34.67 | B |
| ATOM | 1048 | N | GLN | B | 68 | 15.554 | 22.812 | 38.569 | 1.00 | 33.52 | B |
| ATOM | 1049 | CA | GLN | B | 68 | 16.047 | 22.801 | 37.208 | 1.00 | 34.63 | B |
| ATOM | 1050 | CB | GLN | B | 68 | 16.992 | 21.618 | 37.010 | 1.00 | 39.08 | B |
| ATOM | 1051 | CG | GLN | B | 68 | 18.215 | 21.649 | 37.878 | 1.00 | 44.05 | B |
| ATOM | 1052 | CD | GLN | B | 68 | 19.392 | 22.243 | 37.150 | 1.00 | 47.61 | B |
| ATOM | 1053 | OE1 | GLN | B | 68 | 19.400 | 23.442 | 36.815 | 1.00 | 49.22 | B |
| ATOM | 1054 | NE2 | GLN | B | 68 | 20.401 | 21.405 | 36.880 | 1.00 | 48.50 | B |
| ATOM | 1055 | C | GLN | B | 68 | 14.859 | 22.637 | 36.270 | 1.00 | 33.72 | B |
| ATOM | 1056 | O | GLN | B | 68 | 14.764 | 23.295 | 35.234 | 1.00 | 34.87 | B |
| ATOM | 1057 | N | LYS | B | 69 | 13.947 | 21.741 | 36.614 | 1.00 | 31.19 | B |
| ATOM | 1058 | CA | LYS | B | 69 | 12.803 | 21.531 | 35.741 | 1.00 | 30.30 | B |
| ATOM | 1059 | CB | LYS | B | 69 | 12.076 | 20.237 | 36.126 | 1.00 | 27.57 | B |
| ATOM | 1060 | CG | LYS | B | 69 | 12.629 | 19.057 | 35.327 | 1.00 | 29.07 | B |
| ATOM | 1061 | CD | LYS | B | 69 | 12.272 | 17.668 | 35.859 | 1.00 | 24.54 | B |
| ATOM | 1062 | CE | LYS | B | 69 | 12.698 | 16.604 | 34.821 | 1.00 | 25.27 | B |
| ATOM | 1063 | NZ | LYS | B | 69 | 12.601 | 15.215 | 35.374 | 1.00 | 20.26 | B |
| ATOM | 1064 | C | LYS | B | 69 | 11.882 | 22.748 | 35.763 | 1.00 | 29.46 | B |
| ATOM | 1065 | O | LYS | B | 69 | 11.308 | 23.111 | 34.734 | 1.00 | 30.15 | B |
| ATOM | 1066 | N | LEU | B | 70 | 11.765 | 23.378 | 36.928 | 1.00 | 29.80 | B |
| ATOM | 1067 | CA | LEU | B | 70 | 10.957 | 24.588 | 37.081 | 1.00 | 33.21 | B |
| ATOM | 1068 | CB | LEU | B | 70 | 10.965 | 25.076 | 38.527 | 1.00 | 31.58 | B |
| ATOM | 1069 | CG | LEU | B | 70 | 9.957 | 24.413 | 39.450 | 1.00 | 34.15 | B |
| ATOM | 1070 | CD1 | LEU | B | 70 | 10.146 | 24.954 | 40.866 | 1.00 | 33.82 | B |
| ATOM | 1071 | CD2 | LEU | B | 70 | 8.546 | 24.706 | 38.942 | 1.00 | 33.95 | B |
| ATOM | 1072 | C | LEU | B | 70 | 11.470 | 25.713 | 36.192 | 1.00 | 32.31 | B |
| ATOM | 1073 | O | LEU | B | 70 | 10.684 | 26.459 | 35.636 | 1.00 | 33.63 | B |
| ATOM | 1074 | N | GLN | B | 71 | 12.786 | 25.834 | 36.053 | 1.00 | 35.18 | B |
| ATOM | 1075 | CA | GLN | B | 71 | 13.356 | 26.899 | 35.225 | 1.00 | 36.36 | B |
| ATOM | 1076 | CB | GLN | B | 71 | 14.887 | 26.877 | 35.265 | 1.00 | 38.30 | B |
| ATOM | 1077 | CG | GLN | B | 71 | 15.472 | 26.777 | 36.654 | 1.00 | 43.28 | B |
| ATOM | 1078 | CD | GLN | B | 71 | 17.001 | 26.759 | 36.647 | 1.00 | 47.08 | B |
| ATOM | 1079 | OE1 | GLN | B | 71 | 17.633 | 26.351 | 37.626 | 1.00 | 49.47 | B |
| ATOM | 1080 | NE2 | GLN | B | 71 | 17.599 | 27.207 | 35.541 | 1.00 | 49.59 | B |
| ATOM | 1081 | C | GLN | B | 71 | 12.908 | 26.817 | 33.769 | 1.00 | 36.22 | B |
| ATOM | 1082 | O | GLN | B | 71 | 12.818 | 27.858 | 33.099 | 1.00 | 35.03 | B |
| ATOM | 1083 | N | LEU | B | 72 | 12.622 | 25.606 | 33.273 | 1.00 | 34.21 | B |
| ATOM | 1084 | CA | LEU | B | 72 | 12.201 | 25.448 | 31.874 | 1.00 | 33.60 | B |
| ATOM | 1085 | CB | LEU | B | 72 | 12.164 | 23.966 | 31.455 | 1.00 | 34.75 | B |
| ATOM | 1086 | CG | LEU | B | 72 | 13.423 | 23.089 | 31.537 | 1.00 | 34.77 | B |
| ATOM | 1087 | CD1 | LEU | B | 72 | 13.039 | 21.606 | 31.382 | 1.00 | 34.45 | B |
| ATOM | 1088 | CD2 | LEU | B | 72 | 14.401 | 23.511 | 30.442 | 1.00 | 34.92 | B |
| ATOM | 1089 | C | LEU | B | 72 | 10.817 | 26.043 | 31.643 | 1.00 | 33.64 | B |
| ATOM | 1090 | O | LEU | B | 72 | 10.400 | 26.194 | 30.494 | 1.00 | 31.65 | B |
| ATOM | 1091 | N | LEU | B | 73 | 10.116 | 26.392 | 32.724 | 1.00 | 32.55 | B |
| ATOM | 1092 | CA | LEU | B | 73 | 8.764 | 26.947 | 32.608 | 1.00 | 33.85 | B |
| ATOM | 1093 | CB | LEU | B | 73 | 7.883 | 26.432 | 33.754 | 1.00 | 35.05 | B |
| ATOM | 1094 | CG | LEU | B | 73 | 7.891 | 24.912 | 33.938 | 1.00 | 37.23 | B |
| ATOM | 1095 | CD1 | LEU | B | 73 | 7.380 | 24.551 | 35.344 | 1.00 | 38.68 | B |
| ATOM | 1096 | CD2 | LEU | B | 73 | 7.056 | 24.260 | 32.843 | 1.00 | 37.27 | B |
| ATOM | 1097 | C | LEU | B | 73 | 8.717 | 28.476 | 32.594 | 1.00 | 33.24 | B |
| ATOM | 1098 | O | LEU | B | 73 | 7.746 | 29.058 | 32.128 | 1.00 | 34.63 | B |
| ATOM | 1099 | N | SER | B | 74 | 9.764 | 29.112 | 33.111 | 1.00 | 32.48 | B |
| ATOM | 1100 | CA | SER | B | 74 | 9.857 | 30.563 | 33.164 | 1.00 | 29.94 | B |
| ATOM | 1101 | CB | SER | B | 74 | 11.218 | 30.969 | 33.719 | 1.00 | 30.18 | B |
| ATOM | 1102 | OG | SER | B | 74 | 11.398 | 30.395 | 35.006 | 1.00 | 35.73 | B |
| ATOM | 1103 | C | SER | B | 74 | 9.650 | 31.210 | 31.800 | 1.00 | 28.75 | B |
| ATOM | 1104 | O | SER | B | 74 | 10.313 | 30.853 | 30.816 | 1.00 | 28.50 | B |
| ATOM | 1105 | N | SER | B | 75 | 8.720 | 32.158 | 31.727 | 1.00 | 26.58 | B |
| ATOM | 1106 | CA | SER | B | 75 | 8.469 | 32.850 | 30.463 | 1.00 | 23.76 | B |
| ATOM | 1107 | CB | SER | B | 75 | 7.521 | 32.043 | 29.583 | 1.00 | 25.25 | B |
| ATOM | 1108 | OG | SER | B | 75 | 6.220 | 31.915 | 30.161 | 1.00 | 25.57 | B |
| ATOM | 1109 | C | SER | B | 75 | 7.901 | 34.240 | 30.670 | 1.00 | 23.10 | B |
| ATOM | 1110 | O | SER | B | 75 | 7.564 | 34.634 | 31.779 | 1.00 | 19.92 | B |
| ATOM | 1111 | N | ILE | B | 76 | 7.875 | 34.982 | 29.577 | 1.00 | 22.80 | B |
| ATOM | 1112 | CA | ILE | B | 76 | 7.362 | 36.330 | 29.504 | 1.00 | 23.91 | B |
| ATOM | 1113 | CB | ILE | B | 76 | 8.500 | 37.358 | 29.251 | 1.00 | 26.24 | B |
| ATOM | 1114 | CG2 | ILE | B | 76 | 7.908 | 38.709 | 28.915 | 1.00 | 27.13 | B |
| ATOM | 1115 | CG1 | ILE | B | 76 | 9.382 | 37.483 | 30.498 | 1.00 | 28.59 | B |
| ATOM | 1116 | CD1 | ILE | B | 76 | 10.458 | 38.553 | 30.372 | 1.00 | 33.23 | B |
| ATOM | 1117 | C | ILE | B | 76 | 6.479 | 36.228 | 28.252 | 1.00 | 23.80 | B |
| ATOM | 1118 | O | ILE | B | 76 | 6.966 | 35.838 | 27.166 | 1.00 | 22.78 | B |
| ATOM | 1119 | N | LEU | B | 77 | 5.194 | 36.518 | 28.407 | 1.00 | 23.20 | B |
| ATOM | 1120 | CA | LEU | B | 77 | 4.239 | 36.447 | 27.286 | 1.00 | 23.99 | B |
| ATOM | 1121 | CB | LEU | B | 77 | 3.244 | 35.304 | 27.502 | 1.00 | 23.91 | B |
| ATOM | 1122 | CG | LEU | B | 77 | 2.153 | 35.143 | 26.409 | 1.00 | 26.66 | B |
| ATOM | 1123 | CD1 | LEU | B | 77 | 1.935 | 33.666 | 26.098 | 1.00 | 27.56 | B |
| ATOM | 1124 | CD2 | LEU | B | 77 | 0.849 | 35.772 | 26.875 | 1.00 | 26.92 | B |
| ATOM | 1125 | C | LEU | B | 77 | 3.468 | 37.755 | 27.177 | 1.00 | 24.42 | B |
| ATOM | 1126 | O | LEU | B | 77 | 3.112 | 38.356 | 28.196 | 1.00 | 22.52 | B |
| ATOM | 1127 | N | LEU | B | 78 | 3.269 | 38.218 | 25.945 | 1.00 | 23.94 | B |
| ATOM | 1128 | CA | LEU | B | 78 | 2.496 | 39.425 | 25.680 | 1.00 | 25.25 | B |
| ATOM | 1129 | CB | LEU | B | 78 | 3.403 | 40.559 | 25.195 | 1.00 | 26.26 | B |
| ATOM | 1130 | CG | LEU | B | 78 | 4.448 | 41.009 | 26.230 | 1.00 | 27.97 | B |
| ATOM | 1131 | CD1 | LEU | B | 78 | 5.575 | 41.856 | 25.603 | 1.00 | 29.40 | B |
| ATOM | 1132 | CD2 | LEU | B | 78 | 3.700 | 41.789 | 27.297 | 1.00 | 31.48 | B |
| ATOM | 1133 | C | LEU | B | 78 | 1.541 | 39.044 | 24.567 | 1.00 | 26.11 | B |
| ATOM | 1134 | O | LEU | B | 78 | 1.973 | 38.505 | 23.551 | 1.00 | 24.70 | B |
| ATOM | 1135 | N | MET | B | 79 | 0.249 | 39.281 | 24.772 | 1.00 | 25.67 | B |
| ATOM | 1136 | CA | MET | B | 79 | −0.765 | 38.995 | 23.754 | 1.00 | 27.63 | B |
| ATOM | 1137 | CB | MET | B | 79 | −1.578 | 37.753 | 24.106 | 1.00 | 28.06 | B |
| ATOM | 1138 | CG | MET | B | 79 | −2.443 | 37.323 | 22.926 | 1.00 | 35.25 | B |
| ATOM | 1139 | SD | MET | B | 79 | −3.262 | 35.745 | 23.168 | 1.00 | 40.80 | B |
| ATOM | 1140 | CE | MET | B | 79 | −2.237 | 34.993 | 24.422 | 1.00 | 37.94 | B |
| ATOM | 1141 | C | MET | B | 79 | −1.715 | 40.203 | 23.604 | 1.00 | 27.55 | B |
| ATOM | 1142 | O | MET | B | 79 | −2.243 | 40.710 | 24.602 | 1.00 | 24.75 | B |
| ATOM | 1143 | N | PHE | B | 80 | −1.906 | 40.670 | 22.370 | 1.00 | 27.13 | B |
| ATOM | 1144 | CA | PHE | B | 80 | −2.770 | 41.822 | 22.098 | 1.00 | 30.08 | B |
| ATOM | 1145 | CB | PHE | B | 80 | −1.965 | 43.041 | 21.609 | 1.00 | 29.04 | B |
| ATOM | 1146 | CG | PHE | B | 80 | −0.704 | 43.324 | 22.396 | 1.00 | 30.01 | B |
| ATOM | 1147 | CD1 | PHE | B | 80 | 0.462 | 42.587 | 22.164 | 1.00 | 30.40 | B |
| ATOM | 1148 | CD2 | PHE | B | 80 | −0.672 | 44.359 | 23.337 | 1.00 | 31.06 | B |
| ATOM | 1149 | CE1 | PHE | B | 80 | 1.647 | 42.875 | 22.857 | 1.00 | 31.13 | B |
| ATOM | 1150 | CE2 | PHE | B | 80 | 0.511 | 44.665 | 24.045 | 1.00 | 31.57 | B |
| ATOM | 1151 | CZ | PHE | B | 80 | 1.675 | 43.916 | 23.800 | 1.00 | 30.17 | B |
| ATOM | 1152 | C | PHE | B | 80 | −3.770 | 41.508 | 20.996 | 1.00 | 33.16 | B |
| ATOM | 1153 | O | PHE | B | 80 | −3.456 | 40.766 | 20.050 | 1.00 | 30.01 | B |
| ATOM | 1154 | N | SER | B | 81 | −4.978 | 42.061 | 21.112 | 1.00 | 35.97 | B |
| ATOM | 1155 | CA | SER | B | 81 | −5.976 | 41.897 | 20.048 | 1.00 | 40.51 | B |
| ATOM | 1156 | CB | SER | B | 81 | −7.337 | 42.442 | 20.494 | 1.00 | 39.87 | B |
| ATOM | 1157 | OG | SER | B | 81 | −7.832 | 41.698 | 21.591 | 1.00 | 40.39 | B |
| ATOM | 1158 | C | SER | B | 81 | −5.438 | 42.744 | 18.868 | 1.00 | 42.34 | B |
| ATOM | 1159 | O | SER | B | 81 | −5.008 | 43.874 | 19.063 | 1.00 | 44.86 | B |
| ATOM | 1160 | N | ASN | B | 82 | −5.463 | 42.206 | 17.654 | 1.00 | 44.80 | B |
| ATOM | 1161 | CA | ASN | B | 82 | −4.951 | 42.914 | 16.477 | 1.00 | 46.28 | B |
| ATOM | 1162 | CB | ASN | B | 82 | −4.259 | 41.906 | 15.544 | 1.00 | 46.09 | B |
| ATOM | 1163 | CG | ASN | B | 82 | −3.309 | 42.565 | 14.537 | 1.00 | 46.70 | B |
| ATOM | 1164 | OD1 | ASN | B | 82 | −2.677 | 41.874 | 13.716 | 1.00 | 46.56 | B |
| ATOM | 1165 | ND2 | ASN | B | 82 | −3.203 | 43.891 | 14.593 | 1.00 | 45.41 | B |
| ATOM | 1166 | C | ASN | B | 82 | −6.073 | 43.653 | 15.722 | 1.00 | 48.50 | B |
| ATOM | 1167 | O | ASN | B | 82 | −6.410 | 43.239 | 14.578 | 1.00 | 47.94 | B |
| ATOM | 1168 | OXT | ASN | B | 82 | −6.611 | 44.640 | 16.292 | 1.00 | 51.85 | B |
| ATOM | 1169 | OH2 | TIP | S | 1 | 1.508 | 24.728 | 50.569 | 1.00 | 21.12 | S |
| ATOM | 1170 | OH2 | TIP | S | 2 | −4.532 | 41.128 | 52.790 | 1.00 | 22.93 | S |
| ATOM | 1171 | OH2 | TIP | S | 3 | 0.453 | 33.543 | 46.169 | 1.00 | 21.41 | S |
| ATOM | 1172 | OH2 | TIP | S | 4 | 8.870 | 11.544 | 43.348 | 1.00 | 25.23 | S |
| ATOM | 1173 | OH2 | TIP | S | 5 | −3.457 | 47.896 | 37.725 | 1.00 | 21.86 | S |
| ATOM | 1174 | OH2 | TIP | S | 6 | 11.989 | 7.249 | 38.203 | 1.00 | 25.35 | S |
| ATOM | 1175 | OH2 | TIP | S | 7 | 1.880 | 40.091 | 46.556 | 1.00 | 29.15 | S |
| ATOM | 1176 | OH2 | TIP | S | 8 | 2.444 | 35.387 | 45.395 | 1.00 | 29.58 | S |
| ATOM | 1177 | OH2 | TIP | S | 9 | −10.635 | 60.279 | 36.514 | 1.00 | 25.71 | S |
| ATOM | 1178 | OH2 | TIP | S | 10 | −5.178 | 50.690 | 47.482 | 1.00 | 27.75 | S |
| ATOM | 1179 | OH2 | TIP | S | 11 | 5.346 | 13.571 | 49.415 | 1.00 | 29.60 | S |
| ATOM | 1180 | OH2 | TIP | S | 12 | 11.036 | 7.211 | 41.061 | 1.00 | 25.43 | S |
| ATOM | 1181 | OH2 | TIP | S | 13 | 2.572 | 14.979 | 39.851 | 1.00 | 25.44 | S |
| ATOM | 1182 | OH2 | TIP | S | 14 | −0.581 | 43.317 | 42.332 | 1.00 | 33.47 | S |
| ATOM | 1183 | OH2 | TIP | S | 15 | −12.815 | 55.716 | 40.968 | 1.00 | 30.28 | S |
| ATOM | 1184 | OH2 | TIP | S | 16 | −0.965 | 48.449 | 38.790 | 1.00 | 25.80 | S |
| ATOM | 1185 | OH2 | TIP | S | 17 | −17.201 | 44.033 | 35.905 | 1.00 | 29.81 | S |
| ATOM | 1186 | OH2 | TIP | S | 18 | −2.352 | 31.966 | 50.012 | 1.00 | 21.46 | S |
| ATOM | 1187 | OH2 | TIP | S | 19 | −12.888 | 38.321 | 27.123 | 1.00 | 31.06 | S |
| ATOM | 1188 | OH2 | TIP | S | 20 | 0.353 | 20.226 | 43.760 | 1.00 | 34.64 | S |
| ATOM | 1189 | OH2 | TIP | S | 21 | 10.886 | 7.638 | 30.517 | 1.00 | 32.87 | S |
| ATOM | 1190 | OH2 | TIP | S | 22 | −6.652 | 39.779 | 46.435 | 1.00 | 31.11 | S |
| ATOM | 1191 | OH2 | TIP | S | 23 | −9.631 | 51.555 | 41.700 | 1.00 | 29.23 | S |
| ATOM | 1192 | OH2 | TIP | S | 24 | −6.268 | 37.267 | 45.848 | 1.00 | 30.09 | S |
| ATOM | 1193 | OH2 | TIP | S | 25 | −10.305 | 30.700 | 43.071 | 1.00 | 34.11 | S |
| ATOM | 1194 | OH2 | TIP | S | 26 | −13.571 | 38.030 | 48.036 | 1.00 | 38.04 | S |
| ATOM | 1195 | OH2 | TIP | S | 27 | 0.283 | 14.726 | 41.020 | 1.00 | 33.01 | S |
| ATOM | 1196 | OH2 | TIP | S | 28 | 16.076 | 17.853 | 36.341 | 1.00 | 32.65 | S |
| ATOM | 1197 | OH2 | TIP | S | 29 | 0.078 | 30.990 | 51.479 | 1.00 | 35.59 | S |
| ATOM | 1198 | OH2 | TIP | S | 30 | −16.819 | 48.859 | 31.799 | 1.00 | 33.87 | S |
| ATOM | 1199 | OH2 | TIP | S | 31 | 11.178 | 22.910 | 27.196 | 1.00 | 34.94 | S |
| ATOM | 1200 | OH2 | TIP | S | 32 | 4.359 | 17.883 | 51.741 | 1.00 | 34.82 | S |
| ATOM | 1201 | OH2 | TIP | S | 33 | 2.022 | 32.446 | 50.376 | 1.00 | 23.48 | S |
| ATOM | 1202 | OH2 | TIP | S | 34 | 19.034 | 19.266 | 46.006 | 1.00 | 35.73 | S |
| ATOM | 1203 | OH2 | TIP | S | 35 | 10.267 | 32.663 | 42.312 | 1.00 | 42.74 | S |
| ATOM | 1204 | OH2 | TIP | S | 36 | 8.286 | 1.858 | 35.678 | 1.00 | 29.10 | S |
| ATOM | 1205 | OH2 | TIP | S | 37 | −5.005 | 55.786 | 42.115 | 1.00 | 39.52 | S |
| ATOM | 1206 | OH2 | TIP | S | 38 | −7.453 | 59.109 | 38.085 | 1.00 | 40.32 | S |
| ATOM | 1207 | OH2 | TIP | S | 39 | −1.225 | 48.872 | 43.438 | 1.00 | 36.53 | S |
| ATOM | 1208 | OH2 | TIP | S | 40 | 5.207 | 13.791 | 15.362 | 1.00 | 41.72 | S |
| ATOM | 1209 | OH2 | TIP | S | 41 | 5.160 | 10.320 | 35.567 | 1.00 | 31.09 | S |
| ATOM | 1210 | OH2 | TIP | S | 42 | 18.752 | 17.356 | 43.075 | 1.00 | 37.74 | S |
| ATOM | 1211 | OH2 | TIP | S | 43 | −18.397 | 40.367 | 29.850 | 1.00 | 48.05 | S |
| ATOM | 1212 | OH2 | TIP | S | 44 | 8.184 | 37.232 | 40.970 | 1.00 | 40.60 | S |
| ATOM | 1213 | OH2 | TIP | S | 45 | −6.617 | 31.751 | 32.951 | 1.00 | 40.27 | S |
| ATOM | 1214 | OH2 | TIP | S | 46 | −8.475 | 47.711 | 49.511 | 1.00 | 38.51 | S |
| ATOM | 1215 | OH2 | TIP | S | 47 | −7.813 | 36.040 | 47.740 | 1.00 | 41.75 | S |
| ATOM | 1216 | OH2 | TIP | S | 48 | 6.688 | 38.985 | 40.094 | 1.00 | 37.02 | S |
| ATOM | 1217 | OH2 | TIP | S | 49 | −12.153 | 36.097 | 28.343 | 1.00 | 43.38 | S |
| ATOM | 1218 | OH2 | TIP | S | 50 | −19.218 | 44.577 | 45.754 | 1.00 | 39.99 | S |
| ATOM | 1219 | OH2 | TIP | S | 51 | 3.811 | 7.715 | 44.758 | 1.00 | 38.01 | S |
| ATOM | 1220 | OH2 | TIP | S | 52 | −5.378 | 33.843 | 35.965 | 1.00 | 54.23 | S |
| ATOM | 1221 | OH2 | TIP | S | 53 | −4.266 | 33.146 | 37.939 | 1.00 | 42.29 | S |
| ATOM | 1222 | OH2 | TIP | S | 54 | −2.398 | 31.670 | 38.304 | 1.00 | 47.47 | S |
| ATOM | 1223 | OH2 | TIP | S | 55 | 2.394 | 31.399 | 38.501 | 1.00 | 49.33 | S |
| ATOM | 1224 | OH2 | TIP | S | 56 | 4.080 | 30.038 | 37.667 | 1.00 | 37.16 | S |
| ATOM | 1225 | OH2 | TIP | S | 57 | 4.352 | 28.531 | 35.734 | 1.00 | 51.95 | S |
| ATOM | 1226 | OH2 | TIP | S | 58 | 3.223 | 29.525 | 33.569 | 1.00 | 42.32 | S |
| END | | | | | | | | | | | |

Full Citations for References Referred to in the Specification
1. Donaldson, M. M., Tavares, A. A., Hagan, I. M., Nigg, E. A. & Glover, D. M. J. Cell Sci. 114, 2357-2358. (2001).
2. Glover, D. M. , Hagan, I. M. & Tavares, A. A. Genes Dev. 12, 3777-3787. (1998).
3. Nigg, E. A. Curr. Opin. Cell Biol. 10, 776-783. (1998).
4. Sunkel, C. E. & Glover, D. M. J. Cell. Sci. 89, 25-38. (1988).
5. Carmena, M. et al. J. Cell Biol. 143, 659-671. (1998).
6. Ohkura, H., Hagan, I. M. & Glover, D. M. Genes Dev. 9, 1059-1073. (1995).
7. Simchen, G., Kassir, Y., Horesh-Cabilly, O. & Friedmann, A. Mol. Gen. Genet 184, 46-51 (1981).
8. Song, S. & Lee, K. S. J. Cell. Biol. 152, 451-469. (200 1).
9. Hudson, J. W. et al. Curr. Biol. 11, 441-446. (2001).
10. Lee, K. S. & Erikson, R. L. Mol. Cell. Biol. 17, 3408-3417. (1997).
11. Ouyang, B. et al. J. Biol. Chem. 272, 28646-28651. (1997).
12. Golsteyn, R. M., Mundt, K. E., Fry, A. M. & Nigg, E. A. J. Cell Biol. 129, 1617-1628. (1995).
13. Lee, K. S., Grenfell, T. Z., Yarm, F. R. & Erikson, R. L. Proc. Natl. Acad. Sci. USA 95, 9301-9306. (1998).
14. Wang, Q. et al. Mol. Cell. Biol. 22, 3450-3459. (2002).
15. Song, S., Grenfell, T. Z., Garfield, S., Erikson, R. L. & Lee, K. S. Mol. Cell. Biol. 20, 286-298. (2000).
16. Mulvihill, D. P., Petersen, J., Ohkura, H., Glover, D. M. & Hagan, I. M. Mol. Biol. Cell 10, 2771-2785. (1999).
17. Adams, R. R., Tavares, A. A., Salzberg, A., Bellen, H. J. & Glover, D. M. Genes. Dev. 12, 1483-1494. (1998).
18. Madej, T., Gibrat, J. F. & Bryant, S. H. Proteins 23, 356-369. (1995).
19. Wheeler, D. L. et al. Nucleic Acids Res. 30, 13-16. (2002).
20. Schultz, J., Milpetz, F., Bork, P. & Ponting, C. P. Proc. Natl. Acad. Sci. USA 95, 5857-5864. (1998).
21. Falquet, L. et al. Nucleic Acids Res. 30, 235-238. (2002).
22. Jang, Y. J., Lin, C. Y., Ma, S. & Erikson, R. L. Proc. Natl. Acad. Sci. U S A 99, 1984-1989. (2002).
23. Ho, Y. et al. Nature 415, 180-183. (2002).
24. Feng, Y. et al. Biochem. J. 339, 435-442. (1999).
25. Hu, F. et al. Cell 107, 655-665. (2001).
26. Bahler, J. et al. J. Cell Biol. 143, 1603-1616. (1998).
27. Smits, V. A. et al. Nat. Cell Biol. 2, 672-676. (2000).
28. Simizu, S. & Osada, H. Nat. Cell Biol. 2, 852-854. (2000).
29. Wolf, G. et al. Oncogene 14, 543-549. (1997).
30. Dietzmann, K., Kirches, E., von, B., Jachau, K. & Mawrin, C. J. Neurooncol. 53, 1-11. (2001).
31. Tokumitsu, Y. et al. Int. J. Oncol. 15, 687-692. (1999).
32. Knecht, R. et al. Cancer Res. 59, 2794-2797. (1999).
33. Smith, M. R. et al. Biochem. Biophys. Res. Commun. 234, 397-405. (1997).
34. Cogswell, J. P., Brown, C. E., Bisi, J. E & Neill, S. D. Cell Growth Differ. 11, 615-623. (2000).
35. Luo, Y. et al. Nature Struct. Biol. 8, 1031-1036. (2001).
36. Otwinowski, Z. & Minor, W. Methods Enzymol. 276, 307-326. (1997).
37. Brunger, A. T. et al. Acta. Cryst. D54, 905-921. (1998).
38. de La Fortelle, E. & Bricogne, G. Methods Enzymol. 276, 472-494. (1997).
39. Jones, T. A., Zou, J. Y., Cowan, S. W. & Kjeldgaard, M. Acta. Cryst. A47, 110-119. (1991).
40. Laskowski, R. A., MacArthur, M. W, Moss, D. S. & Thornton, J. M. J. Appl. Crystallgr. 26, 283-291 (1993).
41. Carson, M. J. Appl. Crystallgr. 24, 958-961. (1991).
42. Nicholls, A., Sharp, K. A. & Honig, B. Proteins Struct. Funct. Genet. II, 281-296. (1991).

1. An isolated binding pocket of a polo domain.

2. An isolated binding pocket of claim 1 wherein the polo domain is a polo domain of Sak or Plk1.

3. A crystal comprising a binding pocket of a polo domain.

4. A crystal as claimed in claim 3 wherein the polo domain is a polo domain of Sak or Plk1.

5. Molecules or molecular complexes that comprise all or parts of a binding pocket as claimed in claim 1, or a homolog of the binding pocket that has similar structure and shape.

6. A crystal comprising a binding pocket of claim 1 complexed or associated with a ligand.

7. A crystal as claimed in claim 6 wherein the ligand is a substrate, a cofactor, heavy metal atom, a modulator of the activity of a polo family kinase, or another polo domain.

8. A crystal comprising a binding pocket of a polo domain as claimed in claim 3 and a substrate or analogue thereof, from which it is possible to derive structural data for the substrate.

9. A crystal according to claim 3 wherein the polo domain is derivable from a human cell.

10. A crystal according to claim 3 wherein the crystal comprises a polo domain having a mutation in the part of the enzyme which is involved in phosphorylation.

11. A crystal according to claim 3 having the structural coordinates shown in Table 2.

12. A model of a binding pocket of a polo domain made using a crystal according to claim 3.

13. A computer-readable medium having stored thereon a crystal according to claim 3.

14. A method of determining the secondary and/or tertiary structures of a polypeptide comprising the step of using a crystal according to claim 3.

15. A method of identifying a potential modulator of a polo family kinase comprising the step of applying the structural coordinates of a polo domain or binding pocket thereof of Table 2, to computationally evaluate a test compound for its ability to associate with the polo domain or binding pocket thereof, wherein a test compound that is found to associate with the polo domain or binding pocket thereof is a potential modulator.

16. A method of claim 15 which comprises one or more of the following additional steps:
(a) testing whether the potential modulator is a modulator of the activity of polo family kinases in cellular assays and animal model assays;
(b) modifying the modulator;
(c) optionally rerunning steps (a) or (b); and
(d) preparing a pharmaceutical composition comprising the modulator.

17. A method of screening for a ligand capable of associating with a binding pocket of a polo domain and/or inhibiting or enhancing the atomic contacts of the interactions in a binding pocket of a polo domain comprising the use of a crystal according to claim 3.

18. A pharmaceutical composition comprising a ligand identified in accordance with the method of claim 17, and optionally a pharmaceutically acceptable carrier, diluent, excipient or adjuvant or any combination thereof.

19. A method of treating and/or preventing a disease comprising administering a pharmaceutical composition according to claim 18 to a mammalian patient.

20. A method of conducting a drug discovery business comprising:
(a) providing one or more systems for identifying modulators based on a crystal according to claim 3;
(b) conducting therapeutic profiling of modulators identified in step (a), or further analogs thereof, for efficacy and toxicity in animals; and
(c) formulating a pharmaceutical preparation including one or more modulators identified in step (b) as having an acceptable therapeutic profile.
